(19) United States
(12) Patent Application Publication
(10) Pub. No.: US 2004/0176366 A1
(43) Pub. Date: Sep. 9, 2004

(54) Method of preventing or treating atherosclerosis or restenosis

(76) Inventors: Michael W. Wathen, Thousand Oaks, CA (US); Lynne K. Wathen, Thousand Oaks, CA (US)

Correspondence Address: FLYNN, THIEL, BOUTELL & TANIS, P.C., 2026 RAMBLING ROAD, KALAMAZOO, MI, 49008-1699, US

(21) Appl. No.: 10/651,290

(22) Filed: Aug. 28, 2003

Related U.S. Application Data
(60) Provisional application No. 60/407,563, filed on Aug. 30, 2002.
(60) Provisional application No. 60/469,630, filed on May 9, 2003.

(51) Int. Cl. A61K 31/54; A61K 31/537; A61K 31/495; A61K 31/445; A61K 31/426; A61K 31/421; A61K 31/401

(52) U.S. Cl. 514/227.8; 514/237.2; 514/255.01; 514/317; 514/365; 514/374; 514/400; 514/423

(57) ABSTRACT

The present invention provides a method of preventing or treating atherosclerosis or restenosis in mammals, which comprises administering an effective amount of a compound selected from the group consisting of structures of Formulas X and XII-XV:

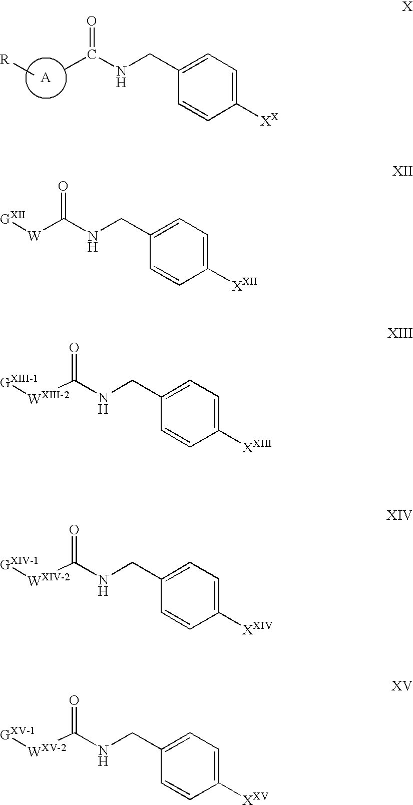

wherein the substituent groups are as defined herein.

### CROSS-REFERENCE TO RELATED APPLICATION

This application claims the benefit of U.S. provisional application Serial No. 60/407,563, filed Aug. 30, 2002, and U.S. provisional application Serial No. 60/469,630, filed May 9, 2003, under 35 USC 119(e)(i), which are incorporated herein by reference.

### BACKGROUND OF THE INVENTION

This invention relates to a method of preventing or treating atherosclerosis and restenosis in mammals.

Atherosclerosis is characterized by the deposition of fatty substances in and fibrosis of the inner layer of the arteries. Restenosis is an accelerated form of athosclerosis that commonly occurs after angioplastic surgery and atherectomy.

Cardiovascular diseases (CVD) contribute substantially to illness and death worldwide and ranks second only to infectious and parasitic diseases as human affliction. Atherosclerosis, a major component of CVD, has properly been considered a public health problem of industrialized countries, accounting for an estimated one third of deaths overall. It has been reported that in the United States alone, atherosclerosis affects one in four persons, causing approximately 42% of all deaths. O'connor et al, “Potential Infectious Etiologies of Atherosclerosis: A Multifactorial Perspective”, Emerging Infectious Disease, Vol. 7, No. 5, September-October 2001.

It has been suggested that the number of chronic infective pathogens which an individual has been exposed independently contribute to the long-term prognosis in patients with documented coronary artery disease. H J Rupprecht et al, “Impact of Viral and Bacterial Infective Burden on Long-term Prognosis in Patients with Coronary Artery Disease. (Circulation (2001) 104:25-31. Seropositivity to multiple herpesviruses is an independent risk factor for death from cardivascular disease and risk is proportional to the number of different herpesviruses that have infected an individual. Other investigators that have suggested a connection between infectious pathogens and atherosclerosis include Espinola-Klein et al, “Impact of Infectious Burden on Extent and Long-Term Prognosis of Atherosclerosis”, Circulation (2002) 105:15-21; O'Connor et al, Supra: and Zhou et al, “Association Between Prior Cytomegalovirus Infection and the Risk of Restenosis after Coronary Atherectomy”, The New England Journal of Medicine (1996). An antiviral drug, Ganciclovir, has been shown to prevent atherosclerosis resulting from CMV infection of rats (K. B. Lemstrom et al. Cytomegalovirus infection-enhanced allograft arteriosclerosis is prevented by DHPG prophylaxis in the rat. Circulation, 1994, 90:1969-1978).

Herpesviruses are believed to be a particular problem in atherosclerosis because they reside latently in an infected individual and can reactivate causing a chronic inflammatory response.

The herpesvirus family contains eight known human viruses; herpes simplex virus type 1 (HSV-1), herpes simplex virus type 2 (HSV-2), varicella zoster virus (VZV), human cytomegalovirus (HCMV), human herpes virus 6 (HHV-6), human herpes virus 7 (HHV-7), Epstein-barr virus (EBV) and human herpes virus 8 (HHV-8). One of the hallmarks of herpesviruses is their ability to establish latent infections in their host and to recur during times of stress or immunosuppression. The human herpesviruses are associated with a diverse set of diseases ranging in severity from mild cold sores to life-threatening illness in immunocompromised patients (Table 1).

TABLE 1
Herpesvirus diseases and treatment

| | Associated Diseases | | |
|---|---|---|---|
| | | Immunocompromised | Marketed |
| Virus | Normal Host | Host | Antivirals |
| HSV-1 | Herpes labialis | Disseminated herpes | Acyclovir |
| | (cold sores) | | Penciclovir |
| HSV-2 | Genital herpes | Disseminated herpes | Acyclovir |
| | | | Valaciclovir |
| | | | Famciclovir |
| VZV | Chicken pox | Herpes zoster | Acyclovir |
| | Herpes zoster | | Valaciclovir |
| | | | Famciclovir |
| CMV | Congenital CMV | Retinitis | Ganciclovir |
| | disease | Pneumonia | Valganciclovir |
| | | GI disease | Foscarnet |
| | | Graft rejection | Cidofovir |
| | | | Formivirsen |
| EBV | Infectious | Lymphomas (PTLD) | None |
| | mononucleosis | | |
| HHV-6 | Exanthem subitum | Graft rejection | None |
| HHV-7 | Exanthem subitum | Graft rejection | None |
| HHV-8 | Kaposi's sarcoma | Kaposi's sarcoma | None |

HSV-1, HCMV, VZV and EBV are ubiquitous viruses with seroprevalence rates in adults of 70-80% for HSV-1 and 90-100% for HCMV, VZV and EBV. Seroprevalence of HSV-2 increases from about 10% in young adults to 35% by age 60. Antibodies to HHV-8 are also found in about 33% of adults in the United States. The high seroprevalence of multiple viruses and their ability to reactivate from latent infections, make these herpesviruses prime candidates for causing chronic inflammatory responses leading to atherosclerosis.

Numerous studies and articles on the epidemiology of the herpesvirus family are in the prior art. Wathen, Michael W., “Non-nucloside inhibitor of herpesviruses”, Rev. Med. Virol, 2002; 12: 167-178; Whitley et al, “Herpes Simplex Viruses”, Clinical Infection Diseases, 1998; 26: 541-55, Cohen, Jeffrey I., “Epstein-Barr Virus Infection”, Medical Progress, Volume 343, Number 7, The New England Journal of Medicine, Aug. 17, 2000, pp. 481-492; Blouvelt et al; “Human Herpes Virus 8 Infection Occurs Following Adolescence in the United States”, The Journal of Infectious Disease, 1997, 176: 771-4; Field, A. Kirk, “Human Cytomegalovirus: challenge opportunities and new drug development”, Antiviral Chemistry and Chemotherapy 10: 219-232.

### Information Disclosure

U.S. Pat. No. 6,239,142 discloses 4-oxo-4,7-dihydrothieno[2,3-b]pyridine-5-carboxamide derivatives, compounds of Formula I and I′ that are useful as antiviral agents. These compounds have now been found to be useful in the method of this invention.

U.S. Pat. No. 6,291,437 describes a method for preventing or retarding the development of atherosclerotic lesions or restenosis comprising administering to a subject, preferably a human, an effective amount of an anti-viral composition directed against CMV, and optionally anti-microbial composition directed against C. pneumoniae.

WO 02/48148 A2 discloses anti-viral compounds and a method of using them for the prophylaxis or treatment of atherosclerosis, coronary artery disease or restenosis.

An antiviral drug, Ganciclovir, has been shown to prevent atherosclerosis resulting from CMV infection of rats (K. B. Lemstrom et al. Cytomegalovirus infection-enhanced allograft arteriosclerosis is prevented by DHPG prophylaxis in the rat. Circulation, 1994, 90:1969-1978).

U.S. Pat. No. 6,239,142 disclosed compounds and their use to treat herpesvirus infections.

WO 02/06513 disclosed method of screening 4-hydroxyquinline, 4-oxo-dihydroquinoline, and 4-oxo-dihydrothienopyridine derivatives as non-nucleoside herpesvirus DNA polymerase inhibitors.

EP 443568 disclosed fused thiophene derivatives, their production and use.

WO 02/04445 disclosed a variety of tricyclic core structures which have antiviral activity against herpesviruses.

WO 02/04444, WO 02/04443, and WO 02/04422 disclosed a variety of bicyclic core structures which have antiviral activity against herpesviruses.

U.S. Pat. No. 6,248,739 disclosed compounds in which the core structure is a quinoline and useful as antivirals against herpesviruses.

### OBJECT OF THE INVENTION

It is the object of this invention to provide a method for preventing or treating atherosclerosis or restenosis in mammals.

It is a further objective of this invention to provide a method for prophylaxis of atherosclerosis and treat patients who have atherosclerosis.

It is still a further objective of the invention to provide a method that prevents or ameliorates the occurrence of restenosis in patients anticipating coronary atheroscopy or angioplasty.

### SUMMARY OF THE INVENTION

A method of preventing or treating atherosclerosis or restenosis in a mammal, comprising administering to said mammal an effective amount of a compound selected from the group consisting of structures of Formula X, Formula XII, Formula XIII, Formula XIV and Formula XV,

wherein Formula X is

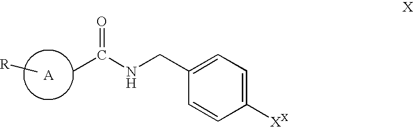

and pharmaceutically acceptable salts thereof; and

wherein ring A is a saturated or unsaturated fused double or triple heterocyclic ring having 1, 2, 3 or 4 heteroatoms selected from group consisting of oxygen, sulfur, or nitrogen; and

wherein R and X are the appropriated substitutents, respectively;

wherein Formula XII is

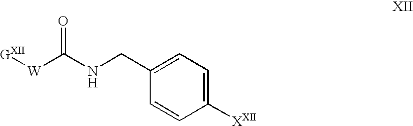

wherein,

X^{XII} is Cl, Br, F, CN or NO₂;

G^{XII} is

a) C₁₋₇alkyl which partially unsaturated and is substituted by hydroxy, or

b) C₁₋₄alkyl substituted by NR^{XII-1}R^{XII-2} or 4-tetrahydropyran;

R^{XII-1} is C₂₋₇alkyl substituted by hydroxy, C₁₋₄alkoxy, aryl^{XII}, or heteroaryl;

R^{XII-2} is hydrogen or C₁₋₇alkyl; or

R^{XII-1} and R^{XII-2} together with the nitrogen to which they are attached form morpholine which may be optionally substituted by ^{XII-}aryl or C₁₋₇alkyl;

W^{XII} is a heterocycle of formula W1^{XII}, W3^{XII}, or W4^{XII};

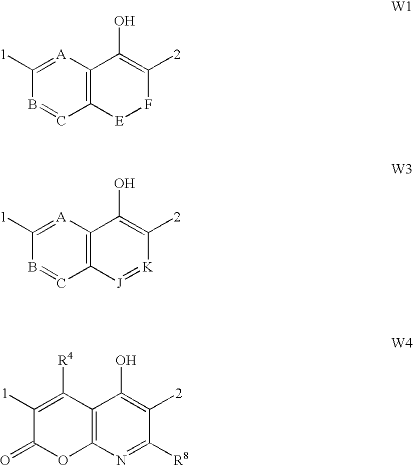

[It is intended that the R⁴ and R⁸ substituents and substituents A-F, J and K on the W1^{XII}, W3^{XII} and W4^{XII} formulas above, as well as the W number under each Formula, are succeeded by the Roman Numeral XII.]

A^{XII} is CR^{XII-4} or nitrogen;

B^{XII} is CR^{XII-5} or nitrogen;

C^{XII} is CR^{XII-6} or nitrogen;

E^{XII} and F^{XII} are such that

a) one is oxygen and the other is C(═O); or

b) E^{XII} is C(═O) and F^{XII} is NR^{XII-7};

J^{XII} and K^{XII} are such that

a) J^{XII} is nitrogen and K^{XII} is CR^{XII-8}; or

b) J^{XII} is CR^{XII-6} and K^{XII} is nitrogen;

with the provisos that when W^{XII} is of formula W3^{XII} and J^{XII} is nitrogen, then at least one of A^{XII} and B^{XII} is nitrogen;

R^{XII-4} is H, halogen, or C₁₋₄alkyl optionally substituted by one to three halogens;

R^{XII-5} is

a) H,

b) halo,

c) OR^{XII-12},

d) SR^{XII-12},

e) C₁₋₇alkyl which may be partially unsaturated and optionally substituted by one or more substituents selected from OR^{XII-12}, SR^{XII-12}, NR^{XII-10}R^{XII-11}, or halo,

f) C₃₋₈cycloalkyl which may be partially unsaturated and is optionally substituted by one or more substituents selected from halogen, OR^{XII-12}, SR^{XII-12}, or NR^{XII-10}R^{XII-11},

g) (C═O)R^{XII-9},

h) S(O)ₘ^{XII}R^{XII-9},

i) (C═O)OR^{XII-2},

j) NHSO₂R^{XII-9},

k) nitro, or

l) cyano;

R^{XII-6} is

a) H,

b) halo,

c) aryl^{XII},

d) het^{XII},

e) OR^{XII-12},

f) SR^{XII-12},

g) C₁₋₇alkyl which may be partially unsaturated and optionally substituted by one or more substituents selected from OR^{XII-12}, SR^{XII-12}, NR^{XII-10}R^{XII-11}, aryl^{XII}, halo, C₃₋₈cycloalkyl optionally substituted by OR¹², or het^{XII} attached through a carbon atom,

h) NR^{XII-10}R^{XII-11},

i) C₃₋₈cycloalkyl which may be partially unsaturated and is optionally substituted by one or more substituents selected from halogen, OR^{XII-12}, SR^{XII-12}, or NR^{XII-10}R^{XII-11},

j) (C═O)R^{XII-9},

k) S(O)ₘ^{XII}R^{XII-9},

l) (C═O)OR^{XII-2},

m) NHSO₂R^{XII-9},

n) nitro, or

o) cyano;

R^{XII-7} is

a) H,

b) C₁₋₇alkyl which may be partially unsaturated and optionally substituted by one or more substituents selected from OR^{XII-12}, SR^{XII-12}, NR^{XII-10}R^{XII-11}, or halo,

c) C₃₋₈cycloalkyl which may be partially unsaturated and is optionally substituted by one or more substituents selected from halogen, OR^{XII-12}, SR^{XII-12}, or NR^{XII-10}R^{XII-11},

d) aryl^{XII}, or

e) het^{XII};

R^{XII-8} is

a) H,

b) C₁₋₇alkyl which may be partially unsaturated and optionally substituted by one or more substituents selected from OR^{XII-12}, SR^{XII-12}, NR^{XII-10}R^{XII-11} or halo,

c) OR^{XII-12}, or

d) SR^{XII-12};

R^{XII-9} is

a) C₁₋₇alkyl,

b) NR^{XII-10}R^{XII-11},

c) aryl^{XII}, or

d) het^{XII}, wherein said het^{XII} is bound through a carbon atom;

R^{XII-10} and R^{XII-11} are independently

a) H,

b) aryl^{XII},

c) C₁₋₇alkyl which may be partially unsaturated and is optionally substituted by one or more substituents selected from CONR²R², CO₂R², het^{XII}, aryl^{XII}, cyano, or halo,

d) C₂₋₇alkyl which may be partially unsaturated and is substituted by one or more substituents selected from NR^{XII-2}R^{XII-2}, OR^{XII-2} or SR^{XII-2},

e) C₃₋₈cycloalkyl which may be partially unsaturated and is optionally substituted by one or more substituents selected from halogen, OR^{XII-2}, SR^{XII-2}, or NR^{XII-2}R^{XII-}2, or

f) R^{XII-10} and R^{XII-11} together with the nitrogen to which they are attached form a het^{XII};

R^{XII-12} is

a) H,

b) aryl

c) het^{XII}

d) C₁₋₇alkyl optionally substituted by aryl^{XII}, het^{XII}, or halogen,

e) C₂₋₇alkyl substituted by OR^{XII-2}, SR^{XII-2}, or NR^{XII-2}R^{XII-2}, or

f) C₃₋₈cycloalkyl which may be partially unsaturated and is optionally substituted by one or more substituents selected from halogen, OR^{XII-1}, SR^{XII-2}, or NR^{XII-2}R^{XII-2};

each m^{XII} is independently 1 or 2;

aryl^{XII} is a phenyl radical or an ortho-fused bicyclic carbocyclic radical wherein at least one ring is aromatic, and aryl^{XII} maybe optionally substituted with one or more substituents selected from halo, OH, cyano, NR^{XII-2}R^{XII-2}, CO₂R^{XII-2}, CF₃, C₁₋₆alkoxy, and C₁₋₆alkyl which maybe further substituted by one to three SR^{XII-2}, NR^{XII-2}R^{XII-2}, OR^{XII-2}, or CO₂R^{XII-2} groups;

het^{XII} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XII} may be optionally substituted with one or more substituents selected from halo, OH, cyano, phenyl, CO₂R^{XII-2}, CF₃, C₁₋₆alkoxy, oxo, oxime, and C₁₋₆ alkyl which may be further substituted by one to three SR^{XII-2}, NR^{XII-2}R^{XII-2}, OR^{XII-2}, or CO₂R^{XII-2} groups;

halo or halogen is F, Cl, Br, I;

1 represents the point of attachment between W^{XII} and G^{XII};

2 represents the point of attachment between WXII and the carbonyl group of Formula (I);

and a pharmaceutically acceptable salt thereof;

wherein Formula XIII is

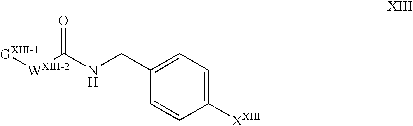

wherein,

X^{XIII} is Cl, Br, F, CN or NO₂;

G^{XIII} is

a) C₃₋₇alkyl which is partially unsaturated and is substituted by hydroxy, or

b) C₁₋₇alkyl substituted by NR^{XIII-1}R^{XII-2} or 4-tetrahydropyran;

R^{XIII-1} is C₂₋₇alkyl substituted by hydroxy, C₁₋₄alkoxy, aryl^{XII}, or heteroaryl^{XII};

R^{XIII-2} is hydrogen or C₁₋₇alkyl; or

R^{XIII-1} and R^{XIII-2} together with the nitrogen to which they are attached form morpholine which may be optionally substituted by aryl^{XII} or C₁₋₇alkyl;

W^{XIII} is

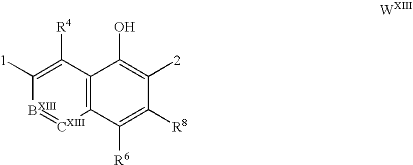

B^{XIII} is CR^{XIII-5} or nitrogen;

C^{XIII} is CR^{XIII-6} or nitrogen;

with the provisos that B^{XIII} and C^{XIII} cannot be both nitrogen;

R^{XIII-4} is H, halogen, or C₁₋₄alkyl optionally substituted by one to three halogens;

R^{XIII-5} is

a) H,

b) halo,

c) OR^{XIII-12},

d) SR^{XIII-12},

e) C₁₋₇alkyl which may be partially unsaturated and optionally substituted by one or more substituents selected from OR^{XII-12}, SR^{XIII-12}, NR^{XIII-10}R^{XIII-11}, or halo,

f) C₃₋₈cycloalkyl which may be partially unsaturated and is optionally substituted by one or more substituents selected from halogen, OR^{XIII-12}, SR^{XIII-12}, or NR^{XIII-10}R^{XIII-11},

g) (C═O)R^{XIII-9},

h) S(O)ₘ^{XIII}R^{XIII-9},

i) (C═O)OR^{XIII-2},

j) NHSO₂R^{XIII-9},

k) nitro, or

l) cyano;

R^{XIII-6} is

a) H,

b) halo,

c) aryl

d) het^{XIII}, or

e) R^{XIII-7};

R^{XIII-7} is

a) OR^{XIII-12},

b) SR^{XIII-12},

c) C₁₋₇alkyl which may be partially unsaturated and optionally substituted by one or more substituents selected from OR^{XIII-12}, SR^{XIII-12}, NR^{XIII-10}R^{XIII-11}, aryl^{XIII}, halo, C₃₋₈cycloalkyl optionally substituted by OR^{XIII-12}, or het^{XIII} attached through a carbon atom,

d) NR^{XIII-10}R^{XIII-11},

e) C₃₋₈cycloalkyl which may be partially unsaturated and is optionally substituted by one or more substituents selected from halogen, OR^{XIII-12}, SR^{XIII-12}, or NR^{XIII-10}R^{XIII-11},

f) (C═O)R^{XIII-9},

g) S(O)ₘ^{XIII}R^{XIII-9},

h) (C═O)OR^{XIII-2},

i) NHSO₂R^{XIII-9},

j) nitro, or

k) cyano;

R^{XIII-8} is

a) H,

b) C₁₋₇alkyl which may be partially unsaturated and optionally substituted by one or more substituents selected from OR^{XIII-12}, SR^{XIII-12}, NR^{XIII-10}R^{XIII-11}, or halo,

c) OR^{XIII-12}, or

d) SR^{XIII-12};

R^{XIII-9} is

a) C₁₋₇alkyl,

b) NR^{XIII-10}R^{XIII-11},

c) aryl^{XIII}, or

d) het^{XIII}, wherein said het^{XIII} is bound through a carbon atom;

R^{XIII-10} and R^{XIII-11} are independently

a) H,

b) aryl^{XIII},

c) C₁₋₇alkyl which may be partially unsaturated and is optionally substituted by one or more substituents selected from CONR^{XIII-2}R^{XIII-2}, CO₂R^{XIII-2}, het^{XIII, aryl}^{XIII}, cyano, or halo,

d) C₂₋₇alkyl which may be partially unsaturated and is substituted by one or more substituents selected from NR^{XIII-2}R², OR^{XIII-2}, or SR^{XIII-2},

e) C₃₋₈cycloalkyl which may be partially unsaturated and is optionally substituted by one or more substituents selected from halogen, OR^{XIII-2}, SR^{XIII-2}, or NR^{XIII-2}R^{XIII-2}, or

f) R^{XIII-10} and R^{XIII-11} together with the nitrogen to which they are attached form a het^{XIII};

R^{XIII-12} is

a) H,

b) aryl^{XIII},

c) het^{XIII},

d) C₁₋₇alkyl optionally substituted by aryl^{XIII}, het^{XIII}, or halogen,

e) C₂₋₇alkyl substituted by OR^{XIII-2}, SR^{XIII-2}, or NR^{XIII-2}R^{XIII-2}, or

f) C₃₋₈cycloalkyl which may be partially unsaturated and is optionally substituted by one or more substituents selected from halogen, OR^{XII-2}, SR^{XIII-2}, or NR^{XIII-2}R^{XIII-2};

each m^{XIII} is independently 1 or 2;

aryl^{XIII} is a phenyl radical or an ortho-fused bicyclic carbocyclic radical wherein at least one ring is aromatic, and aryl^{XIII} maybe optionally substituted with one or more substituents selected from halo, OH, cyano, NR^{XIII-2}R^{XIII-2}, CO₂R^{XIII-2}, CF₃, C₁₋₆alkoxy, and C₁₋₆ alkyl which maybe further substituted by one to three SR^{XIII-2}, NR^{XIII-2}R^{XIII-2} OR^{XIII-2}, or CO₂R^{XIII-2} groups;

het^{XIII} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and ^{XIII-}het may be optionally substituted with one or more substituents selected from halo, OH, cyano, phenyl, CO₂R^{XIII-2}, CF₃, C₁₋₆alkoxy, oxo, oxime, and C₁₋₆ alkyl which may be further substituted by one to three SR^{XIII-2}, NR^{XIII-2}R^{XIII-2}, OR^{XIII-2}, or CO₂R^{XIII-2} groups;

halo or halogen is F, Cl, Br, I;

1 represents the point of attachment between W^{XIII} and G^{XIII};

2 represents the point of attachment between W and the carbonyl group of Formula (I);

and a pharmaceutically acceptable salt thereof;

wherein Formula XIV is

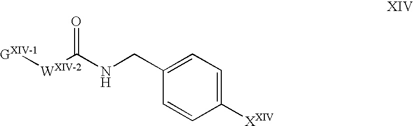

wherein,

X^{XIV} is Cl, Br, F, CN, or NO₂;

G^{XIV} is

a) C₁₋₄alkyl which is fully saturated or partially unsaturated and is substituted by hydroxy, or

b) C₁₋₄alkyl substituted by NR^{XIV-1}R^{XIV-2} or 4-tetrahydropyran;

R^{XIV-1} is C₂₋₇alkyl substituted by hydroxy, C₁₋₄alkoxy, heteroaryl, or aryl^{XIV};

R^{XIV-2} is hydrogen or C₁₋₇alkyl; or

R^{XIV-1} and R^{XIV-2} together with the nitrogen to which they are attached form morpholine which may be optionally substituted by aryl^{XIV} or C₁₋₇alkyl; or pyrrolidine substituted by hydroxy;

W^{XIV} is a heterocycle of formula W1^{XIV}, W2^{XIV}, W3^{XIV}, W4^{XIV}, W5^{XIV}, W6^{XIV}, W7^{XIV}, W8^{XIV}, W9^{XIV}, W10^{XIV}, W11^{XIV}, W12^{XIV}, W13^{XIV}, W14^{XIV}, W15^{XIV}, W16^{XIV}, W17^{XIV}, W18^{XIV}, W19^{XIV}, W20^{XIV}, W21^{XIV} or W22^{XIV}

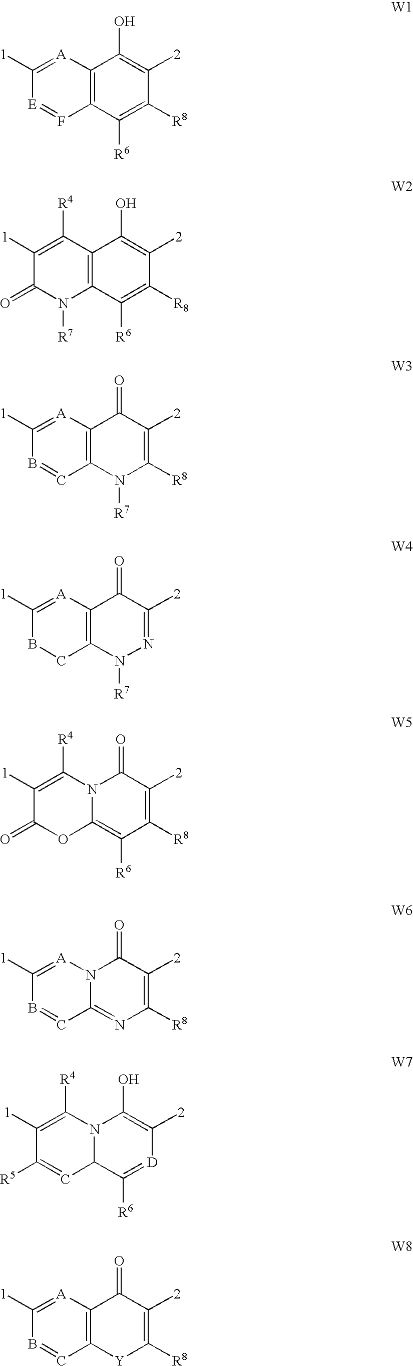

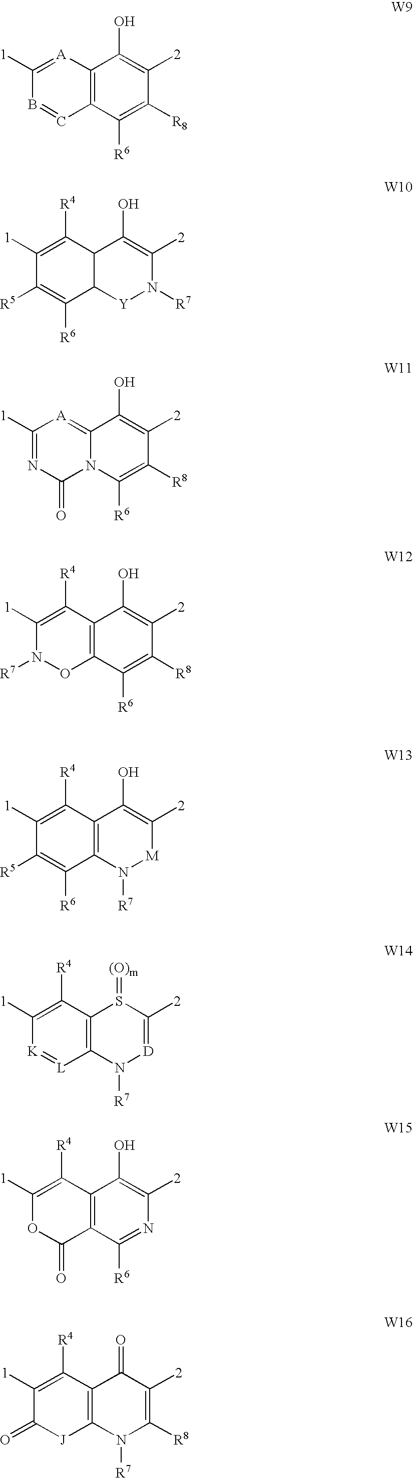

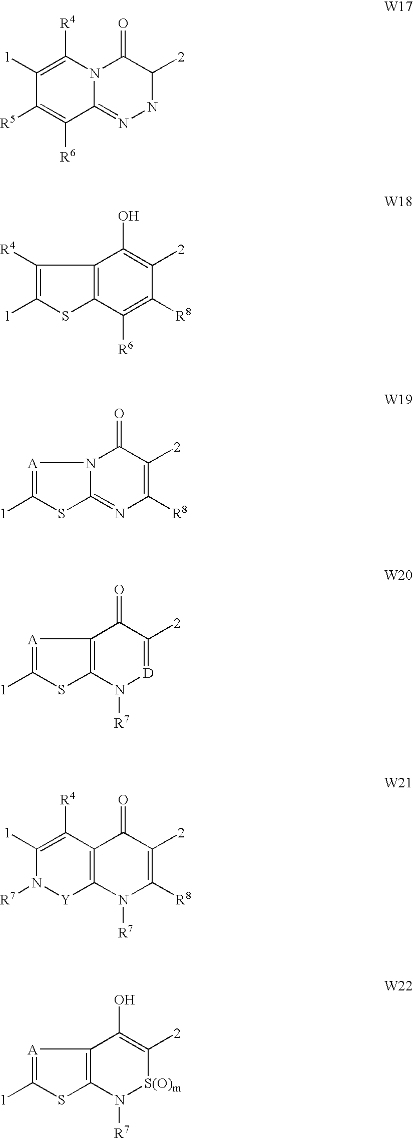

[It is intended that the R substituents and substituents A-F, J, K, L, M and Y on the W Formulas above, as well as the W numbers under the formulas, be succeeded by the Roman Numeral XIV.]

A^{XIV} is CR^{XIV-4} or nitrogen;

B^{XIV} is CR^{XIV-5} or nitrogen;

C^{XIV} is CR^{XIV-6} or nitrogen;

D^{XIV} is CR^{XIV-8} or nitrogen;

E^{XIV} and F are such that one is oxygen and the other is C(═O);

J^{XIV} is NR^{XIV-7} or oxygen;

K^{XIV} and L are defined such that

a) K^{XIV} is CR^{XIV-5} and L^{XIV} is CR^{XIV-6} or

b) K^{XIV} is absent and L^{XIV} is sulfur;

M^{XIV} is oxygen, sulfur, or S(O)ₘ;

Y^{XIV} is oxygen or sulfur;

R^{XIV-4} is H, halogen, or C₁₋₄alkyl optionally substituted by one to three halogens;

R^{XIV-5} is

a) H,

b) halo,

c) OR^{XIV-12},

d) SR^{XIV-12},

e) C₁₋₇alkyl which may be partially unsaturated and optionally substituted by one or more substituents selected from OR^{XIV-12}, SR^{XIV-12}, NR^{XIV-10}R^{XIV-11}, or halo,

f) C₃₋₈cycloalkyl which may be partially unsaturated and is optionally substituted by one or more substituents selected from halogen, OR^{XIV-12}, SR^{XIV-12}, or NR^{XIV-10}R^{XIV-11},

g) (C═O)R^{XIV-9},

h) S(O)ₘ^{XIV}R^{XIV-9},

i) (C═O)OR^{XIV-2},

j) NHSO₂R^{XIV-9},

k) nitro, or

l) cyano;

R^{XIV-6} is

a) H,

b) halo,

c) aryl^{XIV},

d) het^{XIV},

e) OR^{XIV-12},

f) SR^{XIV-12},

g) C₁₋₇alkyl which may be partially unsaturated and optionally substituted by one or more substituents selected from OR^{XIV-12}, SR^{XIV-12}, NR^{XIV-10}R^{XIV-11}, ^{XIV-}aryl, halo, C₃₋₈cycloalkyl optionally substituted by OR^{XIV-12}, or ^{XIV-}het attached through a carbon atom,

h) NR^{XIV-10}R^{XIV-11},

i) C₃₋₈cycloalkyl which may be partially unsaturated and is optionally substituted by one or more substituents selected from halogen, OR^{XIV-12}, SR^{XIV-12}, or NR^{XIV-10}R^{XIV-11},

j) (C═O)R^{XIV-9},

k) S(O)ₘ^{XIV}R^{XIV-9},

l) (C═O)OR^{XIV-2},

m) NHSO₂R^{XIV-9},

n) nitro, or

o) cyano;

R^{XIV-7} is

a) H,

b) C₁₋₇alkyl which may be partially unsaturated and optionally substituted by one or more substituents selected from OR^{XIV-12}, SR^{XIV-12}, NR^{XIV-10}R^{XIV-11}, or halo,

c) C₃₋₈cycloalkyl which may be partially unsaturated and is optionally substituted by one or more substituents selected from halogen, OR^{XIV-12}, SR^{XIV-12}, or NR^{XIV-10}R^{XIV-11},

d) aryl^{XIV}, or

e) het^{XIV};

R^{XIV-8} is

a) H,

b) C₁₋₇alkyl which may be partially unsaturated and optionally substituted by one or more substituents selected from OR^{XIV-12}, SR^{XIV-12}, NR^{XIV-10}R^{XIV-11}, or halo,

c) OR^{XIV-12}, or

d) SR^{XIV-12};

R^{XIV-9} is

a) C₁₋₇alkyl,

b) NR^{XIV-10}R^{XIV-11},

c) aryl^{XIV}, or

d) het^{XIV} wherein said ^{XIV-}het is bound through a carbon atom;

R^{XIV-10} and R^{XIV-11} are independently

a) H,

b) aryl^{XIV},

c) C₁₋₇alkyl which may be partially unsaturated and is optionally substituted by one or more substituents selected from CONR^{XIV-2}R^{XIV-2}, CO₂R^{XIV-2}, het^{XIV}, aryl^{XIV}, cyano, or halo,

d) C₂₋₇alkyl which may be partially unsaturated and is substituted by one or more substituents selected from NR^{XIV-2}R^{XIV-2}, OR^{XIV-2}, or SR^{XIV-2},

e) C₃₋₈cycloalkyl which may be partially unsaturated and is optionally substituted by one or more substituents selected from halogen, OR^{XIV-2}, SR^{XIV-2}, or NR^{XIV-2}R^{XIV-2}, or

f) R^{XIV-10} and R^{XIV-11} together with the nitrogen to which they are attached form a het^{XIV};

R^{XIV-12} is

a) H,

b) aryl^{XIV},

c) het^{XIV}

d) C₁₋₇alkyl optionally substituted by aryl^{XIV}, or halogen,

e) C₂₋₇alkyl substituted by OR^{XIV-2}, SR^{XIV-2}, or NR^{XIV-2}R^{XIV-2}, or

f) C₃₋₈cycloalkyl which may be partially unsaturated and is optionally substituted by one or more substituents selected from halogen, OR^{XIV-2}, SR^{XIV-2}, or NR^{XIV-2}R^{XIV-2};

each m^{XIV} is independently 1 or 2;

aryl^{XIV} is a phenyl radical or an ortho-fused bicyclic carbocyclic radical wherein at least one ring is aromatic, and aryl^{XIV} may be optionally substituted with one or more substituents selected from halo, OH, cyano, NR^{XIV-2}R^{XIV-2}, CO₂R^{XIV-2}, CF₃, C₁₋₆alkoxy, and C₁₋₆ alkyl which maybe further substituted by one to three SR², NR^{XIV-2}R^{XIV-2}, OR^{XIV-2}, or CO₂R^{XIV-2} groups;

het^{XIV} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XIV} may be optionally substituted with one or more substituents selected from halo, OH, cyano, phenyl, CO₂R^{XIV-2}, CF₃, C₁₋₆alkoxy, oxo, oxime, and C₁₋₆ alkyl which may be further substituted by one to three SR^{XIV-2}, NR^{XIV-2}R^{XIV-2}, OR^{XIV-2}, or CO₂R^{XIV-2} groups;

halo or halogen is F, Cl, Br, I;

1 represents the point of attachment between W and G;

2 represents the point of attachment between W and the carbonyl group of Formula (I);

and a pharmaceutically acceptable salt thereof;

wherein Formula XV is

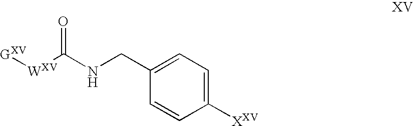

or a pharmaceutically acceptable salt thereof,

wherein,

X^{XV} is Cl, Br, F, CN or NO₂;

G^{XV} is

a) C₁₋₄alkyl which is fully saturated or partially unsaturated and is substituted by hydroxy, or

b) C₁₋₄alkyl substituted by NR^{XV-1}R^{XV-2} or 4-tetrahydropyran;

R^{XV-1} is C₂₋₇alkyl substituted by hydroxy, C₁₋₄alkoxy, or aryl;

R^{XV-2} is hydrogen or C₁₋₇alkyl; or

R^{XV-1} and R^{XV-2} together with the nitrogen to which they are attached form

a) a morpholine which may be optionally substituted by aryl or C₁₋₇alkyl; or

b) a pyrrolidine ring substituted by hydroxy;

W^{XV} is a heterocycle of formula W1^{XV}, W2^{XV}, W3^{XV}, W4^{XV}, W5^{XV}, W6^{XV}, W7^{XV} or W8^{XV}

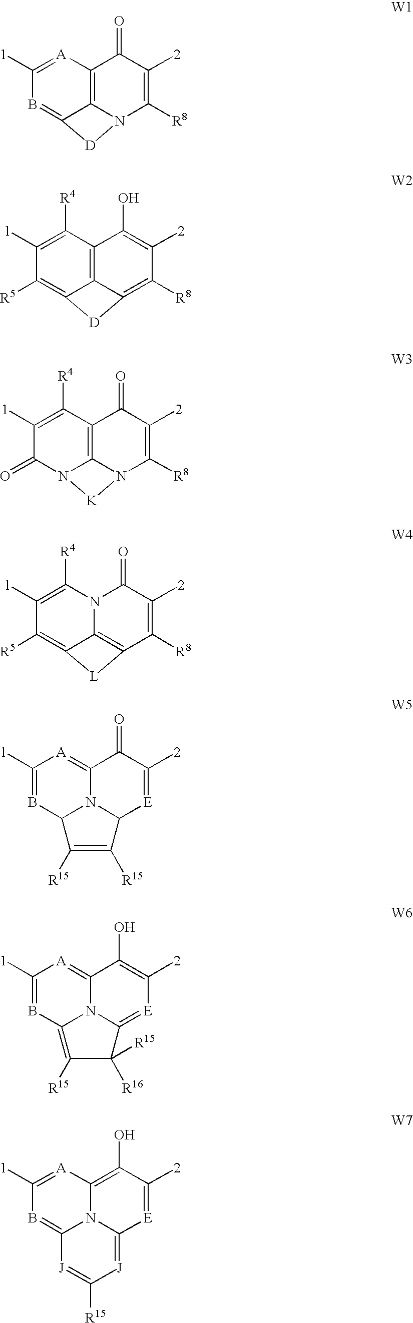

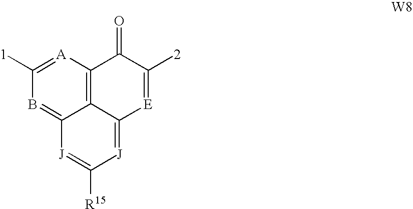

[It is intended that the R substituents and substituents A, B, D, E, J, K and L on the W^{XV-1} to W^{XV-8} Formula above, as well as the W numbers under the formulas, be succeeded by the Roman Numeral XV.]

A^{XV} is CR^{XV-4} or nitrogen;

B^{XV} is CR^{XV-5} or nitrogen;

D^{XV} is

a) —(CR¹³R^{XV-14})ₐ^{XV}—, where a^{XV} is 2 or 3

b) —(CR¹⁵R^{XV-16})₄—,

c) —Y^{XV}—CR¹³R^{XV-14}—CR¹³R^{XV-14}—,

d) —CR¹³R^{XV-14}—Y^{XV}—CR¹³R^{XV-14}—,

e) —Y^{XV}—CR¹³R^{XV-14}—Y^{XV}—,

f) —CR¹³R^{XV-14}—CR¹³R^{XV-14}—Y^{XV}—,

g) —Y^{XV}—(CR¹⁵R^{XV-16})ₙ^{XV}—,

h) —Y^{XV}—CR¹⁵═CR¹⁵—,

i) —Y^{XV}—CR¹⁵═N—,

j) —CR¹⁵═CR¹⁵—Y^{XV}—,

k) —N═CR¹⁵—Y^{XV}—,

l) —(CR¹⁵R^{XV-16})_{b}^{XV}—N═CR¹⁵—, where b^{XV} is 0 or 1

m) —CR¹⁵═N—(CR¹⁵R^{XV-16})_{b}^{XV}—, where b^{XV} is 0 or 1

n) —N═N—,

o) —N═CR¹⁵—(CR¹⁵R¹⁶)_{b}^{XV}, where b^{XV} is 0 or 1

p) —CR¹⁵═CR¹⁵—,

q) —N═N—Y^{XV},

r) —Y^{XV}N═N—,

s) —Y^{XV}—N═CR¹⁵, or

t) —CR¹⁵═R^{XV-16}—Y^{XV}—CR¹⁵R^{XV-16}—CR¹⁵R^{XV-16};

E^{XV} is CR⁸ or nitrogen;

J^{XV} is CR¹⁵ or nitrogen;

K^{XV} is

a) —(CR¹⁵R^{XV-16})ₐ^{XV}—, where a^{XV} is 2 or 3, or

b) —CR¹⁵═CR¹⁵;

L^{XV} is

a) —(CR¹⁵R^{XV-16})ₐ^{XV}, where a^{XV} is 2 or 3, or

b) —Y^{XV}—(CR¹⁵R^{XV-16})—(CR¹⁵R^{XV-16})—;

Y^{XV} is oxygen, S(O)ₘ^{XV}, or NR^{XV-7};

with the provisos that:

when W^{XV} is of formula W^{XV}1;

G^{XV} is C₁₋₄alkyl which is fully saturated and is substituted by hydroxy or morpholinyl, in which morpholinyl is attached through nitrogen;

A^{XV} is CR^{XV-4}; B^{XV} is CR^{XV-5}; and

R_{XV-8} is hydrogen then at least one of R^{XV-13}, R^{XV-14}, or R^{XV-7} is not hydrogen or C₁₋₇alkyl;

when W^{XV} is of formula W^{XV}1, A^{XV} is CR^{XV-4}, B^{XV} is CR^{XV-5}, D^{XV} is —Y^{XV}—CR¹³R^{XV-14}—CR¹³R^{XV-14}—, and R^{XV-8} is hydrogen then Y^{XV} is not oxygen;

when W^{XV} is of formula W^{XV}1, A^{XV} is CR^{XV-4}, and B^{XV} is CR^{XV-5} then D^{XV} is not —CR¹⁵═CR¹⁵;

R^{XV-4} is H, halogen, or C₁₋₄alkyl optionally substituted by one to three halogens;

R^{XV-5} is

a) H,

b) halo,

c) OR^{XV-12},

d) SR^{XV-12},

e) C₁₋₇alkyl which may be partially unsaturated and optionally substituted by one or more substituents selected from OR^{XV-12}, SR^{XV-12}, NR^{XV-10}R^{XV-11}, or halo,

f) C₃₋₈cycloalkyl which may be partially unsaturated and is optionally substituted by one or more substituents selected from halogen, OR^{XV-12}, SR^{XV-12}, or NR^{XV-10}R^{XV-11},

g) (C═O)R^{XV-9},

h) S(O)ₘ^{XV}R^{XV-9},

i) (C═O)OR^{XV-2},

j) NHSO₂R^{XV-9},

k) nitro, or

l) cyano;

R^{XV-7} is

a) H,

b) C₁₋₇alkyl which may be partially unsaturated and optionally substituted by one or more substituents selected from OR^{XV-12}, SR^{XV-12}, NR^{XV-10}R^{XV-11}, or halo,

c) C₃₋₈cycloalkyl which may be partially unsaturated and is optionally substituted by one or more substituents selected from halogen, OR^{XV-12}, SR^{XV-12}, or NR^{XV-10}R^{XV-11},

d) aryl^{XV},

e) het^{XV},

f) (C═O)R^{XV-9}, or

g) S(O)ₘ^{XV}R^{XV-9};

R^{XV-8} is

a) H,

b) C₁₋₇alkyl which may be partially unsaturated and optionally substituted by one or more substituents selected from OR^{XV-12}, SR^{XV-12}, NR^{XV-10}R^{XV-11}, or halo,

c) OR^{XV-12}, or

d) SR^{XV-12};

R^{XV-9} is

a) C₁₋₇alkyl optionally substituted by OR^{XV-12} or NR^{XV-2}R^{XV-2},

b) C₃₋₈cycloalkyl optionally substituted by OR^{XV-12} or NR^{XV-2}R^{XV-2},

c) NR^{XV-10}R^{XV-11},

d) aryl^{XV}, or

e) het^{XV}, wherein said het^{XV} is bound through a carbon atom;

R^{XV-10} and R^{XV-11} are independently

a) H,

b) aryl^{XV},

c) C₁₋₇alkyl which may be partially unsaturated and is optionally substituted by one or more substituents selected from CONR²R^{XV-2}, CO₂R^{XV-2}, het^{XV}, aryl^{XV}, cyano, or halo,

d) C₂₋₇alkyl which may be partially unsaturated and is substituted by one or more substituents selected from NR^{XV-2}R^{XV-2}, OR^{XV-2}, or SR^{XV-2},

e) C₃₋₈cycloalkyl which may be partially unsaturated and is optionally substituted by one or more substituents selected from halogen, OR^{XV-2}, SR^{XV-2}, or NR^{XV-2}R^{XV-2}, or

f) R^{XV-10} and R^{XV-11} together with the nitrogen to which they are attached form a het^{XV};

R^{XV-12} is

a) H,

b) aryl^{XV},

c) het^{XV},

d) C₁₋₇alkyl optionally substituted by aryl^{XV}, or halogen,

e) C₂₋₇alkyl substituted by OR^{XV-2}, SR^{XV-2}, or NR^{XV-2}R^{XV-2}, or

f) C₃₋₈cycloalkyl which may be partially unsaturated and is optionally substituted by one or more substituents selected from halogen, OR^{XV-2}, SR^{XV-2}, or NR^{XV-2}R^{XV-2};

R^{XV-13}, R^{XV-14}, R^{XV-15}, and R^{XV-16} are independently

a) H

b) C₁₋₇alkyl which may be partially unsaturated and optionally substituted by one or more OR^{XV-12}, SR^{XV-12}, NR^{XV-10}R^{XV-11}, or halo groups,

c) C₃₋₈cycloalkyl which may be partially unsaturated and is optionally substituted by one or more substituents selected from halogen,

d) aryl^{XV},

e) het^{XV}, wherein said het^{XV} is bound through a carbon atom,

f) OR^{XV-12},

g) SR^{XV-12},

h) NR^{XV-10}R^{XV-11};

i) (C═O)OR^{XV-2}, or

j) R¹³ and R¹⁴ or R¹⁵ and R¹⁶ together with the carbon to which they are attached form (C═O);

each m^{XV} is independently 0, 1 or 2;

each n^{XV} is independently 1 or 3;

aryl^{XV} is a phenyl radical or an ortho-fused bicyclic carbocyclic radical wherein at least one ring is aromatic, and aryl^{XV} may be optionally substituted with one or more substituents selected from halo, OH, cyano, NR^{XV-2}R^{XV-2}, CO₂R^{XV-2}, CF₃, C₁₋₆alkoxy, and C₁₋₆ alkyl which maybe further substituted by one to three SR^{XV-2}, NR^{XV-2}R^{XV-2}, OR^{XV-2}, or CO₂R^{XV-2} groups;

het^{XV} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XV} may be optionally substituted with one or more substituents selected from halo, OH, cyano, phenyl, CO₂R^{XV-2}, CF₃, C₁₋₆alkoxy, oxo, oxime, and C₁₋₆ alkyl which may be further substituted by one to three SR^{XV-2}, NR^{XV-2}R^{XV-2}, OR^{XV-2}, or CO₂R^{XV-2} groups;

halo or halogen is F, Cl, Br, I;

1 represents the point of attachment between W^{XV} and G^{XV};

2 represents the point of attachment between WXV and the carbonyl group of Formula (I);

and pharmaceutically acceptable salts thereof.

Also provided is the use of compounds of Formula X and XII-XV to prepare medicaments for preventing or treating atherosclerosis or reestenosis in mammals.

The advantage of using compounds of Formula I and II in the method of our invention is their extensive activity against herpesviruses since atherosclerosis is related to the number of herpesvirus infections. Drugs containing compound of Formula II could prevent the inflammatory response resulting from reactivation of HCMV, EBV, HSV-1, HSV-2, HHV-8 and VZV.

### DETAILED DESCRIPTION OF THE INVENTION

The compounds of Formula X, their method of preparation and formulation into pharmaceutical dosage forms are described in U.S. patent application Ser. No. 09/904,065, filed Jul. 12, 2001. The disclosure of U.S. patent application Ser. No. 09/904,065 is herein incorporated in its entirety by reference.

The compounds of Formula XII, their method of preparation and formulation into pharmaceutical dosage forms are described in U.S. patent application Ser. No. 09/887,620, filed Jun. 22, 2001. The disclosure of U.S. patent application Ser. No. 09/887,620 is herein incorporated in its entirety by reference.

The compounds of Formula XIII, their method of preparation and formulation into pharmaceutical dosage forms are described in U.S. patent application Ser. No. 09/887,226, filed Jun. 22, 2001. The disclosure of U.S. patent application Ser. No. 09/887,226 is herein incorporated in its entirety by reference.

The compounds of Formula XIV, their method of preparation and formulation into pharmaceutical dosage forms are described in U.S. patent application Ser. No. 09/887,794, filed Jun. 22, 2001. The disclosure of U.S. patent application Ser. No. 09/887,794 is herein incorporated in its entirety by reference.

The compounds of Formula XV, their method of preparation and formulation into pharmaceutical dosage forms are described in U.S. patent application Ser. No. 09/887,578, filed Jun. 22, 2001. The disclosure of U.S. patent application Ser. No. 09/887,578 is herein incorporated in its entirety by reference.

The correspondence between the compounds utilized in the method of the invention and the compounds incorporated by reference is as follows:

Formula X corresponds to Formula I of U.S. patent application Ser. No. 09/904,065.

Formula XII corresponds to Formula I of U.S. patent application Ser. No. 09/887,620.

Formula XIII corresponds to Formula I of U.S. patent application Ser. No. 09/887,226.

Formula XIV corresponds to Formula I of U.S. patent application Ser. No. 09/887,794.

Formula XV corresponds to Formula I of U.S. patent application Ser. No. 09/887,578.

The invention further provides a method for preventing or treating atherosclerosis or restenosis in mammals, wherein the compound administered has the Formula X and is selected from the group consisting of compound 1 to 16:

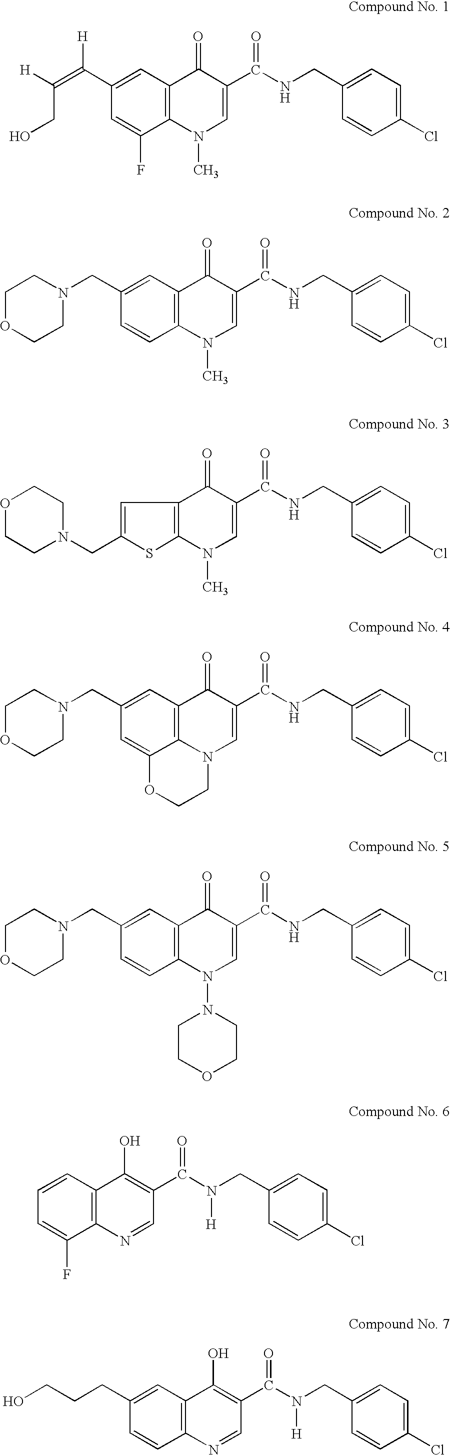

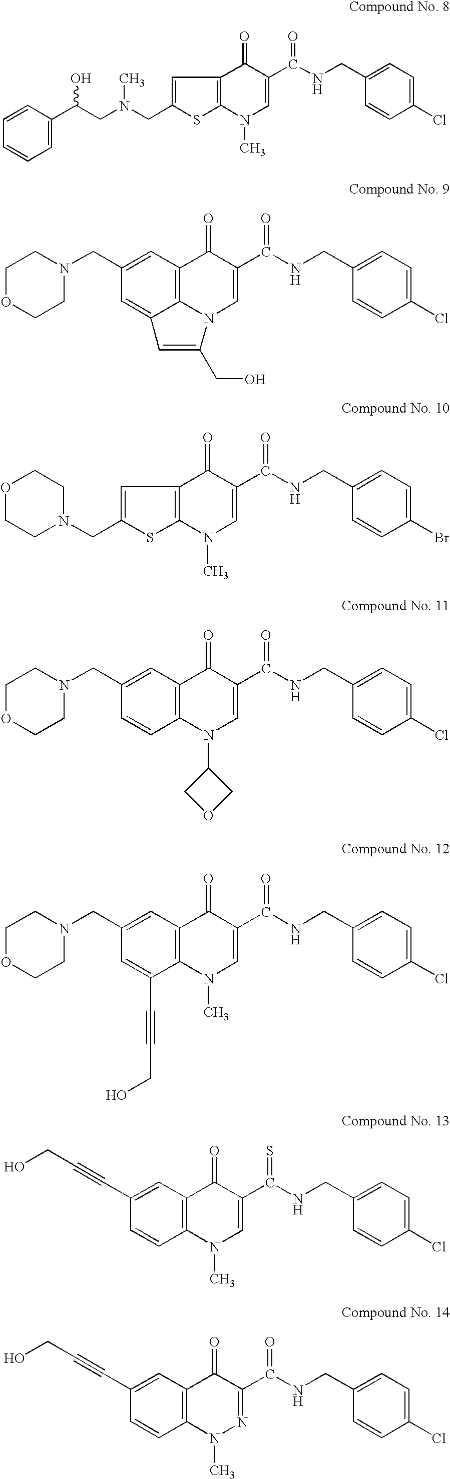

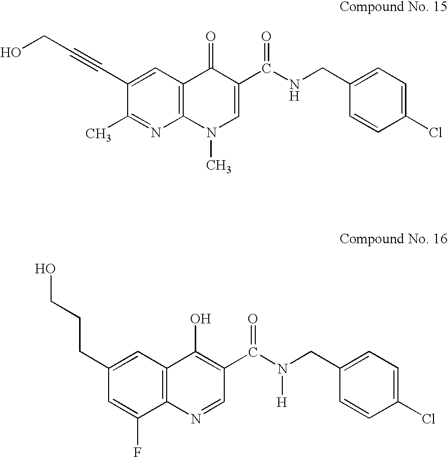

A method of selecting compounds of Formula X that are useful for preventing or treating atherosclerosis or restinosis in a mammal comprising:

a) measuring IC₅₀ of the method of interest that inhibits a wild type herpes virus,

b) measuring IC₅₀ of the same compound that inhibits a binding domain mutant herpes virus which is the same strain as the wild type herpes virus,

c) comparing IC₅₀ of step a with IC₅₀ of step b; and

d) selecting the compound of Formula X of interest wherein the IC₅₀ of step b is at least 3 times greater than the IC₅₀ of step a.

A method of selecting compounds of Formula X that are useful for preventing or treating atherosclerosis or restenosis in a mammal comprising:

a) measuring IC₅₀ of The method of interest that inhibits a binding domain mutant herpes virus,

b) measuring IC₅₀ of the same compound that inhibits a wild type herpes virus which is the same strain as the mutant herpes virus,

c) comparing IC₅₀ of step a with IC₅₀ of step b; and

d) selecting the compound of Formula X of interest wherein the IC₅₀ of step a is at least 3 times greater than the IC₅₀ of step b.

A method of selecting compounds of Formula X that are useful for preventing or treating atherosclerosis or restenosis in a mammal, comprising:

a) measuring IC₅₀ of the method of interest that inhibits a wild type HSV-1,

b) measuring IC₅₀ of the same compound that inhibits a binding domain mutant HSV-1 which is the same strain as the wild type herpes virus,

c) comparing IC₅₀ of step a with IC₅₀ of step b; and

d) selecting the compound of Formula X of interest wherein the IC₅₀ of step b is at least 3 times greater than the IC₅₀ of step a.

A method of selecting compounds of Formula X that are useful for preventing or treating atherosclerosis or restenosis in a mammal, comprising:

a) measuring IC₅₀ of the method of interest that inhibits a binding domain mutant HSV-1,

b) measuring IC₅₀ of the same compound of Formula X that inhibits a wild type herpes virus which is the same strain as the mutant HSV-1,

c) comparing IC₅₀ of step a with IC₅₀ of step b; and

d) selecting the compound of Formula X of interest wherein the IC₅₀ of step a is at least 3 times greater than the IC₅₀ of step b.

A method of selecting compounds of Formula X that are useful for preventing or treating atherosclerosis or restenosis in a mammal, comprising:

a) measuring IC₅₀ of the method of interest that inhibits a wild type HSV-1,

b) measuring IC₅₀ of the same compound that inhibits a binding domain mutant HSV-1 which is the same strain as the wild type herpes virus,

c) comparing IC₅₀ of step a with IC₅₀ of step b; and

d) selecting the compound of Formula X of interest wherein the IC₅₀ of step b is at least 3 times greater than the IC₅₀ of step a,

wherein HSV-1 is HSV-1 KOS, HSV-1 F, HSV-1 DJL or HSV-1-Patton.

A method of selecting compounds of Formula X that are useful for preventing or treating atherosclerosis or restenosis in a mammal, comprising:

a) measuring IC₅₀ of the method of interest that inhibits a binding domain mutant HSV-1,

b) measuring IC₅₀ of the same compound of Formula X that inhibits a wild type herpes virus which is the same strain as the mutant HSV-1,

c) comparing IC₅₀ of step a with IC₅₀ of step b; and

d) selecting the compound of interest wherein the IC₅₀ of step a is at least 3 times greater than the IC₅₀ of step b; and

wherein HSV-1 is HSV-1 KOS, HSV-1 F, HSV-1 DJL or HSV-1-Patton.

A method of selecting compounds of Formula X that are useful for preventing or treating atherosclerosis or restinosis in a mammal, comprising:

a) measuring IC₅₀ of the method of interest that inhibits a binding domain mutant HSV-1,

b) measuring IC₅₀ of the same compound of Formula X that inhibits a wild type herpes virus which is the same strain as the mutant HSV-1,

c) comparing IC₅₀ of step a with IC₅₀ of step b; and

d) selecting the compound of Formula X of interest wherein the IC₅₀ of step a is at least 3 times greater than the IC₅₀ of step b,

wherein the mutation of a wild type herpes virus to mutant herpes virus is at amino acid 823 from valine to alanine.

A method of selecting compounds of Formula X that are useful for preventing or treating atherosclerosis or restenosis in a mammal, comprising:

a) measuring IC₅₀ of the method of interest that inhibits a wild type HSV-1,

b) measuring IC₅₀ of the same compound that inhibits a binding domain mutant HSV-1 which is the same strain as the wild type herpes virus,

c) comparing IC₅₀ of step a with IC₅₀ of step b, and

d) selecting the compound of interest wherein the IC₅₀ of step b is at least 3 times greater than the IC₅₀ of step a,

wherein HSV-1 is HSV-1 KOS, HSV-1 F, HSV-1 DJL or HSV-1-Patton and wherein the mutation of a wild type herpes virus to mutant herpes virus as at amino acid 823 from valine to alanine.

A method of selecting compounds of Formula X that are useful for preventing or treating atherosclerosis or restenosis in a mammal, comprising:

a) measuring IC₅₀ of the method of interest that inhibits a wild type HSV-2,

b) measuring IC₅₀ of the same compound that inhibits a binding domain mutant HSV-2 which is the same strain as the wild type herpes virus,

c) comparing IC₅₀ of step a with IC₅₀ of step b, and

d) selecting the compound of Formula X of interest wherein the IC₅₀ of step b is at least 3 times greater than the IC₅₀ of step a.

A method of selecting compounds of Formula X that are useful for preventing or treating atherosclerosis or restenosis in a mammal, comprising:

a) measuring IC₅₀ of the method of interest that inhibits a binding domain mutant HSV-2,

b) measuring IC₅₀ of the same compound of Formula X that inhibits a wild type herpes virus which is the same strain as the mutant HSV-2,

c) comparing IC₅₀ of step a with IC₅₀ of step b, and

d) selecting the compound of Formula X of interest wherein the IC₅₀ of step a is at least 3 times greater than the IC₅₀ of step b.

A method of selecting compounds of Formula X that are useful for preventing or treating atherosclerosis or restenosis in a mammal, comprising:

a) measuring IC₅₀ of the method of interest that inhibits a binding domain mutant HSV-1,

b) measuring IC₅₀ of the same compound of Formula X that inhibits a wild type herpes virus which is the same strain as the mutant HSV-1,

c) comparing IC₅₀ of step a with IC₅₀ of step b, and

d) selecting the compound of Formula X of interest wherein the IC₅₀ of step a is at least 3 times greater than the IC₅₀ of step b,

wherein the mutation of a wild type herpes virus to mutant herpes virus is at amino acid 823 from valine to alanine and wherein HSV-2 is HSV-2 MS, HSV-2 35D, or HSV-2 186.

A method of selecting compounds of Formula X that are useful for preventing or treating atherosclerosis or restenosis in a mammal, comprising:

a) measuring IC₅₀ of the method of interest that inhibits a wild type HSV-2,

b) measuring IC₅₀ of the same compound that inhibits a binding domain mutant HSV-2 which is the same strain as the wild type herpes virus,

c) comparing IC₅₀ of step a with IC₅₀ of step b; and

d) selecting the compound of interest of Formula X wherein the IC₅₀ of step b is at least 3 times greater than the IC₅₀ of step a,

wherein HSV-2 is HSV-2 MS, HSV-2 35D, or HSV-2 186.

A method of selecting compounds of Formula X that are useful for preventing or treating atherosclerosis or restenosis in a mammal, comprising:

a) measuring IC₅₀ of the method of interest that inhibits a wild type HCMV,

b) measuring IC₅₀ of the same compound of Formula X that inhibits a binding domain mutant HCMV which is the same strain as the wild type herpes virus,

c) comparing IC₅₀ of step a with IC₅₀ of step b; and

d) selecting the compound of Formula X of interest wherein the IC₅₀ of step b is at least 3 times greater than the IC₅₀ of step a.

A method of selecting compounds of Formula X that are useful for preventing or treating atherosclerosis or restenosis in a mammal, comprising:

a) measuring IC₅₀ of the method of interest that inhibits a binding domain mutant HCMV,

b) measuring IC₅₀ of the same compound of Formula X that inhibits a wild type herpes virus which is the same strain of the mutant HCMV,

c) comparing IC₅₀ of step a with IC₅₀ of step b; and

d) selecting the compound of Formula X of interest wherein the IC₅₀ of step a is at least 3 times greater than the IC₅₀ of step b.

A method of selecting compounds of Formula X that are useful for preventing or treating atherosclerosis or restenosis in a mammal, comprising:

a) measuring IC₅₀ of the method of interest that inhibits a wild type HSV-2,

b) measuring IC₅₀ of the same compound that inhibits a binding domain mutant HSV-2 which is the same strain as the wild type herpes virus,

c) comparing IC₅₀ of step a with IC₅₀ of step b, and

d) selecting the compound of Formula X of interest wherein the IC₅₀ of step b is at least 3 times greater than the IC₅₀ of step a,

wherein HCMV is AD169.

A method of selecting compounds of Formula X that are useful for preventing or treating atherosclerosis or restenosis in a mammal, comprising:

a) measuring IC₅₀ of the method of interest that inhibits a binding domain mutant HSV-2,

b) measuring IC₅₀ of the same compound of Formula X that inhibits a wild type herpes virus which is the same strain as the mutant HSV-2,

c) comparing IC₅₀ of step a with IC₅₀ of step b; and

d) selecting the compound of Formula X of interest wherein the IC₅₀ of step a is at least 3 times greater than the IC₅₀ of step b,

wherein HCMV is AD169.

A method of selecting compounds of Formula X that are useful for preventing or treating atherosclerosis or restinosis in a mammal comprising:

a) measuring IC₅₀ of the method of interest that inhibits a wild type herpes virus,

b) measuring IC₅₀ of the same compound that inhibits a binding domain mutant herpes virus which is the same strain as the wild type herpes virus,

c) comparing IC₅₀ of step a with IC₅₀ of step b, and

d) selecting the compound of Formula X of interest wherein the IC₅₀ of step b is at least 3 times greater than the IC₅₀ of step a,

wherein IC₅₀ of step b is at least 5 times greater than the IC₅₀ of step a.

A method of selecting compounds of Formula X that are useful for preventing or treating atherosclerosis or restenosis in a mammal, comprising:

a) measuring IC₅₀ of the method of interest that inhibits a wild type HSV-1,

b) measuring IC₅₀ of the same compound that inhibits a binding domain mutant HSV-1 which is the same strain as the wild type herpes virus,

c) comparing IC₅₀ of step a with IC₅₀ of step b, and

d) selecting the compound of Formula X of interest wherein the IC₅₀ of step b is at least 3 times greater than the IC₅₀ of step a,

wherein IC₅₀ of step b is at least 5 times greater than the IC₅₀ of step a.

A method of selecting compounds of Formula X that are useful for preventing or treating atherosclerosis or restenosis in a mammal, comprising:

a) measuring IC₅₀ of the method of interest that inhibits a wild type HSV-2,

b) measuring IC₅₀ of the same compound that inhibits a binding domain mutant HSV-2 which is the same strain as the wild type herpes virus,

c) comparing IC₅₀ of step a with IC₅₀ of step b, and

d) selecting the compound of Formula X of interest wherein the IC₅₀ of step b is at least 3 times greater than the IC₅₀ of step a,

wherein IC₅₀ of step b is at least 5 times greater than the IC₅₀ of step a.

A method of selecting compounds of Formula X that are useful for preventing or treating atherosclerosis or restenosis in a mammal, comprising:

a) measuring IC₅₀ of the method of interest that inhibits a wild type HCMV,

b) measuring IC₅₀ of the same compound of Formula X that inhibits a binding domain mutant HCMV which is the same strain as the wild type herpes virus,

c) comparing IC₅₀ of step a with IC₅₀ of step b, and

d) selecting the compound of Formula X of interest wherein the IC₅₀ of step b is at least 3 times greater than the IC₅₀ of step a,

wherein IC₅₀ of step b is at least 5 times greater than the IC₅₀ of step a.

A method for preventing or treating atherosclerosis or restenosis in mammals, wherein the compound administered has the Formula XII and wherein W^{XII} is of the formula W^{XII}W1.

A method for preventing or treating atherosclerosis or restenosis in mammals, wherein the compound administered has the Formula XII and wherein W^{XII} is of the formula W^{XII}W1.1.

A method for preventing or treating atherosclerosis or restenosis in mammals, wherein the compound administered has the Formula XII and wherein W^{XII} is of the formula W^{XII}1.2.

A method for preventing or treating atherosclerosis or restenosis in mammals, wherein the compound administered has the Formula XII and wherein W^{XII} is of the formula W^{XII}1.3.

A method for preventing or treating atherosclerosis or restenosis in mammals, wherein the compound administered has the Formula XII and wherein W^{XII} is of the formula W^{XII}1.4.

A method for preventing or treating atherosclerosis or restenosis in mammals, wherein the compound administered has the Formula XII and wherein W^{XII} is of the formula W^{XII}1.5.

A method for preventing or treating atherosclerosis or restenosis in mammals, wherein the compound administered has the Formula XII and wherein W^{XII} is of the formula W^{XII}1.6.

A method for preventing or treating atherosclerosis or restenosis in mammals, wherein the compound administered has the Formula XII and wherein W^{XII} is of the formula W^{XII}1.7.

A method for preventing or treating atherosclerosis or restenosis in mammals, wherein the compound administered has the Formula XII and wherein W^{XII} is of the formula W^{XII}1.8.

A method for preventing or treating atherosclerosis or restenosis in mammals, wherein the compound administered has the Formula XII and wherein W^{XII} is of the formula W^{XII}1.9.

A method for preventing or treating atherosclerosis or restenosis in mammals, wherein the compound administered has the Formula XII and wherein W^{XII} is of the formula W^{XII}1.10.

A method for preventing or treating atherosclerosis or restenosis in mammals, wherein the compound administered has the Formula XII and wherein W^{XII} is of the formula W^{XII}1.11.

A method for preventing or treating atherosclerosis or restenosis in mammals, wherein the compound administered has the Formula XII and wherein W^{XII} is of the formula W^{XII}1.12.

A method for preventing or treating atherosclerosis or restenosis in mammals, wherein the compound administered has the Formula XII and wherein W^{XII} is of the formula W^{XII}1.13.

A method for preventing or treating atherosclerosis or restenosis in mammals, wherein the compound administered has the Formula XII and wherein W^{XII} is of the formula W^{XII}1.14.

A method for preventing or treating atherosclerosis or restenosis in mammals, wherein the compound administered has the Formula XII and wherein W^{XII} is of the formula W^{XII}1.15.

A method for preventing or treating atherosclerosis or restenosis in mammals, wherein the compound administered has the Formula XII and wherein W^{XII} is of the formula W^{XII}1.16.

A method for preventing or treating atherosclerosis or restenosis in mammals, wherein the compound administered has the Formula XII and wherein W^{XII} is of the formula W^{XII}1.17.

A method for preventing or treating atherosclerosis or restenosis in mammals, wherein the compound administered has the Formula XII and wherein W^{XII} is of the formula W^{XII}1.18.

A method for preventing or treating atherosclerosis or restenosis in mammals, wherein the compound administered has the Formula XII and wherein W^{XII} is of the formula W^{XII}1.19.

A method for preventing or treating atherosclerosis or restenosis in mammals, wherein the compound administered has the Formula XII and wherein W^{XII} is of the formula W^{XII}1.20.

A method for preventing or treating atherosclerosis or restenosis in mammals, wherein the compound administered has the Formula XII and wherein W^{XII} is of the formula W^{XII}1.21.

A method for preventing or treating atherosclerosis or restenosis in mammals, wherein the compound administered has the Formula XII and wherein W^{XII} is of the formula W^{XII}1.22.

A method for preventing or treating atherosclerosis or restenosis in mammals, wherein the compound administered has the Formula XII and wherein W^{XII} is of the formula W^{XII}1.23.

A method for preventing or treating atherosclerosis or restenosis in mammals, wherein the compound administered has the Formula XII and wherein W^{XII} is of the formula W^{XII}3.

A method for preventing or treating atherosclerosis or restenosis in mammals, wherein the compound administered has the Formula XII and wherein W^{XII} is of the formula W^{XII}3.1.

A method for preventing or treating atherosclerosis or restenosis in mammals, wherein the compound administered has the Formula XII and wherein W^{XII} is of the formula W^{XII}3.2.

A method for preventing or treating atherosclerosis or restenosis in mammals, wherein the compound administered has the Formula XII and wherein W^{XII} is of the formula W^{XII}3.3.

A method for preventing or treating atherosclerosis or restenosis in mammals, wherein the compound administered has the Formula XII and wherein W^{XII} is of the formula W^{XII}3.4.

A method for preventing or treating atherosclerosis or restenosis in mammals, wherein the compound administered has the Formula XII and wherein W^{XII} is of the formula W^{XII}3.5.

A method for preventing or treating atherosclerosis or restenosis in mammals, wherein the compound administered has the Formula XII and wherein W^{XII} is of the formula W^{XII}3.6.

A method for preventing or treating atherosclerosis or restenosis in mammals, wherein the compound administered has the Formula XII and wherein W^{XII} is of the formula W^{XII}3.7.

A method for preventing or treating atherosclerosis or restenosis in mammals, wherein the compound administered has the Formula XII and wherein W^{XII} is of the formula W^{XII}3.8.

A method for preventing or treating atherosclerosis or restenosis in mammals, wherein the compound administered has the Formula XII and wherein W^{XII} is of the formula W^{XII}3.9.

A method for preventing or treating atherosclerosis or restenosis in mammals, wherein the compound administered has the Formula XII and wherein W^{XII} is of the formula W^{XII}3.10.

A method for preventing or treating atherosclerosis or restenosis in mammals, wherein the compound administered has the Formula XII and wherein W^{XII} is of the formula W^{XII}3.11.

A method for preventing or treating atherosclerosis or restenosis in mammals, wherein the compound administered has the Formula XII and wherein W^{XII} is of the formula W^{XII}3.12.

A method for preventing or treating atherosclerosis or restenosis in mammals, wherein the compound administered has the Formula XII and wherein WXII is of the formula W^{XII}3.13.

A method for preventing or treating atherosclerosis or restenosis in mammals, wherein the compound administered has the Formula XII and wherein W^{XII} is of the formula W^{XII}3.14.

A method for preventing or treating atherosclerosis or restenosis in mammals, wherein the compound administered has the Formula XII and wherein W^{XII} is of the formula W^{XII}4.

Specific examples of W^{XII}1 include,

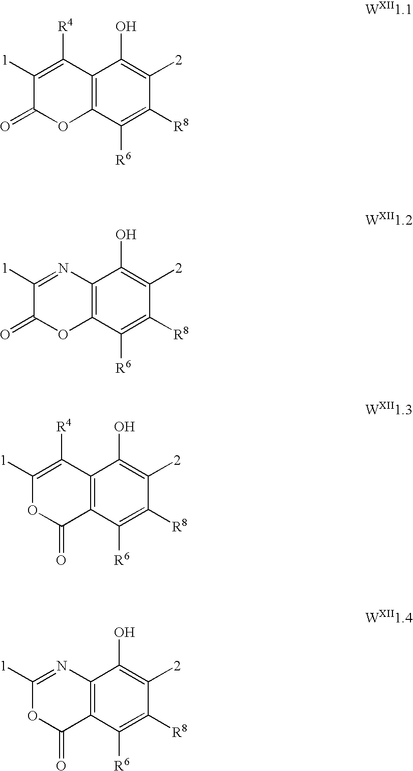

Specific examples of W^{XII}3 include,

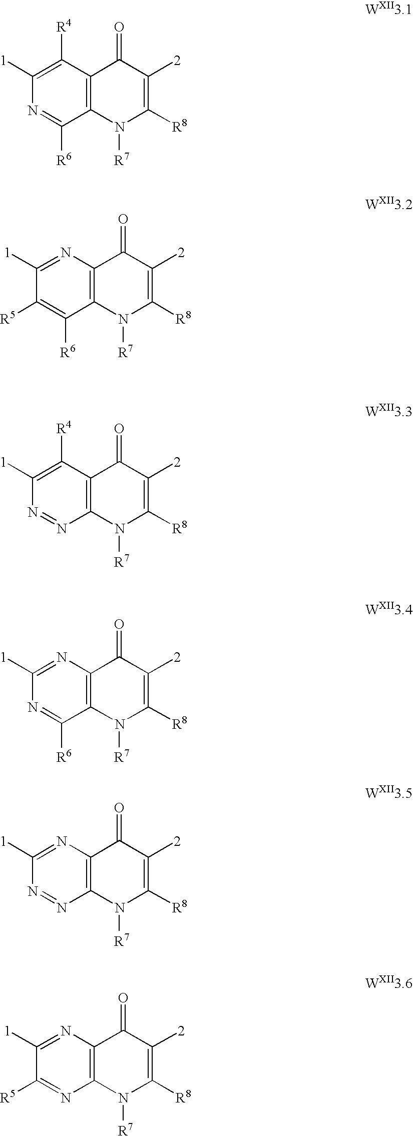

Specific examples of W^{XII}4 include,

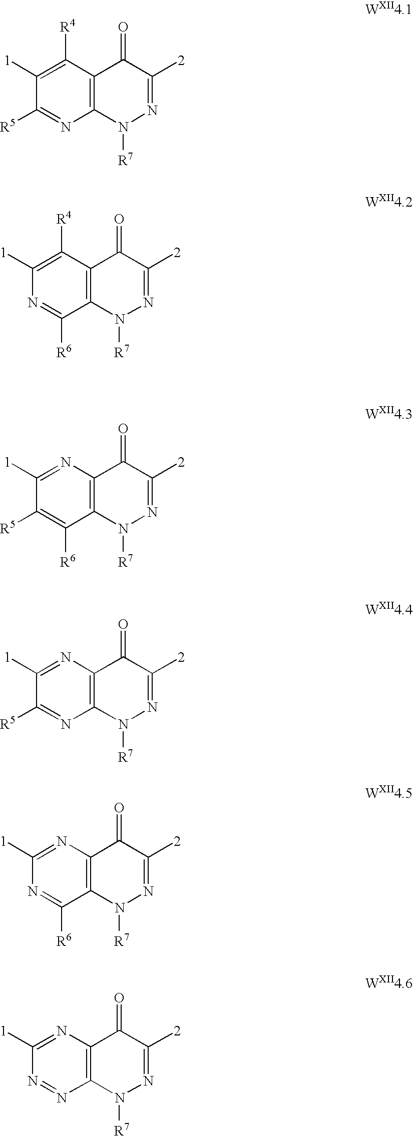

Specific examples of W^{XII}6 include,

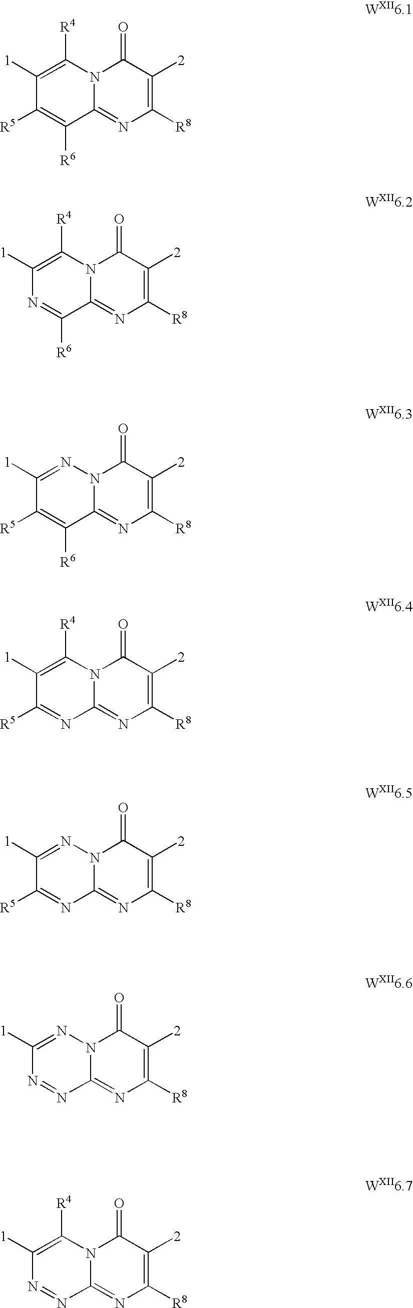

Specific examples of W^{XII}7 include,

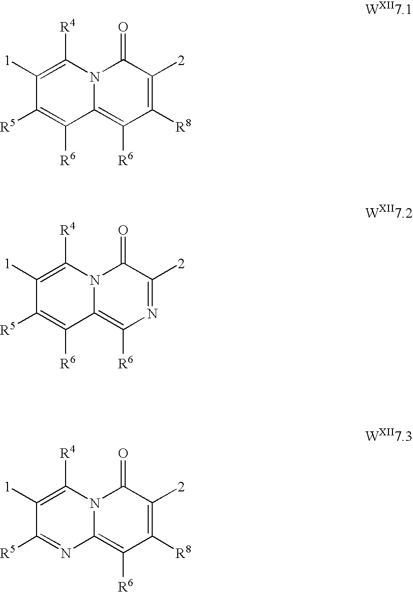

Specific examples of W^{XII}8 include,

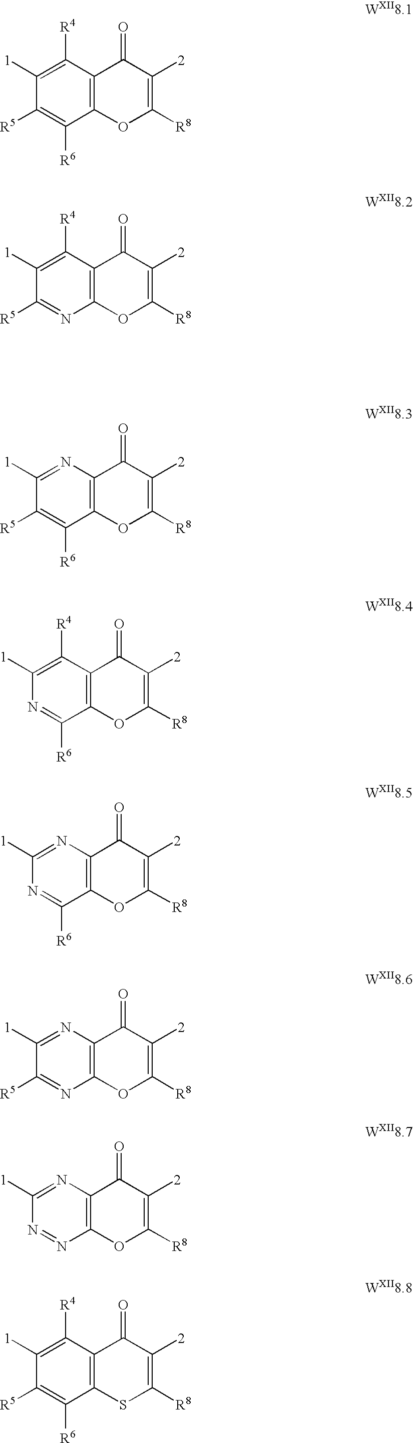

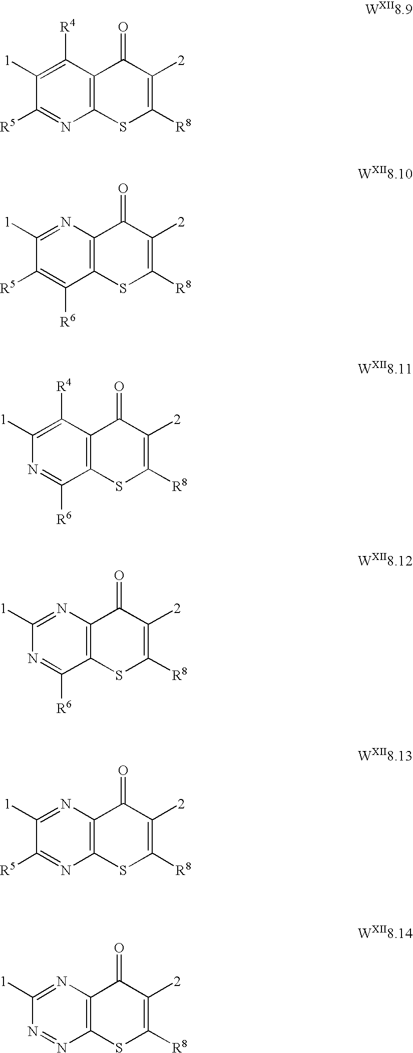

Specific examples of W^{XII}9 include,

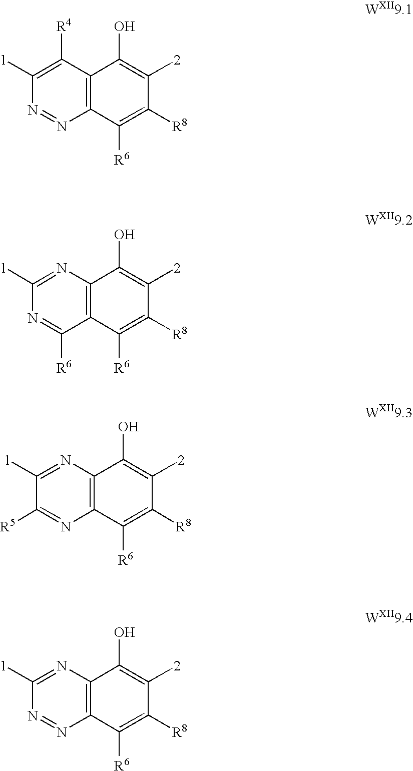

Specific examples of W^{XII}10 include,

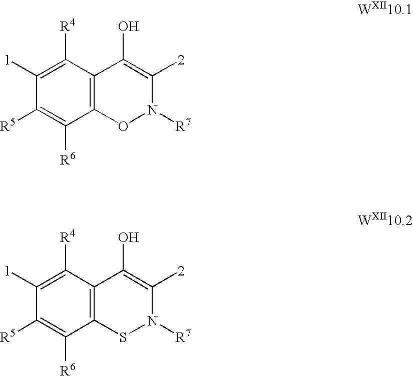

Specific examples of W^{XII}11 include,

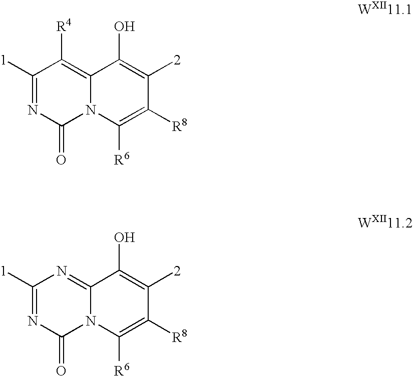

Specific examples of W^{XII}13 include,

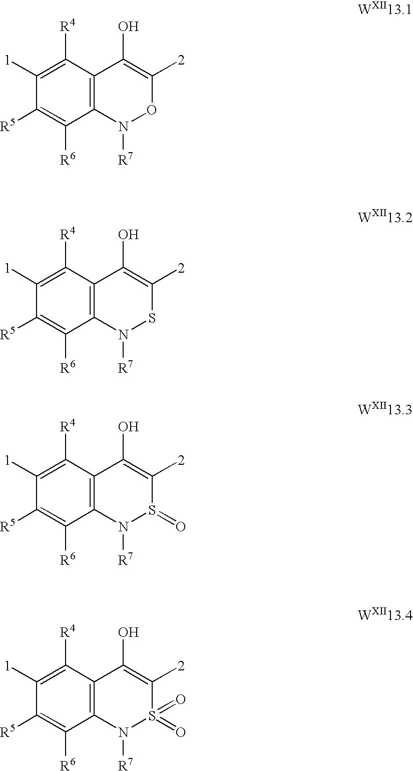

Specific examples of W^{XII}14 include,

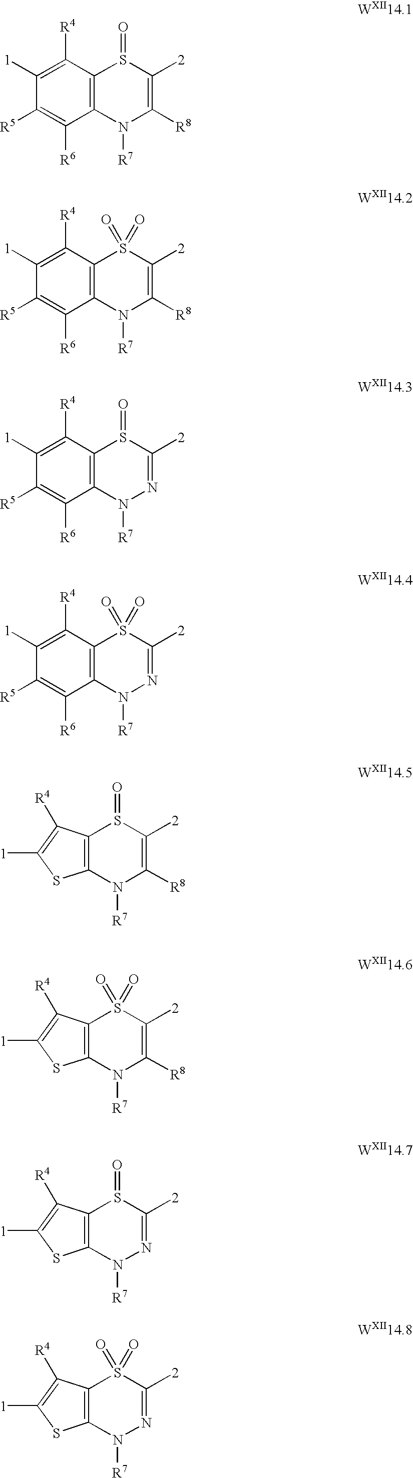

Specific examples of W^{XII}16 include,

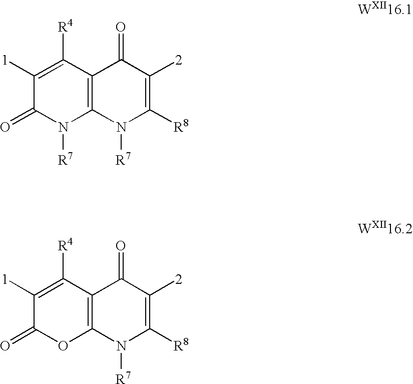

Specific examples of W^{XII}19 include,

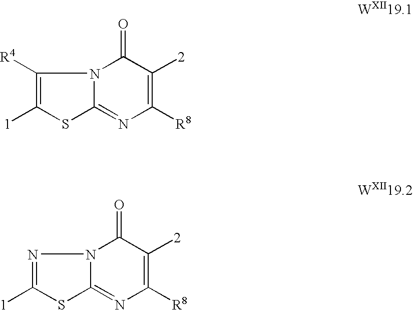

Specific examples of W^{XII}20 include,

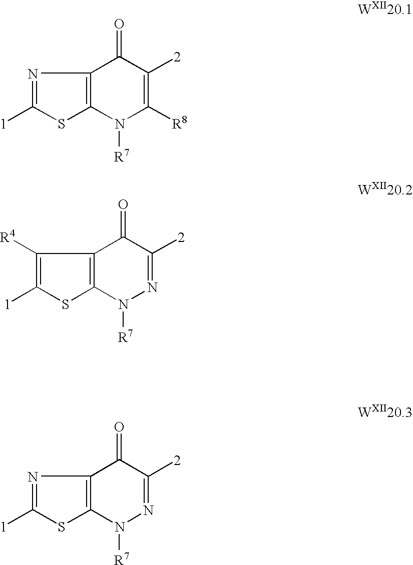

Specific examples of W^{XII}21 include,

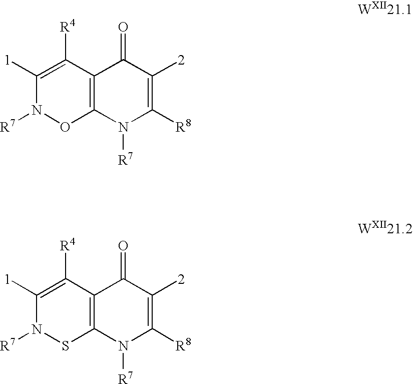

Specific examples of W^{XII}22 include,

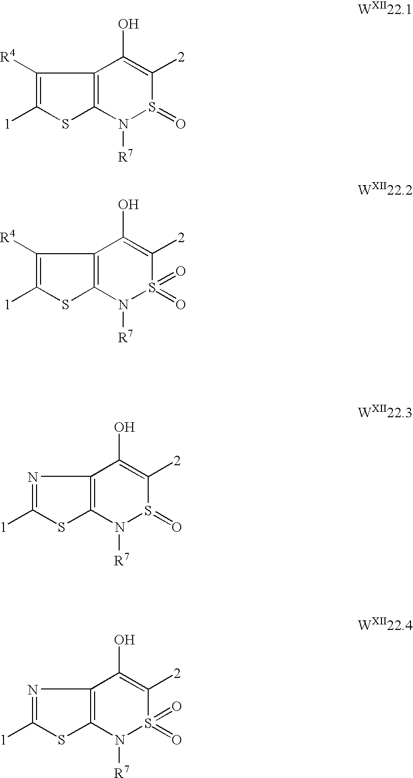

Particularly preferred compounds are those where X is Cl and G is 4-morpholinylmethyl.

A method for preventing or treating atherosclerosis or restenosis in mammals, wherein the compound administered has the Formula XII and wherein X^{XII} is Cl.

A method for preventing or treating atherosclerosis or restenosis in mammals, wherein the compound administered has the Formula XII and wherein G^{XII} is 4-morpholinylmethyl.

A method for preventing or treating atherosclerosis or restenosis in mammals, wherein the compound administered has the Formula XII and wherein G^{XII} is 3-hydroxy-1-propynyl.

A method for preventing or treating atherosclerosis or restenosis in mammals, wherein the compound administered has the Formula XII and wherein G^{XII} is tetrahydro-2H-pyran-4-ylmethyl.

A method for preventing or treating atherosclerosis or restenosis in mammals, wherein the compound administered is selected from the group consisting of:

(1) N-(4-chlorobenzyl)-4-hydroxy-6-(4-morpholinylmethyl)-2-oxo-2H-pyrano[2,3-c]pyridine-3-carboxamide;

(2) N-(4-chlorobenzyl)-4-hydroxy-6-(3-hydroxy-1-propynyl)-1-oxo-1H-isochromene-3-carboxamide;

(3) N-(4-chlorobenzyl)-4-hydroxy-1-oxo-6-(tetrahydro-2H-pyran-4-ylmethyl)-1H-isochromene-3-carboxamide;

(4) N-(4-chlorobenzyl)-4-hydroxy-6-(4-morpholinylmethyl)-1-oxo-1H-isochromene-3-carboxamide;

(5) N-(4-chlorobenzyl)-5-hydroxy-3-(3-hydroxy-1-propynyl)-8-oxo-7,8-dihydro[1,7]naphthyridine-6-carboxamide;

(6) N-(4-chlorobenzyl)-4-hydroxy-6-(3-hydroxy-1-propynyl)-1-oxo-1,2-dihydro-3-isoquinolinecarboxamide;

(7) N-(4-chlorobenzyl)-4-hydroxy-6-(4-morpholinylmethyl)-1-oxo-1,2-dihydro-3-isoquinolinecarboxamide;

(8) N-(4-chlorobenzyl)-4-hydroxy-6-(3-hydroxy-1-propynyl)[1,7]naphthyridine-3-carboxamide;

(9) N-(4-chlorobenzyl)-8-ethoxy-4-hydroxy-6-(3-hydroxy-1-propynyl)[1,7]naphthyridine-3-carboxamide;

(10) N-(4-chlorobenzyl)-4-hydroxy-6-(4-morpholinylmethyl)[1,7]naphthyridine-3-carboxamide;

(11) N-(4-chlorobenzyl)-8-ethoxy-4-hydroxy-6-(4-morpholinylmethyl)[1,7]naphthyridine-3-carboxamide;

(12) N-(4-chlorobenzyl)-4-hydroxy-6-(3-hydroxy-1-propynyl)[1,5]naphthyridine-3-carboxamide;

(13) N-(4-chlorobenzyl)-4-hydroxy-6-(4-morpholinylmethyl)[1,5]naphthyridine-3-carboxamide;

(14) N-(4-chlorobenzyl)-4-hydroxy-6-(tetrahydro-2H-pyran-4-ylmethyl)[1,5]naphthyridine-3-carboxamide;

(15) N-(4-chlorobenzyl)-8-hydroxy-2-(3-hydroxy-1-propynyl)pyrido[3,2-d]pyrimidine-7-carboxamide;

(16) N-(4-chlorobenzyl)-8-hydroxy-2-(4-morpholinylmethyl)pyrido[3,2-d]pyrimidine-7-carboxamide;

(17) N-(4-chlorobenzyl)-5-hydroxy-3-(4-morpholinylmethyl)[1,7]naphthyridine-6-carboxamide;

(18) N-(4-chlorobenzyl)-5-hydroxy-3-(3-hydroxy-1-propynyl)[1,7]naphthyridine-6-carboxamide;

(19) N-(4-chlorobenzyl)-5-hydroxy-3-(tetrahydro-2H-pyran-4-ylmethyl)[1,7]naphthyridine-6-carboxamide;

(20) N-(4-chlorobenzyl)-4-hydroxy-6-(4-morpholinylmethyl)-3-isoquinolinecarboxamide;

(21) N-(4-chlorobenzyl)-4-hydroxy-6-(3-hydroxy-1-propynyl)-3-isoquinolinecarboxamide;

(22) N-(4-chlorobenzyl)-4-hydroxy-6-(tetrahydro-2H-pyran-4-ylmethyl)-3-isoquinolinecarboxamide; or a pharmaceutically acceptable salt thereof.

A method of preventing or treating atherosclerosis or restenosis in a mammal, wherein the compound administered has the Formula XII and wherein said mammal is a human.

A method of preventing or treating atherosclerosis or restenosis in a mammal, wherein the compound administered has the Formula XII and wherein said mammal is a food animal or companion animal.

A method of preventing or treating atherosclerosis or restenosis in a mammal, wherein the compound administered has the Formula XII and wherein the amount administered is from about 0.1 to about 300 mg/kg of body weight.

A method of preventing or treating atherosclerosis or restenosis in a mammal, wherein the compound administered has the Formula XII and wherein the amount administered is from about 1.0 to about 30 mg/kg of body weight.

A method of preventing or treating atherosclerosis or restenosis in a mammal, wherein the compound administered has the Formula XII and wherein the compound is administered parenterally, topically, intravaginally, orally, or rectally.

A method of preventing or treating atherosclerosis or restenosis in a mammal, wherein the compound administered has the Formula XIII and wherein W^{XIII} is of the formula W2.1, as set forth in the specification of application Ser. No. 09/887,226 filed Jun. 22, 2001.

A method of preventing or treating atherosclerosis or restenosis in a mammal, wherein the compound administered has the Formula XIII and wherein W^{XIII} is of the formula W2.2, as set forth in the specification of application Ser. No. 09/887,226, filed Jun. 22, 2001.

A method of preventing or treating atherosclerosis or restenosis in a mammal, wherein the compound administered has the Formula XIII and wherein X^{XIII} is Cl.

A method of preventing or treating atherosclerosis or restenosis in a mammal, wherein the compound administered has the Formula XIII and wherein G^{XIII} is 4-morpholinylmethyl.

A method of preventing or treating atherosclerosis or restenosis in a mammal, wherein the compound administered has the Formula XIII and wherein G^{XIII} is 3-hydroxy-1-propynyl.

A method of preventing or treating atherosclerosis or restenosis in a mammal, wherein the compound administered has the Formula XIII and wherein G^{XIII} is tetrahydro-2H-pyran-4-ylmethyl.

A method of preventing or treating atherosclerosis or restenosis in a mammal, wherein the compound administered is selected from the group consisting of:

(1) N-(4-chlorobenzyl)-5-hydroxy-3-(4-morpholinylmethyl)-6-isoquinolinecarboxamide;

(2) N-(4-chlorobenzyl)-5-hydroxy-3-(tetrahydro-2H-pyran-4-ylmethyl)-6-isoquinolinecarboxamide;

(3) N-(4-chlorobenzyl)-5-hydroxy-3-(3-hydroxy-1-propynyl)-6-isoquinolinecarboxamide;

(4) N-(4-chlorobenzyl)-5-hydroxy-3-(4-morpholinylmethyl)-6-quinolinecarboxamide; or a pharmaceutically acceptable salt thereof.

A use of a compound of Formula XIII to prepare a medicament for treating or preventing atherosclerosis or restenosis in a mammal.

A use of a compound of Formula XIII to prepare a medicament for treating or preventing atherosclerosis or restenosis in a mammal, wherein said mammal is a human.

A use of a compound of Formula XIII to prepare a medicament for treating or preventing atherosclerosis or restenosis in a mammal, wherein said mammal is a food animal or companion animal.

A use of a compound of Formula XIII to prepare a medicament for treating or preventing atherosclerosis or restenosis in a mammal, wherein the amount administered is from about 0.1 to about 300 mg/kg of body weight.

A use of a compound of Formula XIII to prepare a medicament for treating or preventing atherosclerosis or restenosis in a mammal, wherein the amount administered is from about 1.0 to about 30 mg/kg of body weight.

A use of a compound of Formula XIII to prepare a medicament for treating or preventing atherosclerosis or restenosis in a mammal, wherein the compound is administered parenterally, topically, intravaginally, orally, or rectally.

A method for preventing or treating atherosclerosis or restenosis, wherein the compound administered has the Formula XIV and wherein W^{XIV} is of the formula W1^{XIV}.

A method for preventing or treating atherosclerosis or restenosis, wherein the compound administered has the Formula XIV and wherein W^{XIV} is of the formula W1.1^{XIV}.

A method for preventing or treating atherosclerosis or restenosis, wherein the compound administered has the Formula XIV and wherein W^{XIV} is of the formula W2^{XIV}.

A method for preventing or treating atherosclerosis or restenosis, wherein the compound administered has the Formula XIV and wherein W^{XIV} is of the formula W3^{XIV}.

A method for preventing or treating atherosclerosis or restenosis, wherein the compound administered has the Formula XIV and wherein W^{XIV} is of the formula W3.1^{XIV}.

A method for preventing or treating atherosclerosis or restenosis, wherein the compound administered has the Formula XIV and wherein W^{XIV} is of the formula W3.2^{XIV}.

A method for preventing or treating atherosclerosis or restenosis, wherein the compound administered has the Formula XIV and wherein W^{XIV} is of the formula W3.3^{XIV}.

A method for preventing or treating atherosclerosis or restenosis, wherein the compound administered has the Formula XIV and wherein W^{XIV} is of the formula W3.4^{XIV}.

A method for preventing or treating atherosclerosis or restenosis, wherein the compound administered has the Formula XIV and wherein W^{XIV} is of the formula W4^{XIV}.

A method for preventing or treating atherosclerosis or restenosis, wherein the compound administered has the Formula XIV and wherein W^{XIV} is of the formula W4.1^{XIV}.

A method for preventing or treating atherosclerosis or restenosis, wherein the compound administered has the Formula XIV and wherein W^{XIV} is of the formula W4.2^{XIV}.

A method for preventing or treating atherosclerosis or restenosis, wherein the compound administered has the Formula XIV and wherein W^{XIV} is of the formula W5^{XIV}.

A method for preventing or treating atherosclerosis or restenosis, wherein the compound administered has the Formula XIV and wherein W^{XIV} is of the formula W6^{XIV}.

A method for preventing or treating atherosclerosis or restenosis, wherein the compound administered has the Formula XIV and wherein W^{XIV} is of the formula W6.1^{XIV}.

A method for preventing or treating atherosclerosis or restenosis, wherein the compound administered has the Formula XIV and wherein W^{XIV} is of the formula W6.2^{XIV}.

A method for preventing or treating atherosclerosis or restenosis, wherein the compound administered has the Formula XIV and wherein W^{XIV} is of the formula W6.3^{XIV}.

A method for preventing or treating atherosclerosis or restenosis, wherein the compound administered has the Formula XIV and wherein W^{XIV} is of the formula W6.4^{XIV}.

A method for preventing or treating atherosclerosis or restenosis, wherein the compound administered has the Formula XIV and wherein W^{XIV} is of the formula W6.5^{XIV}.

A method for preventing or treating atherosclerosis or restenosis, wherein the compound administered has the Formula XIV and wherein W^{XIV} is of the formula W7^{XIV}.

A method for preventing or treating atherosclerosis or restenosis, wherein the compound administered has the Formula XIV and wherein W^{XIV} is of the formula W7.1^{XIV}.

A method for preventing or treating atherosclerosis or restenosis, wherein the compound administered has the Formula XIV and wherein W^{XIV} is of the formula W7.2^{XIV}.

A method for preventing or treating atherosclerosis or restenosis, wherein the compound administered has the Formula XIV and wherein W^{XIV} is of the formula W7.3^{XIV}.

A method for preventing or treating atherosclerosis or restenosis, wherein the compound administered has the Formula XIV and wherein W^{XIV} is of the formula W8^{XIV}.

A method for preventing or treating atherosclerosis or restenosis, wherein the compound administered has the Formula XIV and wherein W^{XIV} is of the formula W8.1^{XIV}.

A method for preventing or treating atherosclerosis or restenosis, wherein the compound administered has the Formula XIV and wherein W^{XIV} is of the formula W8.2^{XIV}.

A method for preventing or treating atherosclerosis or restenosis, wherein the compound administered has the Formula XIV and wherein W^{XIV} is of the formula W8.8^{XIV}.

A method for preventing or treating atherosclerosis or restenosis, wherein the compound administered has the Formula XIV and wherein W^{XIV} is of the formula W8.9^{XIV}.

A method for preventing or treating atherosclerosis or restenosis, wherein the compound administered has the Formula XIV and wherein W^{XIV} is of the formula W9^{XIV}.

A method for preventing or treating atherosclerosis or restenosis, wherein the compound administered has the Formula XIV and wherein W^{XIV} is of the formula W10^{XIV}.

A method for preventing or treating atherosclerosis or restenosis, wherein the compound administered has the Formula XIV and wherein W^{XIV} is of the formula W10.1^{XIV}.

A method for preventing or treating atherosclerosis or restenosis, wherein the compound administered has the Formula XIV and wherein W^{XIV} is of the formula W10.2^{XIV}.

A method for preventing or treating atherosclerosis or restenosis, wherein the compound administered has the Formula XIV and wherein W^{XIV} is of the formula W11^{XIV}.

A method for preventing or treating atherosclerosis or restenosis, wherein the compound administered has the Formula XIV and wherein W^{XIV} is of the formula W12^{XIV}.

A method for preventing or treating atherosclerosis or restenosis, wherein the compound administered has the Formula XIV and wherein W^{XIV} is of the formula W13^{XIV}.

A method for preventing or treating atherosclerosis or restenosis, wherein the compound administered has the Formula XIV and wherein W^{XIV} is of the formula W13.4^{XIV}.

A method for preventing or treating atherosclerosis or restenosis, wherein the compound administered has the Formula XIV and wherein W^{XIV} is of the formula W14^{XIV}.

A method for preventing or treating atherosclerosis or restenosis, wherein the compound administered has the Formula XIV and wherein W^{XIV} is of the formula W14.1^{XIV}.

A method for preventing or treating atherosclerosis or restenosis, wherein the compound administered has the Formula XIV and wherein W^{XIV} is of the formula W14.8^{XIV}.

A method for preventing or treating atherosclerosis or restenosis, wherein the compound administered has the Formula XIV and wherein W^{XIV} is of the formula W15^{XIV}.

A method for preventing or treating atherosclerosis or restenosis, wherein the compound administered has the Formula XIV and wherein W^{XIV} is of the formula W16^{XIV}.

A method for preventing or treating atherosclerosis or restenosis, wherein the compound administered has the Formula XIV and wherein W^{XIV} is of the formula W16.1^{XIV}.

A method for preventing or treating atherosclerosis or restenosis, wherein the compound administered has the Formula XIV and wherein W^{XIV} is of the formula W17^{XIV}.

A method for preventing or treating atherosclerosis or restenosis, wherein the compound administered has the Formula XIV and wherein W^{XIV} is of the formula W18^{XIV}.

A method for preventing or treating atherosclerosis or restenosis, wherein the compound administered has the Formula XIV and wherein W^{XIV} is of the formula W19^{XIV}.

A method for preventing or treating atherosclerosis or restenosis, wherein the compound administered has the Formula XIV and wherein W^{XIV} is of the formula W19.1^{XIV}.

A method for preventing or treating atherosclerosis or restenosis, wherein the compound administered has the Formula XIV and wherein W^{XIV} is of the formula W19.2^{XIV}.

A method for preventing or treating atherosclerosis or restenosis, wherein the compound administered has the Formula XIV and wherein W^{XIV} is of the formula W20^{XIV}.

A method for preventing or treating atherosclerosis or restenosis, wherein the compound administered has the Formula XIV and wherein W^{XIV} is of the formula W20.1^{XIV}.

A method for preventing or treating atherosclerosis or restenosis, wherein the compound administered has the Formula XIV and wherein W^{XIV} is of the formula W20.2^{XIV}.

A method for preventing or treating atherosclerosis or restenosis, wherein the compound administered has the Formula XIV and wherein W^{XIV} is of the formula W21^{XIV}.

A method for preventing or treating atherosclerosis or restenosis, wherein the compound administered has the Formula XIV and wherein W^{XIV} is of the formula W22^{XIV}.

A method for preventing or treating atherosclerosis or restenosis, wherein the compound administered has the Formula XIV and wherein W^{XIV} is of the formula W22.2^{XIV}.

A method for preventing or treating atherosclerosis or restenosis, wherein the compound administered has the Formula XIV and wherein X^{XIV} is Cl.

A method for preventing or treating atherosclerosis or restenosis, wherein the compound administered has the Formula XIV and wherein G^{XIV} is 4-morpholinylmethyl.

A method for preventing or treating atherosclerosis or restenosis, wherein the compound administered has the Formula XIV and wherein G^{XIV} is 3-hydroxy-1-propynyl.

A method for preventing or treating atherosclerosis or restenosis, wherein the compound administered has the Formula XIV and wherein G^{XIV} is tetrahydro-2H-pyran-4-ylmethyl.

A method for preventing or treating atherosclerosis or restenosis, wherein the compound administered has the Formula XIV and wherein G^{XIV} is 3-hydroxypropyl.

A method for preventing or treating atherosclerosis or restenosis in a mammal, wherein the compound administered has the Formula XIV and is selected from the group consisting of:

(1) N-(4-chlorobenzyl)-5-hydroxy-3-(3-hydroxy-1-propynyl)-2-oxo-2H-chromene-6-carboxamide;

(2) N-(4-chlorobenzyl)-5-hydroxy-3-(3-hydroxypropyl)-2-oxo-2H-chromene-6-carboxamide;

(3) N-(4-chlorobenzyl)-5-hydroxy-3-(4-morpholinylmethyl)-2-oxo-2H-chromene-6-carboxamide;

(4) N-(4-chlorobenzyl)-5-hydroxy-4-methyl-3,8-bis(4-morpholinylmethyl)-2-oxo-2H-chromene-6-carboxamide;

(5) N-(4-chlorobenzyl)-5-hydroxy-3-(3-hydroxy-1-propynyl)-1-methyl-2-oxo-1,2-dihydro-6-quinolinecarboxamide;

(6) N-(4-chlorobenzyl)-5-hydroxy-3-(3-hydroxypropyl)-1-methyl-2-oxo-1,2-dihydro-6-quinolinecarboxamide;

(7) N-(4-chlorobenzyl)-5-hydroxy-1-methyl-3-(4-morpholinylmethyl)-2-oxo-1,2-dihydro-6-quinolinecarboxamide;

(8) N-(4-chlorobenzyl)-6-(3-hydroxy-1-propynyl)-1-methyl-4-oxo-1,4-dihydro[1,7]naphthyridine-3-carboxamide;

(9) N-(4-chlorobenzyl)-6-(3-hydroxypropyl)-1-methyl-4-oxo-1,4-dihydro[1,7]naphthyridine-3-carboxamide;

(10) N-(4-chlorobenzyl)-1-methyl-6,8-bis(morpholin-4-ylmethyl)-4-oxo-1,4-dihydro-1,7-naphthyridine-3-carboxamide;

(11) N-(4-chlorobenzyl)-8-ethoxy-6-(3-hydroxy-1-propynyl)-1-methyl-4-oxo-1,4-dihydro[1,7]naphthyridine-3-carboxamide;

(12) N-(4-chlorobenzyl)-8-ethoxy-6-(3-hydroxypropyl)-1-methyl-4-oxo-1,4-dihydro[1,7]naphthyridine-3-carboxamide;

(13) N-(4-chlorobenzyl)-1-methyl-6-(4-morpholinylmethyl)-4-oxo-1,4-dihydro[1,7]naphthyridine-3-carboxamide;

(14) 8-chloro-N-(4-chlorobenzyl)-1-methyl-6-(4-morpholinylmethyl)-4-oxo-1,4-dihydro1,7]naphthyridine-3-carboxamide;

(15) N-(4-chlorobenzyl)-8-ethoxy-1-methyl-6-(4-morpholinylmethyl)-4-oxo-1,4-dihydro[1,7]naphthyridine-3-carboxamide;

(16) N-(4-chlorobenzyl)-6-(3-hydroxypropyl)-1-methyl-4-oxo-1,4-dihydro[1,5]naphthyridine-3-carboxamide;

(17) N-(4-chlorobenzyl)-6-(3-hydroxy-1-propynyl)-1-methyl-4-oxo-1,4-dihydro[1,5]naphthyridine-3-carboxamide;

(18) N-(4-chlorobenzyl)-1-methyl-6-(4-morpholinylmethyl)-4-oxo-1,4-dihydro[1,5]naphthyridine-3-carboxamide;

(19) N-(4-chlorobenzyl)-1-methyl-4-oxo-6-(tetrahydro-2H-pyran-4-ylmethyl)-1,4-dihydro[1,5]naphthyridine-3-carboxamide;

(20) N-(4-chlorobenzyl)-8-ethyl-3-(4-morpholinylmethyl)-5-oxo-5,8-dihydropyrido[2,3-c]pyridazine-6-carboxamide;

(21) N-(4-chlorobenzyl)-2-(3-hydroxypropyl)-5-methyl-8-oxo-5,8-dihydropyrido[3,2-d]pyrimidine-7-carboxamide;

(22) N-(4-chlorobenzyl)-2-(3-hydroxy-1-propynyl)-5-methyl-8-oxo-5,8-dihydropyrido[3,2-d]pyrimidine-7-carboxamide;

(23) N-(4-chlorobenzyl)-5-methyl-2-(4-morpholinylmethyl)-8-oxo-5,8-dihydropyrido[3,2-d]pyrimidine-7-carboxamide;

(24) N-(4-chlorobenzyl)-6-(3-hydroxy-1-propynyl)-1-methyl-4-oxo-1,4-dihydropyrido[2,3-c]pyridazine-3-carboxamide;

(25) N-(4-chlorobenzyl)-6-(3-hydroxypropyl)-1-methyl-4-oxo-1,4-dihydropyrido[2,3-c]pyridazine-3-carboxamide;

(26) N-(4-chlorobenzyl)-1-methyl-6-(4-morpholinylmethyl)-4-oxo-1,4-dihydropyrido[2,3-c]pyridazine-3-carboxamide;

(27) N-(4-chlorobenzyl)-1-methyl-4-oxo-6-(tetrahydro-2H-pyran-4-ylmethyl)-1,4-dihydropyrido[2,3-c]pyridazine-3-carboxamide;

(28) N-(4-chlorobenzyl)-6-(3-hydroxypropyl)-4-oxo-1,4-dihydropyrido[3,4-c]pyridazine-3-carboxamide;

(29) N-(4-chlorobenzyl)-6-(3-hydroxy-1-propynyl)-4-oxo-1,4-dihydropyrido[3,4-c]pyridazine-3-carboxamide;

(30) N-(4-chlorobenzyl)-6-(3-hydroxypropyl)-1-methyl-4-oxo-1,4-dihydropyrido[3,4-c]pyridazine-3-carboxamide;

(31) N-(4-chlorobenzyl)-6-(3-hydroxy-1-propynyl)-1-methyl-4-oxo-1,4-dihydropyrido[3,4-c]pyridazine-3-carboxamide;

(32) N-(4-chlorobenzyl)-1-methyl-6-(4-morpholinylmethyl)-4-oxo-1,4-dihydropyrido[3,4-c]pyridazine-3-carboxamide;

(33) N-(4-chlorobenzyl)-6-(4-morpholinylmethyl)-4-oxo-1,4-dihydropyrido[3,4-c]pyridazine-3-carboxamide;

(34) N-(4-chlorobenzyl)-7-(3-hydroxy-1-propynyl)-4-oxo-4H-pyrido[1,2-a]pyrimidine-3-carboxamide;

(35) N-(4-chlorobenzyl)-7-(3-hydroxypropyl)-4-oxo-4H-pyrido[1,2-a]pyrimidine-3-carboxamide;

(36) N-(4-chlorobenzyl)-7-(4-morpholinylmethyl)-4-oxo-4H-pyrido[1,2-a]pyrimidine-3-carboxamide;

(37) N-(4-chlorobenzyl)-4-hydroxy-7-(3-hydroxy-1-propynyl)-2-oxo-2H-pyrido[1,2-a]pyrimidine-3-carboxamide;

(38) N-(4-chlorobenzyl)-2-hydroxy-7-(3-hydroxypropyl)-4-oxo-4H-pyrido[1,2-a]pyrimidine-3-carboxamide;

(39) N-(4-chlorobenzyl)-7-(3-hydroxypropyl)-4-oxo-4H-pyrazino[1,2-a]pyrimidine-3-carboxamide;

(40) N-(4-chlorobenzyl)-7-(3-hydroxy-1-propynyl)-4-oxo-4H-pyrazino[1,2-a]pyrimidine-3-carboxamide;

(41) N-(4-chlorobenzyl)-7-(4-morpholinylmethyl)-4-oxo-4H-pyrazino[1,2-a]pyrimidine-3-carboxamide;

(42) N-(4-chlorobenzyl)-7-(3-hydroxy-1-propynyl)-4-oxo-4H-pyrimido[1,2-b]pyridazine-3-carboxamide;

(43) N-(4-chlorobenzyl)-7-(3-hydroxypropyl)-4-oxo-4H-pyrimido[1,2-b]pyridazine-3-carboxamide;

(44) N-(4-chlorobenzyl)-4-oxo-7-(tetrahydro-2H-pyran-4-ylmethyl)-4H-pyrimido[1,2-b]pyridazine-3-carboxamide;

(45) N-(4-chlorobenzyl)-7-(4-morpholinylmethyl)-4-oxo-4H-pyrimido[1,2-b]pyridazine-3-carboxamide;

(46) N-(4-chlorobenzyl)-7-(3-hydroxypropyl)-4-oxo-4H-pyrimido[1,2-a]pyrimidine-3-carboxamide;

(47) N-(4-chlorobenzyl)-7-(3-hydroxy-1-propynyl)-4-oxo-4H-pyrimido[1,2-a]pyrimidine-3-carboxamide;

(48) N-(4-chlorobenzyl)-4-oxo-7-(tetrahydro-2H-pyran-4-ylmethyl)-4H-pyrimido[1,2-a]pyrimidine-3-carboxamide;

(49) N-(4-chlorobenzyl)-7-(4-morpholinylmethyl)-4-oxo-4H-pyrimido[1,2-a]pyrimidine-3-carboxamide;

(50) N-(4-chlorobenzyl)-2-(3-hydroxypropyl)-8-oxo-8H-pyrimido[1,2-b][1,2,4]triazine-7-carboxamide;

(51) N-(4-chlorobenzyl)-2-(3-hydroxy-1-propynyl)-8-oxo-8H-pyrimido[1,2-b][1,2,4]triazine-7-carboxamide;

(52) N-(4-chlorobenzyl)-7-(4-morpholinylmethyl)-4-oxo-4H-quinolizine-3-carboxamide;

(53) N-(4-chlorobenzyl)-7-(3-hydroxypropyl)-4-oxo-4H-quinolizine-3-carboxamide;

(54) N-(4-chlorobenzyl)-7-(3-hydroxy-1-propynyl)-4-oxo-4H-quinolizine-3-carboxamide;

(55) N-(4-chlorobenzyl)-7-(3-hydroxy-1-propynyl)-4-oxo-4H-pyrido[1,2-a]pyrazine-3-carboxamide;

(56) N-(4-chlorobenzyl)-7-(3-hydroxypropyl)-4-oxo-4H-pyrido[1,2-a]pyrazine-3-carboxamide;

(57) N-(4-chlorobenzyl)-7-(4-morpholinylmethyl)-4-oxo-4H-pyrido[1,2-a]pyrazine-3-carboxamide;

(58) N-(4-chlorobenzyl)-3-(4-morpholinylmethyl)-6-oxo-6H-pyrido[1,2-a]pyrimidine-7-carboxamide;

(59) N-(4-chlorobenzyl)-6-(4-morpholinylmethyl)-4-oxo-4H-chromene-3-carboxamide;

(60) N-(4-chlorobenzyl)-6-(3-hydroxy-1-propynyl)-4-oxo-4H-chromene-3-carboxamide;

(61) N-(4-chlorobenzyl)-6-(((3R)-3-hydroxypyrrolidinyl)methyl)-4-oxo-4H-chromene-3-carboxamide;

(62) N-(4-chlorobenzyl)-6,8-bis(4-morpholinylmethyl)-4-oxo-4H-chromene-3-carboxamide;

(63) N-(4-chlorobenzyl)-6-(3-hydroxypropyl)-4-oxo-4H-chromene-3-carboxamide;

(64) N-(4-chlorobenzyl)-6-(3-hydroxypropyl)-4-oxo-4H-pyrano[2,3-b]pyridine-3-carboxamide;

(65) N-(4-chlorobenzyl)-6-(3-hydroxy-1-propynyl)-4-oxo-4H-pyrano[2,3-b]pyridine-3-carboxamide;

(66) N-(4-chlorobenzyl)-4-oxo-6-(tetrahydro-2H-pyran-4-ylmethyl)-4H-pyrano[2,3-b]pyridine-3-carboxamide;

(67) N-(4-chlorobenzyl)-6-(4-morpholinylmethyl)-4-oxo-4H-pyrano[2,3-b]pyridine-3-carboxamide;

(68) N-(4-chlorobenzyl)-6-(4-morpholinylmethyl)-4-oxo-4H-thiochromene-3-carboxamide;

(69) N-(4-chlorobenzyl)-6-(3-hydroxypropyl)-4-oxo-4H-thiopyrano[2,3-b]pyridine-3-carboxamide;

(70) N-(4-chlorobenzyl)-6-(3-hydroxy-1-propynyl)-4-oxo-4H-thiopyrano[2,3-b]pyridine-3-carboxamide;

(71) N-(4-chlorobenzyl)-4-oxo-6-(tetrahydro-2H-pyran-4-ylmethyl)-4H-thiopyrano[2,3-b]pyridine-3-carboxamide;

(72) N-(4-chlorobenzyl)-6-(4-morpholinylmethyl)-4-oxo-4H-thiopyrano[2,3-b]pyridine-3-carboxamide;

(73) N-(4-chlorobenzyl)-6-(3-hydroxy-1-propynyl)-4-oxo-4H-1,2-benzoxazine-3-carboxamide;

(74) N-(4-chlorobenzyl)-6-(3-hydroxypropyl)-4-oxo-4H-1,2-benzoxazine-3-carboxamide;

(75) N-(4-chlorobenzyl)-6-(4-morpholinylmethyl)-4-oxo-4H-1,2-benzoxazine-3-carboxamide;

(76) N-(4-chlorobenzyl)-4-oxo-6-(tetrahydro-2H-pyran-4-ylmethyl)-4H-1,2-benzoxazine-3-carboxamide;

(77) N-(4-chlorobenzyl)-6-(3-hydroxy-1-propynyl)-4-oxo-4H-1,2-benzthiazine-3-carboxamide;

(78) N-(4-chlorobenzyl)-6-(3-hydroxypropyl)-4-oxo-4H-1,2-benzthiazine-3-carboxamide;

(79) N-(4-chlorobenzyl)-6-(4-morpholinylmethyl)-4-oxo-4H-1,2-benzthiazine-3-carboxamide;

(80) N-(4-chlorobenzyl)-4-oxo-6-(tetrahydro-2H-pyran-4-ylmethyl)-4H-1,2-benzthiazine-3-carboxamide;

(81) N-(4-chlorobenzyl)-4-hydroxy-6-(3-hydroxy-1-propynyl)-1-methyl-1H-2,1-benzothiazine-3-carboxamide 2,2-dioxide;

(82) N-(4-chlorobenzyl)-4-hydroxy-1-methyl-6-(4-morpholinylmethyl)-1H-2,1-benzothiazine-3-carboxamide 2,2-dioxide;

(83) N-(4-chlorobenzyl)-4-methyl-7-(4-morpholinylmethyl)-4H-1,4-benzothiazine-2-carboxamide 1-oxide;

(84) N-(4-chlorobenzyl)-1-methyl-6-(4-morpholinylmethyl)-1H-4,1,2-benzothiadiazine-3-carboxamide 4,4-Dioxide;

(85) N-(4-chlorobenzyl)-1-methyl-6-(4-morpholinylmethyl)-1H-thieno[2,3-e][1,3,4]thiadiazine-3-carboxamide 4,4-dioxide;

(86) N-(4-chlorobenzyl)-6-(4-morpholinylmethyl)-1H-thieno[2,3-e][1,3,4]thiadiazine-3-carboxamide 4,4-dioxide;

(87) N-(4-chlorobenzyl)-6-(3-hydroxypropyl)-1H-thieno[2,3-e][1,3,4]thiadiazine-3-carboxamide 4,4-dioxide;

(88) N-(4-chlorobenzyl)-6-(3-hydroxy-1-propynyl)-1H-thieno[2,3-e][1,3,4]thiadiazine-3-carboxamide 4,4-dioxide;

(89) N-(4-chlorobenzyl)-6-(3-hydroxypropyl)-1-methyl-1H-thieno[2,3-e][1,3,4]thiadiazine-3-carboxamide 4,4-dioxide;

(90) N-(4-chlorobenzyl)-6-(3-hydroxy-1-propynyl)-1-methyl-1H-thieno[2,3-e][1,3,4]thiadiazine-3-carboxamide 4,4-dioxide;

(91) N-(4-chlorobenzyl)-6-(3-hydroxypropyl)-1,8-dimethyl-4,7-dioxo-1,4,7,8-tetrahydro[1,8]naphthyridine-3-carboxamide;

(92) N-(4-chlorobenzyl)-6-(3-hydroxy-1-propynyl)-1,8-dimethyl-4,7-dioxo-1,4,7,8-tetrahydro[1,8]naphthyridine-3-carboxamide;

(93) N-(4-chlorobenzyl)-1,8-dimethyl-6-(4-morpholinylmethyl)-4,7-dioxo-1,4,7,8-tetrahydro[1,8]naphthyridine-3-carboxamide;

(94) N-(4-chlorobenzyl)-7-(3-hydroxy-1-propynyl)-4-oxo-4H-pyrido[2,1-c][1,2,4]triazine-3-carboxamide;

(95) N-(4-chlorobenzyl)-7-(3-hydroxypropyl)-4-oxo-4H-pyrido[2,1-c][1,2,4]triazine-3-carboxamide;

(96) N-(4-chlorobenzyl)-7-(4-morpholinylmethyl)-4-oxo-4H-pyrido[2,1-c][1,2,4]triazine-3-carboxamide;

(97) N-(4-chlorobenzyl)-4-hydroxy-2-(4-morpholinylmethyl)-1-benzothiophene-5-carboxamide;

(98) N-(4-chlorobenzyl)-4-hydroxy-2-(3-hydroxypropyl)-1-benzothiophene-5-carboxamide;

(99) N-(4-chlorobenzyl)-2-(4-morpholinylmethyl)-5-oxo-5H-[1,3]thiazolo[3,2-a]pyrimidine-6-carboxamide;

(100) N-(4-chlorobenzyl)-5-hydroxy-2-(4-morpholinylmethyl)-7-oxo-7H-[1,3,4]thiadiazolo[3,2-a]pyrimidine-6-carboxamide;

(101) N-(4-chlorobenzyl)-4-methyl-2-(4-morpholinylmethyl)-7-oxo-4,7-dihydro[1,3]thiazolo[5,4-b]pyridine-6-carboxamide;

(102) N-(4-chlorobenzyl)-6-(3-hydroxy-1-propynyl)-1-methyl-4-oxo-1,4-dihydrothieno[2,3-c]pyridazine-3-carboxamide;

(103) N-(4-chlorobenzyl)-6-(3-hydroxypropyl)-1-methyl-4-oxo-1,4-dihydrothieno[2,3-c]pyridazine-3-carboxamide;

(104) N-(4-chlorobenzyl)-1-methyl-6-(4-morpholinylmethyl)-4-oxo-1,4-dihydrothieno[2,3-c]pyridazine-3-carboxamide;

(105) N-(4-chlorobenzyl)-6-(4-morpholinylmethyl)-4-oxo-1-phenyl-1,4-dihydrothieno[2,3-c]pyridazine-3-carboxamide;

(106) N-(4-chlorobenzyl)-1-methyl-4-oxo-6-(tetrahydro-2H-pyran-4-ylmethyl)-1,4-dihydrothieno[2,3-c]pyridazine-3-carboxamide;

(107) N-(4-chlorobenzyl)-4-hydroxy-6-(3-hydroxyprop-1-ynyl)-1-methyl-1H-thieno[2,3-c][1,2]thiazine-3-carboxamide 2,2-dioxide; or a pharmaceutically acceptable salt thereof.

A method for preventing or treating atherosclerosis or restenosis in a mammal, wherein the compound administered has the Formula XIV and is selected from the group consisting of:

(1) N-(4-chlorobenzyl)-7-(3-hydroxy-1-propynyl)-4-oxo-4H-pyrido[1,2-a]pyrimidine-3-carboxamide;

(2) N-(4-chlorobenzyl)-5-hydroxy-2-(4-morpholinylmethyl)-7-oxo-7H-[1,3,4]thiadiazolo[3,2-a]pyrimidine-6-carboxamide;

(3) N-(4-chlorobenzyl)-4-hydroxy-7-(3-hydroxy-1-propynyl)-2-oxo-2H-pyrido[1,2-a]pyrimidine-3-carboxamide;

(4) N-(4-chlorobenzyl)-2-(4-morpholinylmethyl)-5-oxo-5H-[1,3]thiazolo[3,2-a]pyrimidine-6-carboxamide;

(5) N-(4-chlorobenzyl)-4-hydroxy-2-(4-morpholinylmethyl)-1-benzothiophene-5-carboxamide;

(6) N-(4-chlorobenzyl)-6-(3-hydroxy-1-propynyl)-4-oxo-4H-chromene-3-carboxamide;

(7) N-(4-chlorobenzyl)-6-(3-hydroxy-1-propynyl)-4-oxo-4H-1,2-benzoxazine-3-carboxamide;

(8) N-(4-chlorobenzyl)-6-(3-hydroxy-1-propynyl)-1-methyl-4-oxo-1,4-dihydropyrido[2,3-c]pyridazine-3-carboxamide;

(9) N-(4-chlorobenzyl)-4-methyl-2-(4-morpholinylmethyl)-7-oxo-4,7-dihydro[1,3]thiazolo[5,4-b]pyridine-6-carboxamide;

(10) N-(4-chlorobenzyl)-4-hydroxy-6-(3-hydroxyprop-1-ynyl)-1-methyl-1H-thieno[2,3-c][1,2]thiazine-3-carboxamide 2,2-dioxide;

(11) N-(4-chlorobenzyl)-1-methyl-6-(4-morpholinylmethyl)-1H-4,1,2-benzothiadiazine-3-carboxamide 4,4-Dioxide;

(12) 8-chloro-N-(4-chlorobenzyl)-1-methyl-6-(4-morpholinylmethyl)-4-oxo-1,4-dihydro1,7]naphthyridine-3-carboxamide;

(13) N-(4-chlorobenzyl)-1-methyl-6-(4-morpholinylmethyl)-4-oxo-1,4-dihydro[1,7]naphthyridine-3-carboxamide;

(14) N-(4-chlorobenzyl)-1-methyl-6,8-bis(morpholin-4-ylmethyl)-4-oxo-1,4-dihydro-1,7-naphthyridine-3-carboxamide;

(15) N-(4-chlorobenzyl)-4-hydroxy-6-(3-hydroxy-1-propynyl)-1-methyl-1H-2,1-benzothiazine-3-carboxamide 2,2-dioxide;

(16) N-(4-chlorobenzyl)-6-(3-hydroxy-1-propynyl)-1-methyl-1H-thieno[2,3-e][1,3,4]thiadiazine-3-carboxamide 4,4-dioxide;

(17) N-(4-chlorobenzyl)-6-(3-hydroxy-1-propynyl)-1-methyl-4-oxo-1,4-dihydro[1,5]naphthyridine-3-carboxamide;

(18) N-(4-chlorobenzyl)-6-(4-morpholinylmethyl)-4-oxo-1-phenyl-1,4-dihydrothieno[2,3-c]pyridazine-3-carboxamide;

(19) N-(4-chlorobenzyl)-6-(4-morpholinylmethyl)-4-oxo-4H-chromene-3-carboxamide;

(20) N-(4-chlorobenzyl)-6,8-bis(4-morpholinylmethyl)-4-oxo-4H-chromene-3-carboxamide;

(21) N-(4-chlorobenzyl)-6-(((3R)-3-hydroxypyrrolidinyl)methyl)-4-oxo-4H-chromene-3-carboxamide; or a pharmaceutically acceptable salt thereof.

A method for preventing or treating atherosclerosis or restenosis in a mammal, wherein the compound administered has the Formula XIV and is selected from the group consisting of:

(1) N-(4-chlorobenzyl)-1-methyl-6-(4-morpholinylmethyl)-1H-4,1,2-benzothiadiazine-3-carboxamide 4,4-Dioxide;

(2) N-(4-chlorobenzyl)-4-hydroxy-6-(3-hydroxy-1-propynyl)-1-methyl-1H-2,1-benzothiazine-3-carboxamide 2,2-dioxide;

(3) N-(4-chlorobenzyl)-6-(3-hydroxy-1-propynyl)-1-methyl-1H-thieno[2,3-e][1,3,4]thiadiazine-3-carboxamide 4,4-dioxide;

(4) N-(4-chlorobenzyl)-6-(3-hydroxy-1-propynyl)-1-methyl-4-oxo-1,4-dihydro[1,5]naphthyridine-3-carboxamide; or or a pharmaceutically acceptable salt thereof.

A method for preventing or treating atherosclerosis or restenosis in a mammal, wherein the compound administered has the Formula XIV and is selected from the group consisting of:

(1) N-(4-chlorobenzyl)-4-hydroxy-7-(3-hydroxy-1-propynyl)-2-oxo-2H-pyrido[1,2-a]pyrimidine-3-carboxamide;

(2) N-(4-chlorobenzyl)-6-(3-hydroxy-1-propynyl)-4-oxo-4H-chromene-3-carboxamide;

(3) 8-chloro-N-(4-chlorobenzyl)-1-methyl-6-(4-morpholinylmethyl)-4-oxo-1,4-dihydro1,7]naphthyridine-3-carboxamide;

(4) N-(4-chlorobenzyl)-1-methyl-6,8-bis(morpholin-4-ylmethyl)-4-oxo-1,4-dihydro-1,7-naphthyridine-3-carboxamide;

(5) N-(4-chlorobenzyl)-6-(4-morpholinylmethyl)-4-oxo-4H-chromene-3-carboxamide;

(6) N-(4-chlorobenzyl)-6,8-bis(4-morpholinylmethyl)-4-oxo-4H-chromene-3-carboxamide;

(7) N-(4-chlorobenzyl)-6-(((3R)-3-hydroxypyrrolidinyl)methyl)-4-oxo-4H-chromene-3-carboxamide; or a pharmaceutically acceptable salt thereof.

A use of a compound of Formula XIV to prepare a medicament for treating or preventing atherosclerosis or restenosis in a mammal.

A use of a compound of Formula XIV to prepare a medicament for treating or preventing atherosclerosis or restenosis in a mammal, wherein said mammal is a human.

A use of a compound of Formula XIV to prepare a medicament for treating or preventing atherosclerosis or restenosis in a mammal, wherein said mammal is a livestock or companion animal.

A use of a compound of Formula XIV to prepare a medicament for treating or preventing atherosclerosis or restenosis in a mammal, wherein the amount administered is from about 0.1 to about 300 mg/kg of body weight.

A use of a compound of Formula XIV to prepare a medicament for treating or preventing atherosclerosis or restenosis in a mammal, wherein the amount administered is from about 1.0 to about 300 mg/kg of body weight.

A use of a compound of Formula XIV to prepare a medicament for treating or preventing atherosclerosis or restenosis in a mammal, wherein the compound is administered parenterally, topically, intravaginally, orally, or rectally.

A method of preventing or treating atherosclerosis or restenosis in a mammal, wherein the compound administered has the Formula XV and wherein W^{XV} is of the formula W1^{XV}.

A method of preventing or treating atherosclerosis or restenosis in a mammal, wherein the compound administered has the Formula XV and wherein W^{XV} is of the formula W1.1^{XV}.

A method of preventing or treating atherosclerosis or restenosis in a mammal, wherein the compound administered has the Formula XV and wherein W^{XV} is of the formula W1.3^{XV}.

A method of preventing or treating atherosclerosis or restenosis in a mammal, wherein the compound administered has the Formula XV and wherein W^{XV} is of the formula W1.5^{XV}.

A method of preventing or treating atherosclerosis or restenosis in a mammal, wherein the compound administered has the Formula XV and wherein W^{XV} is of the formula W1.6^{XV}.

A method of preventing or treating atherosclerosis or restenosis in a mammal, wherein the compound administered has the Formula XV and wherein W^{XV} is of the formula W1.7^{XV}.

A method of preventing or treating atherosclerosis or restenosis in a mammal, wherein the compound administered has the Formula XV and wherein W^{XV} is of the formula W1.8^{XV}.

A method of preventing or treating atherosclerosis or restenosis in a mammal, wherein the compound administered has the Formula XV and wherein W^{XV} is of the formula W1.9^{XV}.

A method of preventing or treating atherosclerosis or restenosis in a mammal, wherein the compound administered has the Formula XV and wherein W^{XV} is of the formula W1.10^{XV}.

A method of preventing or treating atherosclerosis or restenosis in a mammal, wherein the compound administered has the Formula XV and wherein W^{XV} is of the formula W1.11^{XV}.

A method of preventing or treating atherosclerosis or restenosis in a mammal, wherein the compound administered has the Formula XV and wherein W^{XV} is of the formula W1.14^{XV}.

A method of preventing or treating atherosclerosis or restenosis in a mammal, wherein the compound administered has the Formula XV and wherein W^{XV} is of the formula W1.15^{XV}.

A method of preventing or treating atherosclerosis or restenosis in a mammal, wherein the compound administered has the Formula XV and wherein W^{XV} is of the formula W1.17^{XV}.

A method of preventing or treating atherosclerosis or restenosis in a mammal, wherein the compound administered has the Formula XV and wherein W^{XV} is of the formula W1.18^{XV}.

A method of preventing or treating atherosclerosis or restenosis in a mammal, wherein the compound administered has the Formula XV and wherein W^{XV} is of the formula W1.19^{XV}.

A method of preventing or treating atherosclerosis or restenosis in a mammal, wherein the compound administered has the Formula XV and wherein W^{XV} is of the formula W1.20^{XV}.

A method of preventing or treating atherosclerosis or restenosis in a mammal, wherein the compound administered has the Formula XV and wherein W^{XV} is of the formula W1.23^{XV}.

A method of preventing or treating atherosclerosis or restenosis in a mammal, wherein the compound administered has the Formula XV and wherein W^{XV} is of the formula W1.24^{XV}.

A method of preventing or treating atherosclerosis or restenosis in a mammal, wherein the compound administered has the Formula XV and wherein W^{XV} is of the formula W1.25^{XV}.

A method of preventing or treating atherosclerosis or restenosis in a mammal, wherein the compound administered has the Formula XV and wherein W^{XV} is of the formula W1.26^{XV}.

A method of preventing or treating atherosclerosis or restenosis in a mammal, wherein the compound administered has the Formula XV and wherein W^{XV} is of the formula W1.52^{XV}.

A method of preventing or treating atherosclerosis or restenosis in a mammal, wherein the compound administered has the Formula XV and wherein W^{XV} is of the formula W2^{XV}.

A method of preventing or treating atherosclerosis or restenosis in a mammal, wherein the compound administered has the Formula XV and wherein W^{XV} is of the formula W3^{XV}.

A method of preventing or treating atherosclerosis or restenosis in a mammal, wherein the compound administered has the Formula XV and wherein W^{XV} is of the formula W3.1^{XV}.

A method of preventing or treating atherosclerosis or restenosis in a mammal, wherein the compound administered has the Formula XV and wherein W^{XV} is of the formula W4^{XV}.

A method of preventing or treating atherosclerosis or restenosis in a mammal, wherein the compound administered has the Formula XV and wherein W^{XV} is of the formula W4.1^{XV}.

A method of preventing or treating atherosclerosis or restenosis in a mammal, wherein the compound administered has the Formula XV and wherein W^{XV} is of the formula W5^{XV}.

A method of preventing or treating atherosclerosis or restenosis in a mammal, wherein the compound administered has the Formula XV and wherein W^{XV} is of the formula W6^{XV}.

A method of preventing or treating atherosclerosis or restenosis in a mammal, wherein the compound administered has the Formula XV and wherein W^{XV} is of the formula W7^{XV}.

A method of preventing or treating atherosclerosis or restenosis in a mammal, wherein the compound administered has the Formula XV and wherein W^{XV} is of the formula W8^{XV}.

A method of preventing or treating atherosclerosis or restenosis in a mammal, wherein the compound administered has the Formula XV and wherein X^{XV} is Cl.

A method of preventing or treating atherosclerosis or restenosis in a mammal, wherein the compound administered has the Formula XV and wherein G^{XV} is 4-morpholinylmethyl.

A method of preventing or treating atherosclerosis or restenosis in a mammal, wherein the compound administered has the Formula XV and wherein G^{XV} is 3-hydroxypropyl.

A method of preventing or treating atherosclerosis or restenosis in a mammal, wherein the compound administered has the Formula XV and wherein G^{XV} is 3-hydroxy-1-propynyl.

A method of preventing or treating atherosclerosis or restenosis in a mammal, wherein the compound administered has the Formula XV and wherein G^{XV} is tetrahydro-2H-pyran-4-ylmethyl.

A method of preventing or treating atherosclerosis or restenosis in a mammal, wherein the compound administered has the Formula XV and is hydrogen.

A method of preventing or treating atherosclerosis or restenosis in a mammal, wherein the compound administered has the Formula XV and where A^{XV} is CR^{XV-4} and B is CR^{XV-5}.

A method of preventing or treating atherosclerosis or restenosis in a mammal, wherein the compound administered has the Formula XV and where R^{XV-4} is hydrogen and R^{XV-5} is hydrogen.

A method of preventing or treating atherosclerosis or restenosis in a mammal, wherein the compound administered has the Formula XV and is selected from the group consisting of:

(1) N-(4-chlorobenzyl)-8-(4-morpholinylmethyl)-6-oxo-6H-imidazo[4,5,1-ij]quinoline-5-carboxamide;

(2) N-(4-chlorobenzyl)-8-(3-hydroxy-1-propynyl)-6-oxo-6H-imidazo[4,5,1-ij]quinoline-5-carboxamide;

(3) N-(4-chlorobenzyl)-8-(3-hydroxypropyl)-6-oxo-6H-imidazo[4,5,1-ij]quinoline-5-carboxamide;

(4) 1-amino-N-(4-chlorobenzyl)-8-(3-hydroxy-1-propynyl)-6-oxo-1,2-dihydro-6H-pyrrolo[3,2,1-ij]quinoline-5-carboxamide;

(5) 1-amino-N-(4-chlorobenzyl)-8-(3-hydroxypropyl)-6-oxo-1,2-dihydro-6H-pyrrolo[3,2,1-ij]quinoline-5-carboxamide;

(6) 1-amino-N-(4-chlorobenzyl)-8-(4-morpholinylmethyl)-6-oxo-1,2-dihydro-6H-pyrrolo[3,2,1-ij]quinoline-5-carboxamide;

(7) N-(4-chlorobenzyl)-2-(hydroxymethyl)-8-(3-hydroxy-1-propynyl)-6-oxo-1,2-dihydro-6H-pyrrolo[3,2,1-ij]quinoline-5-carboxamide;

(8) N-(4-chlorobenzyl)-8-(3-hydroxyprop-1-ynyl)-2,2-dimethyl-6-oxo-1,2-dihydro-6H-pyrrolo[3,2,1-ij]quinoline-5-carboxamide;

(9) 3-benzyl-N-(4-chlorobenzyl)-9-(3-hydroxy-1-propynyl)-7-oxo-2,3-dihydro-7H-[1,3,4]oxadiazino[6,5,4-ij]quinoline-6-carboxamide;

(10) 3-benzyl-N-(4-chlorobenzyl)-9-(3-hydroxypropyl)-7-oxo-2,3-dihydro-7H-[1,3,4]oxadiazino[6,5,4-ij]quinoline-6-carboxamide;

(11) 3-benzyl-N-(4-chlorobenzyl)-9-(4-morpholinylmethyl)-7-oxo-2,3-dihydro-7H-[1,3,4]oxadiazino[6,5,4-ij]quinoline-6-carboxamide;

(12) 3-benzyl-N-(4-chlorobenzyl)-9-(tetrahydro-2H-pyran-4-ylmethyl)-7-oxo-2,3-dihydro-7H-[1,3,4]oxadiazino[6,5,4-ij]quinoline-6-carboxamide;

(13) N-(4-chlorobenzyl)-9-(3-hydroxyprop-1-ynyl)-3-methyl-7-oxo-2,3-dihydro-7H-[1,3,4]oxadiazino[6,5,4-ij]quinoline-6-carboxamide;

(14) 3-benzyl-N-(4-chlorobenzyl)-9-(3-hydroxy-1-propynyl)-7-oxo-2,3-dihydro-7H-[1,3,4]thiadiazino[6,5,4-ij]quinoline-6-carboxamide;

(15) 3-benzyl-N-(4-chlorobenzyl)-9-(3-hydroxypropyl)-7-oxo-2,3-dihydro-7H-[1,3,4]thiadiazino[6,5,4-ij]quinoline-6-carboxamide;

(16) 3-benzyl-N-(4-chlorobenzyl)-9-(4-morpholinylmethyl)-7-oxo-2,3-dihydro-7H-[1,3,4]thiadiazino[6,5,4-ij]quinoline-6-carboxamide;

(17) 3-benzyl-N-(4-chlorobenzyl)-9-(tetrahydro-2H-pyran-4-ylmethyl)-7-oxo-2,3-dihydro-7H-[1,3,4]thiadiazino[6,5,4-ij]quinoline-6-carboxamide;

(18) N-(4-chlorobenzyl)-3-methyl-9-(morpholin-4-ylmethyl)-7-oxo-7H-[1,4]thiazino[2,3,4-ij]quinoline-6-carboxamide;

(19) N-(4-chlorobenzyl)-3-methyl-7-oxo-9-(tetrahydro-2H-pyran-4-ylmethyl)-7H-[1,4]thiazino[2,3,4-ij]quinoline-6-carboxamide;

(20) N-(4-chlorobenzyl)-9-(3-hydroxypropyl)-3-methyl-7-oxo-7H-[1,4]thiazino[2,3,4-ij]quinoline-6-carboxamide;

(21) N-(4-chlorobenzyl)-9-(3-hydroxyprop-1-ynyl)-3-methyl-7-oxo-7H-[1,4]thiazino[2,3,4-ij]quinoline-6-carboxamide;

(22) N-(4-chlorobenzyl)-9-(morpholin-4-ylmethyl)-7-oxo-2-pyridin-3-yl-7H-[1,4]thiazino[2,3,4-ij]quinoline-6-carboxamide;

(23) N-(4-chlorobenzyl)-9-(3-hydroxy-1-propynyl)-7-oxo-3-phenyl-1H,7H-[1,3]oxazino[5,4,3-ij]quinoline-6-carboxamide;

(24) N-(4-chlorobenzyl)-9-(3-hydroxypropyl)-7-oxo-3-phenyl-1H,7H-[1,3]oxazino[5,4,3-ij]quinoline-6-carboxamide;

(25) N-(4-chlorobenzyl)-7-oxo-3-phenyl-9-(tetrahydro-2H-pyran-4-ylmethyl)-1H,7H-[1,3]oxazino[5,4,3-ij]quinoline-6-carboxamide;

(26) N-(4-chlorobenzyl)-9-(4-morpholinylmethyl)-7-oxo-3-phenyl-1H,7H-[1,3]oxazino[5,4,3-ij]quinoline-6-carboxamide;

(27) N-(4-chlorobenzyl)-9-(3-hydroxy-1-propynyl)-1-methyl-7-oxo-3-phenyl-2,3-dihydro-1H,7H-pyrido[1,2,3-de]quinoxaline-6-carboxamide;

(28) N-(4-chlorobenzyl)-9-(3-hydroxypropyl)-1-methyl-7-oxo-3-phenyl-2,3-dihydro-1H,7H-pyrido[1,2,3-de]quinoxaline-6-carboxamide;

(29) N-(4-chlorobenzyl)-9-(tetrahydro-2H-pyran-4-ylmethyl)-1-methyl-7-oxo-3-phenyl-2,3-dihydro-1H,7H-pyrido[1,2,3-de]quinoxaline-6-carboxamide;

(30) N-(4-chlorobenzyl)-9-(4-morpholinylmethyl)-1-methyl-7-oxo-3-phenyl-2,3-dihydro-1H,7H-pyrido[1,2,3-de]quinoxaline-6-carboxamide;

(31) N-(4-chlorobenzyl)-9-(3-hydroxy-1-propynyl)-2,3,7-trioxo-2,3-dihydro-1H,7H-pyrido[1,2,3-de]quinoxaline-6-carboxamide;

(32) N-(4-chlorobenzyl)-9-(3-hydroxypropyl)-2,3,7-trioxo-2,3-dihydro-1H,7H-pyrido[1,2,3-de]quinoxaline-6-carboxamide;

(33) N-(4-chlorobenzyl)-9-(4-morpholinylmethyl)-2,3,7-trioxo-2,3-dihydro-1H,7H-pyrido[1,2,3-de]quinoxaline-6-carboxamide;

(34) N-(4-chlorobenzyl)-9-(3-hydroxy-1-propynyl)-2,7-dioxo-2,3-dihydro-1H,7H-pyrido[1,2,3-de]quinoxaline-6-carboxamide;

(35) N-(4-chlorobenzyl)-9-(3-hydroxypropyl)-2,7-dioxo-2,3-dihydro-1H,7H-pyrido[1,2,3-de]quinoxaline-6-carboxamide;

(36) N-(4-chlorobenzyl)-9-(3-hydroxy-1-propynyl)-3,7-dioxo-2,3-dihydro-1H,7H-pyrido[1,2,3-de]quinoxaline-6-carboxamide;

(37) N-(4-chlorobenzyl)-9-(3-hydroxypropyl)-3,7-dioxo-2,3-dihydro-1H,7H-pyrido[1,2,3-de]quinoxaline-6-carboxamide;

(38) N-(4-chlorobenzyl)-9-(4-morpholinylmethyl)-3,7-dioxo-2,3-dihydro-1H,7H-pyrido[1,2,3-de]quinoxaline-6-carboxamide;

(39) N-(4-chlorobenzyl)-9-(4-morpholinylmethyl)-2,7-dioxo-2,3-dihydro-1H,7H-pyrido[1,2,3-de]quinoxaline-6-carboxamide;

(40) N-(4-chlorobenzyl)-9-(3-hydroxy-1-propynyl)-1-methyl-2,7-dioxo-2,3-dihydro-1H,7H-pyrido[1,2,3-de]quinoxaline-6-carboxamide;

(41) 3-benzyl-N-(4-chlorobenzyl)-9-(3-hydroxy-1-propynyl)-1-methyl-7-oxo-2,3-dihydro-1H,7H-[1,2,4]triazino[5,6,1-ij]quinoline-6-carboxamide;

(42) 3-benzyl-N-(4-chlorobenzyl)-9-(3-hydroxypropyl)-1-methyl-7-oxo-2,3-dihydro-1H,7H-[1,2,4]triazino[5,6,1-ij]quinoline-6-carboxamide;

(43) 3-benzyl-N-(4-chlorobenzyl)-9-(4-morpholinylmethyl)-1-methyl-7-oxo-2,3-dihydro-1H,7H-[1,2,4]triazino[5,6,1-ij]quinoline-6-carboxamide;

(44) 3-benzyl-N-(4-chlorobenzyl)-9-(tetrahydro-2H-pyran-4-ylmethyl)-1-methyl-7-oxo-2,3-dihydro-1H,7H-[1,2,4]triazino[5,6,1-ij]quinoline-6-carboxamide;

(45) 1-benzyl-N-(4-chlorobenzyl)-5-(3-hydroxy-1-propynyl)-2,7-dioxo-2,3-dihydro-1H,7H-pyrido[3,2,1-ij]cinnoline-8-carboxamide;

(46) 1-benzyl-N-(4-chlorobenzyl)-5-(3-hydroxypropyl)-2,7-dioxo-2,3-dihydro-1H,7H-pyrido[3,2,1-ij]cinnoline-8-carboxamide;

(47) 1-benzyl-N-(4-chlorobenzyl)-5-(4-morpholinylmethyl)-2,7-dioxo-2,3-dihydro-1H,7H-pyrido[3,2,1-ij]cinnoline-8-carboxamide;

(48) 1-benzyl-N-(4-chlorobenzyl)-5-(tetrahydro-2H-pyran-4-ylmethyl)-2,7-dioxo-2,3-dihydro-1H,7H-pyrido[3,2,1-ij]cinnoline-8-carboxamide;

(49) N-(4-Chlorobenzyl)-5-(3-hydroxyprop-1-ynyl)-1-methyl-7-oxo-2,3-dihydro-1H,7H-pyrido[3,2,1-ij]cinnoline-8-carboxamide;

(50) N-(4-chlorobenzyl)-9-(3-hydroxy-1-propynyl)-7-oxo-3-phenyl-1H,7H-[1,3]thiazino[5,4,3-ij]quinoline-6-carboxamide;

(51) N-(4-chlorobenzyl)-9-(3-hydroxypropyl)-7-oxo-3-phenyl-1H,7H-[1,3]thiazino[5,4,3-ij]quinoline-6-carboxamide;

(52) N-(4-chlorobenzyl)-9-(tetrahydro-2H-pyran-4-ylmethyl)-7-oxo-3-phenyl-1H,7H-[1,3]thiazino[5,4,3-ij]quinoline-6-carboxamide;

(53) N-(4-chlorobenzyl)-9-(4-morpholinylmethyl)-7-oxo-3-phenyl-1H,7H-[1,3]thiazino[5,4,3-ij]quinoline-6-carboxamide;

(54) N-(4-chlorobenzyl)-9-(3-hydroxypropyl)-2-methyl-7-oxo-3-phenyl-2,3-dihydro-1H,7H-pyrido[3,2,1-ij]quinazoline-6-carboxamide;

(55) N-(4-chlorobenzyl)-9-(3-hydroxy-1-propynyl)-2-methyl-7-oxo-3-phenyl-2,3-dihydro-1H,7H-pyrido[3,2,1-ij]quinazoline-6-carboxamide;

(56) N-(4-chlorobenzyl)-9-(4-morpholinylmethyl)-2-methyl-7-oxo-3-phenyl-2,3-dihydro-1H,7H-pyrido[3,2,1-ij]quinazoline-6-carboxamide;

(57) N-(4-chlorobenzyl)-9-(tetrahydro-2H-pyran-4-ylmethyl)-2-methyl-7-oxo-3-phenyl-2,3-dihydro-1H,7H-pyrido[3,2,1-ij]quinazoline-6-carboxamide;

(58) 2-benzyl-N-(4-chlorobenzyl)-9-(4-morpholinylmethyl)-7-oxo-2,3-dihydro-1H,7H-pyrido[3,2,1-ij]quinazoline-6-carboxamide;

(59) 2-benzyl-N-(4-chlorobenzyl)-9-(4-morpholinylmethyl)-3,7-dioxo-2,3-dihydro-1H,7H-pyrido[3,2,1-ij]quinazoline-6-carboxamide;

(60) N-(4-chlorobenzyl)-9-(3-hydroxy-1-propynyl)-2-(4-morpholinyl)-7-oxo-7H-[1,3,4]thiadiazino[6,5,4-ij]quinoline-6-carboxamide;

(61) N-(4-chlorobenzyl)-9-(3-hydroxypropyl)-2-(4-morpholinyl)-7-oxo-7H-[1,3,4]thiadiazino[6,5,4-ij]quinoline-6-carboxamide;

(62) N-(4-chlorobenzyl)-2-(4-morpholinyl)-9-(4-morpholinylmethyl)-7-oxo-7H-[1,3,4]thiadiazino[6,5,4-ij]quinoline-6-carboxamide;

(63) N-(4-chlorobenzyl)-2-(4-morpholinyl)-7-oxo-9-(tetrahydro-2H-pyran-4-ylmethyl)-7H-[1,3,4]thiadiazino[6,5,4-ij]quinoline-6-carboxamide;

(64) N-(4-chlorobenzyl)-9-(3-hydroxypropyl)-7-oxo-2,3-dihydro-1H,7H-pyrazino[3,2,1-ij][1,7]naphthyridine-6-carboxamide;

(65) N-(4-chlorobenzyl)-9-(4-morpholinylmethyl)-7-oxo-2,3-dihydro-1H,7H-pyrazino[3,2,1-ij][1,7]naphthyridine-6-carboxamide;

(66) N-(4-chlorobenzyl)-9-(3-hydroxy-1-propynyl)-7-oxo-2,3-dihydro-1H,7H-pyrazino[3,2,1-ij][1,7]naphthyridine-6-carboxamide;

(67) N-(4-chlorobenzyl)-9-(3-hydroxypropyl)-7-oxo-2,3-dihydro-7H-[1,4]thiazino[2,3,4-ij][1,7]naphthyridine-6-carboxamide;

(68) N-(4-chlorobenzyl)-9-(4-morpholinylmethyl)-7-oxo-2,3-dihydro-7H-[1,4]thiazino[2,3,4-ij][1,7]naphthyridine-6-carboxamide;

(69) N-(4-chlorobenzyl)-9-(3-hydroxy-1-propynyl)-7-oxo-2,3-dihydro-7H-[1,4]thiazino[2,3,4-ij][1,7]naphthyridine-6-carboxamide;

(70) N-(4-chlorobenzyl)-9-(3-hydroxy-1-propynyl)-7-oxo-2,3-dihydro-7H-[1,4]oxazino[2,3,4-ij][1,7]naphthyridine-6-carboxamide;

(71) N-(4-chlorobenzyl)-9-(4-morpholinylmethyl)-7-oxo-2,3-dihydro-7H-[1,4]oxazino[2,3,4-ij][1,7]naphthyridine-6-carboxamide;

(72) N-(4-chlorobenzyl)-9-(3-hydroxypropyl)-7-oxo-2,3-dihydro-7H-[1,4]oxazino[2,3,4-ij][1,7]naphthyridine-6-carboxamide;

(73) N-(4-chlorobenzyl)-9-(3-hydroxypropyl)-3,7-dioxo-3H,7H-pyrido[1,2,3-de]quinoxaline-6-carboxamide;

(74) N-(4-chlorobenzyl)-9-(3-hydroxy-1-propynyl)-3,7-dioxo-3H,7H-pyrido[1,2,3-de]quinoxaline-6-carboxamide;

(75) N-(4-chlorobenzyl)-9-(4-morpholinylmethyl)-3,7-dioxo-3H,7H-pyrido[1,2,3-de]quinoxaline-6-carboxamide;

(76) N-(4-chlorobenzyl)-2-[(4-chlorobenzyl)amino]-9-(4-morpholinylmethyl)-7-oxo-3H,7H-pyrido[1,2,3-de]quinoxaline-6-carboxamide;

(77) 2-(benzylamino)-N-(4-chlorobenzyl)-9-(4-morpholinylmethyl)-7-oxo-3H,7H-pyrido[1,2,3-de]quinoxaline-6-carboxamide;

(78) N-(4-chlorobenzyl)-10-(3-hydroxypropyl)-2,4,8-trioxo-1,2,3,4-tetrahydro-8H-[1,4]diazepino[3,2,1-ij]quinoline-7-carboxamide;

(79) N-(4-chlorobenzyl)-10-(3-hydroxy-1-propynyl)-2,4,8-trioxo-1,2,3,4-tetrahydro-8H-[1,4]diazepino[3,2,1-ij]quinoline-7-carboxamide;

(80) N-(4-chlorobenzyl)-10-(4-morpholinylmethyl)-2,4,8-trioxo-1,2,3,4-tetrahydro-8H-[1,4]diazepino[3,2,1-ij]quinoline-7-carboxamide;

(90) N-(4-chlorobenzyl)-9-(3-hydroxypropyl)-3-methyl-7-oxo-7H-[1,4]oxazino[2,3,4-ij]quinoline-6-carboxamide;

(91) N-(4-chlorobenzyl)-9-(3-hydroxyprop-1-ynyl)-3-methyl-7-oxo-7H-[1,4]oxazino [2,3,4-ij]quinoline-6-carboxamide;

(92) N-(4-chlorobenzyl)-9-(morpholin-4-ylmethyl)-7-oxo-2-pyridin-3-yl-7H-[1,4]oxazino[2,3,4-ij]quinoline-6-carboxamide;

(93) 2-benzyl-N-(4-chlorobenzyl)-10-(4-morpholinylmethyl)-3,8-dioxo-1,2,3,4-tetrahydro-8H-[1,4]diazepino[6,7,1-ij]quinoline-7-carboxamide;

(94) N-(4-chlorobenzyl)-5-(3-hydroxypropyl)-4,7-dioxo-1,2-dihydro-4H,7H-imidazo[1,2,3-ij][1,8]naphthyridine-8-carboxamide;

(95) N-(4-chlorobenzyl)-5-(4-morpholinylmethyl)-4,7-dioxo-1,2-dihydro-4H,7H-imidazo[1,2,3-ij][1,8]naphthyridine-8-carboxamide;

(96) N-(4-chlorobenzyl)-5-(3-hydroxy-1-propynyl)-4,7-dioxo-1,2-dihydro-4H,7H-imidazo[1,2,3-ij][1,8]naphthyridine-8-carboxamide;

(97) N-(4-chlorobenzyl)-6-(4-morpholinylmethyl)-3-oxo-9,10-dihydro-3H,8H-pyrido [3,2,1-ij]quinoline-2-carboxamide;

(98) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide; and pharmaceutically acceptable salts thereof.

A method of preventing or treating atherosclerosis or restenosis in a mammal, wherein the compound administered has the Formula XV and is selected from the group consisting of:

(1) N-(4-chlorobenzyl)-10-(4-morpholinylmethyl)-8-oxo-3,4-dihydro-2H,8H-[1,4]oxazepino [2,3,4-ij]quinoline-7-carboxamide;

(2) N-(4-chlorobenzyl)-8-(4-morpholinylmethyl)-2,6-dioxo-1,2-dihydro-6H-imidazo[4,5,1-ij]quinoline-5-carboxamide;

(3) N-(4-chlorobenzyl)-9-(4-morpholinylmethyl)-2,7-dioxo-2,3-dihydro-1H,7H-pyrido[1,2,3-de]quinoxaline-6-carboxamide;

(4) N-(4-chlorobenzyl)-2-[(4-chlorobenzyl)amino]-9-(4-morpholinylmethyl)-7-oxo-3H,7H-pyrido[1,2,3-de]quinoxaline-6-carboxamide;

(5) N-(4-chlorobenzyl)-2-(hydroxymethyl)-8-(3-hydroxy-1-propynyl)-6-oxo-1,2-dihydro-6H-pyrrolo[3,2,1-ij]quinoline-5-carboxamide;

(6) N-(4-chlorobenzyl)-8-(3-hydroxyprop-1-ynyl)-2,2-dimethyl-6-oxo-1,2-dihydro-6H-pyrrolo[3,2,1-ij]quinoline-5-carboxamide;

(7) N-(4-chlorobenzyl)-6-(morpholin-4-ylmethyl)-3-oxo-9,10-dihydro-3H,8H-pyrido[3,2,1-ij]quinoline-2-carboxamide;

(8) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide;

(9) N-(4-chlorobenzyl)-10-(4-morpholinylmethyl)-2,8-dioxo-1,2,3,4-tetrahydro-8H-[1,4]diazepino[3,2,1-ij]quinoline-7-carboxamide;

(10) 2-(benzylamino)-N-(4-chlorobenzyl)-9-(4-morpholinylmethyl)-7-oxo-3H,7H-pyrido[1,2,3-de]quinoxaline-6-carboxamide;

(11) N-(4-chlorobenzyl)-9-(3-hydroxy-1-propynyl)-1-methyl-2,7-dioxo-2,3-dihydro-1H,7H-pyrido[1,2,3-de]quinoxaline-6-carboxamide;

(12) 2-benzyl-N-(4-chlorobenzyl)-9-(4-morpholinylmethyl)-7-oxo-2,3-dihydro-1H,7H-pyrido[3,2,1-ij]quinazoline-6-carboxamide;

(13) 2-benzyl-N-(4-chlorobenzyl)-9-(4-morpholinylmethyl)-3,7-dioxo-2,3-dihydro-1H,7H-pyrido[3,2,1-ij]quinazoline-6-carboxamide;

(14) 2-benzyl-N-(4-chlorobenzyl)-10-(4-morpholinylmethyl)-3,8-dioxo-1,2,3,4-tetrahydro-8H-[1,4]diazepino[6,7,1-ij]quinoline-7-carboxamide;

(15) N-(4-chlorobenzyl)-9-(3-hydroxyprop-1-ynyl)-3-methyl-7-oxo-2,3-dihydro-7H-[1,3,4]oxadiazino[6,5,4-ij]quinoline-6-carboxamide;

(16) N-(4-Chlorobenzyl)-5-(3-hydroxyprop-1-ynyl)-1-methyl-7-oxo-2,3-dihydro-1H,7H-pyrido[3,2,1-ij]cinnoline-8-carboxamide; and pharmaceutically acceptable salts thereof.

A method of preventing or treating atherosclerosis or restenosis in a mammal, wherein the compound administered has the Formula XV and is selected from the group consisting of:

(1) N-(4-chlorobenzyl)-10-(4-morpholinylmethyl)-8-oxo-3,4-dihydro-2H,8H-[1,4]oxazepino [2,3,4-ij]quinoline-7-carboxamide;

(2) N-(4-chlorobenzyl)-9-(4-morpholinylmethyl)-2,7-dioxo-2,3-dihydro-1H,7H-pyrido[1,2,3-de]quinoxaline-6-carboxamide;

(3) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide;

(4) N-(4-chlorobenzyl)-10-(4-morpholinylmethyl)-2,8-dioxo-1,2,3,4-tetrahydro-8H-[1,4]diazepino[3,2,1-ij]quinoline-7-carboxamide;

(5) 2-(benzylamino)-N-(4-chlorobenzyl)-9-(4-morpholinylmethyl)-7-oxo-3H,7H-pyrido[1,2,3-de]quinoxaline-6-carboxamide;

(6) N-(4-chlorobenzyl)-9-(3-hydroxy-1-propynyl)-1-methyl-2,7-dioxo-2,3-dihydro-1H,7H-pyrido[1,2,3-de]quinoxaline-6-carboxamide; and pharmaceutically acceptable salts thereof.

A method of preventing or treating atherosclerosis or restenosis in a mammal, wherein the compound administered has the Formula XV and is selected from the group consisting of:

(1) N-(4-chlorobenzyl)-9-(4-morpholinylmethyl)-2,7-dioxo-2,3-dihydro-1H,7H-pyrido[1,2,3-de]quinoxaline-6-carboxamide;

(2) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide; and pharmaceutically acceptable salts thereof.

A method for preventing or treating atherosclerosis or restenosis in a mammal, wherein the compound administered has the Formula XV and wherein said mammal is a human.

A method for preventing or treating atherosclerosis or restenosis in a mammal, wherein the compound administered has the Formula XV and wherein said mammal is a livestock or companion animal.

A method for preventing or treating atherosclerosis or restenosis in a mammal, wherein the compound administered has the Formula XV and wherein the amount administered is from about 0.1 to about 300 mg/kg of body weight.

A method for preventing or treating atherosclerosis or restenosis in a mammal, wherein the compound administered has the Formula XV and wherein the amount administered is from about 1.0 to about 30 mg/kg of body weight.

A method for preventing or treating atherosclerosis or restenosis in a mammal, wherein the compound administered has the Formula XV and wherein the compound is administered parenterally, topically, intravaginally, orally, or rectally.

In the definitions of the substitutents of Formulae X, XII, XIII, XIV and XV, it is to be understood that the prefixed numbered R groups correspond to numbered R groups of the W Formulae.

Dosages and Pharmaceutical Compositions

By the term “effective amount” of a compound as provided herein is meant a nontoxic but sufficient amount of one or more anti-atherosclerosis or anti-restenosis agents to provide the desired effect. The desired effect may be to prevent, give relief from, or ameliorate atherosclerosis or restenosis.

As pointed out below, the exact amount of the anti-atherosclerosis or anti-restenosis agent required to treat atherosclerosis or restenosis will vary from subject to subject, depending on the species, age, and general condition of the subject, the severity of the disease that is being treated, the particular compound(s) used, the mode of administration, such as the route and frequency of administration, and the particular compound(s) employed, and the like. Thus, it is not possible to specify an exact “effective amount.” However, an appropriate effective amount may be determined by one of ordinary skill in the art using only routine experimentation.

Pharmaceutical compositions including one or more anti-atherosclerosis or anti-restenosis agents can be administered orally or parenterally at dose levels, calculated as the free base, of each of the anti-atherosclerosis or anti-restenosis agent at 0.1 to 300 mg/kg, preferably 1.0 to 30 mg/kg of mammal body weight, and can be used in a human in a unit dosage form, administered one to four times daily in the amount of 1 to 1000 mg per unit dose. The desired dosage may conveniently be presented in a single dose or as divided into multiple doses administered at appropriate intervals, for example, as two, three, four or more sub-doses per day. The sub-dose itself may be further divided, e.g., into a number of discrete loosely spaced administrations.

Initial treatment of a patient suffering from atherosclerosis or restenosis can begin with a dosage regimen as indicated above. Treatment is generally continued as necessary over a period of several weeks to several months or years until the condition or disorder has been controlled or eliminated. Patients undergoing treatment with a composition of the invention can be routinely monitored by any of the methods well known in the art to determine the effectiveness of therapy. Continuous analysis of data from such monitoring permits modification of the treatment regimen during therapy so that optimally effective amounts of drug are administered at any point in time, and so that the duration of treatment can be determined. In this way, the treatment regimen and dosing schedule can be rationally modified over the course of therapy so that the lowest amount of the compounds of this invention exhibiting satisfactory effectiveness is administered, and so that administration is continued only for so long as is necessary to successfully treat the condition or disorder.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

In a combination therapy, the anti-atherosclerosis or anti-restenosis agent compound(s) and other inhibitor compound(s) can be administered simultaneously or at separate intervals. When administered simultaneously the anti-atherosclerosis or anti-restenosis agent compound(s) and the other inhibitor compound(s) can be incorporated into a single pharmaceutical composition or into separate compositions, e.g., anti-atherosclerosis or anti-restenosis agent compound(s) in one composition and the other inhibitor compound(s) in another composition. For instance the combination therapy, the anti-atherosclerosis or anti-restenosis agent compound(s) may be administered concurrently or concomitantly with the other inhibitor compound(s). The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

When separately administered, therapeutically effective amounts of anti-atherosclerosis or anti-restenosis agent compound(s) and the other inhibitor compound(s) are administered on a different schedule. One may be administered before the other as long as the time between the two administrations falls within a therapeutically effective interval. A therapeutically effective interval is a period of time beginning when one of either (a) the anti-atherosclerosis or anti-restenosis agent compound(s), or (b) the other inhibitor compound(s) is administered to a mammal and ending at the limit of the beneficial effect in the treatment of atherosclerosis or restenosis of the combination of (a) and (b). The methods of administration of the anti-atherosclerosis or anti-restenosis agent compounds and the other inhibitor compound(s) may vary. Thus, one agent may be administered orally, while the other is administered by injection.

A specific active agent may have more than one recommended dosage range, particularly for different routes of administration. Generally, an effective amount of dosage of anti-atherosclerosis or anti-restenosis agent compound(s), either administered individually or in combination with other inhibitor compound(s), will be in the range of about 0.1 to about 300 mg/kg of body weight/day, preferably from 1 to 30 mg/kg of body weight. It is to be understood that the dosages of active component(s) may vary depending upon the requirements of each subject being treated and the severity of the atherosclerosis or restenosis.

In addition to the anti-atherosclerosis or anti-restenosis agents, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Generally, the concentration of each of the anti-atherosclerosis or anti-restenosis agents in a liquid composition, such as a lotion, will be from about 0.1 wt. % to about 20 wt. %, preferably from about 0.5 wt. % to about 10 wt. %. The solution may contain other ingredients, such as emulsifiers, antioxidants or buffers. The concentration in a semi-solid or solid composition, such as a gel or a powder will be about 0.1 wt. % to about 5 wt. %, preferably about 0.5 wt. % to about 2.5 wt. %. When the topically deliverable, pharmaceutical composition of the present invention is utilized to effect targeted treatment of a specific internal site, each of the anti-atherosclerosis or anti-restenosis agent is preferably contained in the composition in an amount of from 0.05-10 wt. %, more preferably 0.5-5 wt. %.

Routes of Administration

In therapeutic use for treating or preventing atherosclerosis or restenosis in a mammal (i.e. human and animals) the pharmaceutical composition including the anti-atherosclerosis or anti-restenosis agent(s) can be administered orally, parenterally, topically, rectally, or intranasally.

Parenteral administrations include injections to generate a systemic effect or injections directly to the afflicted area. Examples of parenteral administrations are subcutaneous, intravenous, intramuscular, intradermal, intrathecal, intraocular, intravetricular, and general infusion techniques.

Topical administrations include the treatment of infectious areas or organs readily accessible by local application, such as, for example, eyes, ears including external and middle ear infections, vaginal, open and sutured or closed wounds and skin. It also includes transdermal delivery to generate a systemic effect.

The rectal administration includes the form of suppositories.

The intranasally administration includes nasal aerosol or inhalation applications.

Pharmaceutical compositions including the anti-atherosclerosis or anti-restenosis agent(s) may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the anti-atherosclerosis or anti-restenosis agent(s) can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identificatin or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of pharmaceutical compositions with the anti-atherosclerosis or anti-restenosis agent(s) dissolved in water and water-propylene glycol and water-polyethylene glycol systems, optionally containing suitable conventional coloring agents, flavoring agents, stabilizers and thickening agents.

The anti-atherosclerosis or anti-restenosis agent(s) may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the anti-atherosclerosis or anti-restenosis agent(s) may be formulated in aqueous solution, preferably in physiologically compatible buffers or physiological saline buffer. Suitable buffering agents include tri-sodium orthophosphate, sodium bicarbonate, sodium citrate, N-methyl-glucamine, L(+)-lysine and L(+)-arginine.

The compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the anti-atherosclerosis or anti-restenosis agent(s). Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the anti-atherosclerosis or anti-restenosis agent(s) may be in a powder form for constitution with a suitable vehicle, e.g., sterile, pyrogen-free water, before use.

For suppository administration, the pharmaceutical compositions may also be formulated by mixing the anti-atherosclerosis or anti-restenosis agent(s) with a suitable non-irritating excipient which is solid at room temperature but liquid at rectal temperature and therefore will melt in the rectum to release the drug. Such materials include cocoa butter, beeswax and other glycerides.

For administration by inhalation, the anti-atherosclerosis or anti-restenosis agent(s) can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a powder base such as lactose or starch.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative, such as benzylalkonium chloride. Alternatively, for ophthalmic uses, the pharmaceutical compositions may be formulated in an ointment, such as petrolatum.

In addition to the formulations described previously, the anti-atherosclerosis or anti-restenosis agent(s) may also be formulated as depot preparations. Such long acting formulations may be in the form of implants. The anti-atherosclerosis or anti-restenosis agent(s) may be formulated for this route of administration with suitable polymers, hydrophobic materials, or as a sparing soluble derivative such as, without limitation, a sparingly soluble salt.

Additionally, the anti-atherosclerosis or anti-restenosis agent(s) may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

In certain embodiments, the anti-atherosclerosis or anti-restenosis agent(s) are applied topically. For topical applications, the pharmaceutical composition may be formulated in a suitable ointment containing the anti-atherosclerosis or anti-restenosis agent(s) suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

Several different animal models are available to evaluate reduction of atherosclerosis or restinosis by antiviral drug treatment. In these models histological changes in the atherosclerotic lesions of aortic arteries are measured in animals infected with a herpesvirus and treated or untreated with an antiviral drug capable of inhibiting replication of the herpesvirus. The models include murine CMV infection of apoE deficient mice and rat CMV infection of rats. These models would mimic the effects of human CMV infection. MHV-68 is a murine gammaherpesvirus related to EBV. Antiviral treatment has been shown to reduce atherosclerosis caused by HMV-68 infection in apoE deficient mice. Drugs containing compounds of Formula X and XII-XV inhibit replication of these animal viruses so the models could be used to show an effect of drugs containing compounds of Formula I and II on development of atherosclerosis. Lemstrom, et al, “Cytomegalovirus Infection-Enhanced Allograft Atherosclerosis is prevented by DHPG Prophylaxis in the Rat”, Circulation Vol. 90, No. 4, October 1994, pp 1969-1978; Burnell et al, “Atherosclerosis in a poE Knockout Mice Infected with Multiple Pathogens”. Both of these references are herein incorporated by reference.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited by the claims which follow.

All published documents are incorporated by reference herein.

In claims to methods utilizing compounds of Formula XII, XIII, XIV and XV, it is to be understood that the prefixed numbered R groups correspond to numbered R Groups of the W Formulae.

What is claimed is:

1. A method of preventing or treating atherosclerosis or restenosis in a mammal, comprising administering to said mammal an effective amount of a compound selected from the group consisting of structures of Formula X, Formula XII, Formula XIII, Formula XIV and Formula XV,
wherein Formula X is 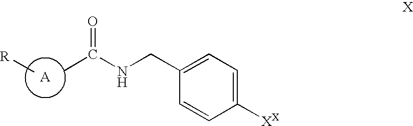
and pharmaceutically acceptable salts thereof; and
wherein ring A is a saturated or unsaturated fused double or triple heterocyclic ring having 1, 2, 3 or 4 heteroatoms selected from group consisting of oxygen, sulfur, or nitrogen; and
wherein R and Xx are the appropriated substitutents, respectively;
wherein Formula XII is 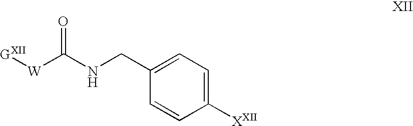
wherein,
X^{XII} is Cl, Br, F, CN or NO₂;
G^{XII} is
a) C₁₋₇alkyl which partially unsaturated and is substituted by hydroxy, or
b) C₁₋₄alkyl substituted by NR^{XII-1}R^{XII-2} or 4-tetrahydropyran;
R^{XII-1} is C₂₋₇alkyl substituted by hydroxy, C₁₋₄alkoxy, aryl^{XII}, or heteroaryl;
R^{XII-2} is hydrogen or C₁₋₇alkyl; or
R^{XII-1} and R^{XII-2} together with the nitrogen to which they are attached form morpholine which may be optionally substituted by ^{XII-}aryl or C₁₋₇alkyl;
W^{XII} is a heterocycle of formula W1^{XII}, W3^{XII}, or W4^{XII}; 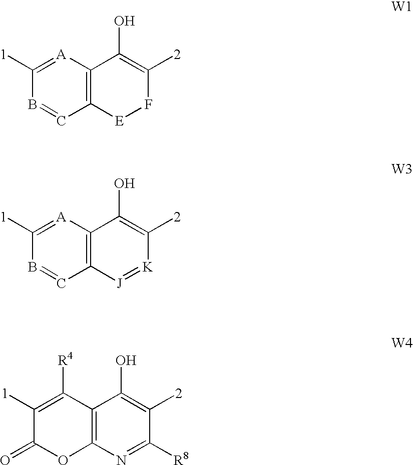
[It is intended that the R⁴ and R⁸ substituent and substituents A-F, J and K on the W1^{XII}, W3^{XII}, and W4^{XII} Formulae above, as well as the W numbers under the formulas, be succeeded by the Roman Numeral XII.]
A^{XII} is CR^{XII-4} or nitrogen;
B^{XII} is CR^{XII-5} or nitrogen;
C^{XII} is C^{RXII-6} or nitrogen;
E^{XII} and F^{XII} are such that
a) one is oxygen and the other is C(═O); or
b) E^{XII} is C(═O) and F^{XII} is NR^{XII-7};
J^{XII} and K^{XII} are such that
a) J^{XII} is nitrogen and K^{XII} is CR^{XII-8}; or
b) J^{XII} is CR^{XII-6} and K^{XII} is nitrogen;
with the provisos that when W^{XII} is of formula W3^{XII} and
J^{XII} is nitrogen, then at least one of A^{XII} and B^{XII} is nitrogen;
R^{XII-4} is H, halogen, or C₁₋₄alkyl optionally substituted by one to three halogens;
R^{XII-5} is
a) H,
b) halo,
c) OR^{XII-12},
d) SR^{XII-12},
e) C₁₋₇alkyl which may be partially unsaturated and optionally substituted by one or more substituents selected from OR^{XII-12}, SR^{XII-12}, NR^{XII-10}R^{XII-11}, or halo,
f) C₃₋₈cycloalkyl which may be partially unsaturated and is optionally substituted by one or more substituents selected from halogen, OR^{XII-12}, SR^{XII-12}, or NR^{XII-10}R^{XII-11},
g) (C═O)R^{XII-9},
h) S(O)ₘ^{XII}R^{XII-9},
i) (C═O)OR^{XII-2},
j) NHSO₂R^{XII-9},
k) nitro, or
l) cyano;
R^{XII-6} is
a) H,
b) halo,
c) aryl^{XII},
d) het^{XII},
e) OR^{XII-12},
f) SR^{XII-2},
g) C₁₋₇alkyl which may be partially unsaturated and optionally substituted by one or more substituents selected from OR^{XII-12}, SR^{XII-12}, NR^{XII-10}R^{XII-11}, aryl^{XII}, halo, C₃₋₈cycloalkyl optionally substituted by OR¹², or het^{XII}, attached through a carbon atom,
h) NR^{XII-10}R^{XII-11},
i) C₃₋₈cycloalkyl which may be partially unsaturated and is optionally substituted by one or more substituents selected from halogen, OR^{XII-12}, SR^{XII-12}, or NR^{XII-10}R^{XII-11},
j) (C═O)R^{XII-9},
k) S(O)ₘ^{XII}R^{XII-9},
l) (C═O)OR^{XII-2},
m) NHSO₂R^{XII-9},
n) nitro, or
o) cyano;
R^{XII-7} is
a) H,
b) C₁₋₇alkyl which may be partially unsaturated and optionally substituted by one or more substituents selected from OR^{XII-12}, SR^{XII-12}, NR^{XII-10}R^{XII-11}, or halo,
c) C₃₋₈cycloalkyl which may be partially unsaturated and is optionally substituted by one or more substituents selected from halogen, XII-12 XII-12 or 10 XI1 OR^{XII-12}, SR^{XII-12}, or NR^{XII-10}R^{XII-11},
d) aryl^{XII}, or
e) het^{XII};
R^{XII-8} is
a) H,
b) C₁₋₇alkyl which may be partially unsaturated and optionally substituted by one or more substituents selected from OR^{XII-12}, SR^{XII-12}, NR^{XII-10}R^{XII-11}, or halo,
c) OR^{XII-12}, or
d) SR^{XII-12};
R^{XII-9} is
a) C₁₋₇alkyl,
b) NR^{XII-10}R^{XII-11},
c) aryl^{XII}, or
d) het^{XII}, wherein said het^{XII} is bound through a carbon atom;
R^{XII-10} and R^{XII-11} are independently
a) H,
b) aryl^{XII},
c) C₁₋₇alkyl which may be partially unsaturated and is optionally substituted by one or more substituents selected from CONR²R², CO₂R², het^{XII}, aryl^{XII}, cyano, or halo,
d) C₂₋₇alkyl which may be partially unsaturated and is substituted by one or more substituents selected from NR^{XII-2}R^{XII-2}, OR^{XII-2}, or SR^{XII-2},
e) C₃₋₈cycloalkyl which may be partially unsaturated and is optionally substituted by one or more substituents selected from halogen, OR^{XII-2}, SR^{XII-2}, or NR^{XII-2}R^{XII-2}, or
f) R^{XII-10} and R^{XII-11} together with the nitrogen to which they are attached form a het^{XII};
R^{XII-12} is
a) H,
b) aryl^{XII},
c) het^{XII},
d) C₁₋₇alkyl optionally substituted by aryl^{XII}, het^{XII}, or halogen,
e) C₂₋₇alkyl substituted by OR^{XII-2}, SR^{XII-2}, or NR^{XII-2}R^{XII-2}, or
f) C₃₋₈cycloalkyl which may be partially unsaturated and is optionally substituted by one or more substituents selected from halogen, OR^{XII-2}, SR^{XII-2}, or NR^{XII-2}R^{XII-2};
each m^{XII} is independently 1 or 2;
aryl^{XII} is a phenyl radical or an ortho-fused bicyclic carbocyclic radical wherein at least one ring is aromatic, and aryl^{XII} maybe optionally substituted with one or more substituents selected from halo, OH, cyano, NR^{XII-2}R^{XII-2}, CO₂R^{XII-2}, CF₃, C₁₋₆alkoxy, and C₁₋₆ alkyl which maybe further substituted by one to three SR^{XII-2}, NR^{XII-2}R^{XII-2}, OR^{XII-2}, or CO₂R^{XII-2} groups;
het^{XII} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XII} may be optionally substituted with one or more substituents selected from halo, OH, cyano, phenyl, CO₂R^{XII-2}, CF₃, C₁₋₆alkoxy, oxo, oxime, and C₁₋₆ alkyl which may be further substituted by one to three SR^{XII-2}, NR^{XII-2}R^{XII-2}, OR^{XII-2}, or CO₂R^{XII-2} groups;
halo or halogen is F, Cl, Br, I;
1 represents the point of attachment between W^{XII} and G^{XII};
2 represents the point of attachment between W^{XII} and the carbonyl group of Formula (I);
and a pharmaceutically acceptable salt thereof;
wherein Formula XIII is 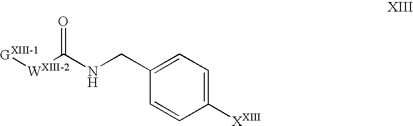
wherein,
X^{XIII} is Cl, Br, F, CN or NO₂;
G^{XIII} is
a) C₃₋₇alkyl which is partially unsaturated and is substituted by hydroxy, or
b) C₁₋₇alkyl substituted by NR^{XIII-1}R^{XIII-2} or 4-tetrahydropyran;
R^{XIII-1} is C₂₋₇alkyl substituted by hydroxy, C₁₋₄alkoxy, aryl^{XII}, or heteroaryl^{XII};
R^{XIII-2} is hydrogen or C₁₋₇alkyl; or
R^{XIII-1} and R^{XIII-2} together with the nitrogen to which they are attached form morpholine which may be optionally substituted by aryl^{XII} or C₁₋₇alkyl;
W^{XIII} is 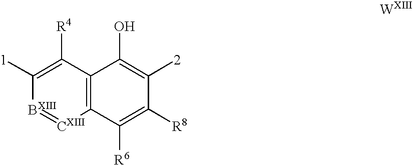
B^{XIII} is CR^{XIII-5}, or nitrogen;
C^{XIII} is CR^{XIII-6} or nitrogen;
with the provisos that B^{XIII} and C^{XIII} cannot be both nitrogen;
R^{XIII-4} is H, halogen, or C₁₋₄alkyl optionally substituted by one to three halogens;
R^{XIII-5} is
a) H,
b) halo,
c) OR^{XIII-12},
d) SR^{XIII-12},
e) C₁₋₇alkyl which may be partially unsaturated and optionally substituted by one or more substituents selected from OR^{XIII-12}, SR^{XIII-12}, NR^{XIII-10}R^{XIII-11}, or halo,
f) C₃₋₈cycloalkyl which may be partially unsaturated and is optionally substituted by one or more substituents selected from halogen, OR^{XIII-12}, SR^{XIII-12}, or NR^{XIII-10}R^{XIII-11},
g) (C═O)R^{XIII-9},
h) S(O)ₘ^{XIII}R^{XIII-9},
i) (C═O)OR^{XIII-2},
j) NHSO₂R^{XIII-9},
k) nitro, or
l) cyano;
R^{XIII-6} is
a) H,
b) halo,
c) aryl^{XIII},
d) het^{XIII}, or
e) R^{XIII-7};
R^{XIII-7} is
a) OR^{XIII-12},
b) SR^{XIII-12},
c) C₁₋₇alkyl which may be partially unsaturated and optionally substituted by one or more substituents selected from OR^{XIII-12}, SR^{XIII-12}, NR^{XIII-10}R^{XIII-11}, aryl^{XIII}, halo, C₃₋₈cycloalkyl optionally substituted by OR^{XIII-12}, or het^{XIII} attached through a carbon atom,
d) NR^{XIII-10}R^{XIII-11},
e) C₃₋₈cycloalkyl which may be partially unsaturated and is optionally substituted by one or more substituents selected from halogen, OR^{XIII-12}, SR^{XIII-12}, or NR^{XIII-10}R^{XIII-11},
f) (C═O)R^{XIII-9},
g) S(O)ₘ^{XIII}R^{XIII-9},
h) (C═O)OR^{XIII-2},
i) NHSO₂R^{XIII-9},
j) nitro, or
k) cyano;
R^{XIII-8} is
a) H,
b) C₁₋₇alkyl which may be partially unsaturated and optionally substituted by one or more substituents selected from OR^{XIII-12}, SR^{XIII-12}, NR^{XIII-10}R^{XIII-11}, or halo,
c) OR^{XIII-12}, or
d) SR^{XIII-12},
R^{XII-9} is
a) C₁₋₇alkyl,
b) NR^{XIII-10}R^{XIII-11},
c) aryl^{XIII}, or
d) het^{XIII}, wherein said het^{XIII} is bound through a carbon atom;
R^{XIII-10} and R^{XIII-11} are independently
a) H,
b) aryl^{XIII},
c) C₁₋₇alkyl which may be partially unsaturated and is optionally substituted by one or more substituents selected from CONR^{XIII-2}R^{XIII-2}, CO₂R^{XIII-2}, hetX^{XIII}, aryl^{XIII}, cyano, or halo,
d) C₂₋₇alkyl which may be partially unsaturated and is substituted by one or more substituents selected from NR^{XIII-2}R², OR^{XIII-2}, or SR^{XIII-2},
e) C₃₋₈cycloalkyl which may be partially unsaturated and is optionally substituted by one or more substituents selected from halogen, OR^{XIII-2}, SR^{XIII-2}, or NR^{XIII-2}R^{XIII-2}, or
f) R^{XIII-10} and R^{XIII-11} together with the nitrogen to which they are attached form a het^{XIII};
R^{XIII-12} is
a) H,
b) aryl^{XIII},
c) het^{XIII},
d) C₁₋₇alkyl optionally substituted by aryl^{XIII}, het^{XIII}, or halogen,
e) C₂₋₇alkyl substituted by OR^{XIII-2}, SR^{XIII-2}, or NR^{XIII-2}R^{XIII-2}, or
f) C₃₋₈cycloalkyl which may be partially unsaturated and is optionally substituted by one or more substituents selected from halogen, OR^{XIII-2}, SR^{XIII-2}, or NR^{XIII-2}R^{XII-2};
each m^{XIII} is independently 1 or 2;
aryl^{XIII} is a phenyl radical or an ortho-fused bicyclic carbocyclic radical wherein at least one ring is aromatic, and aryl^{XIII} maybe optionally substituted with one or more substituents selected from halo, OH, cyano, NR^{XIII-2}R^{XIII-2}, CO₂R^{XIII-2}, CF₃, C₁₋₆alkoxy, and C₁₋₆ alkyl which maybe further substituted by one to three SR^{XIII-2}, NR^{XIII-2}R^{XIII-2}, OR^{XIII-2}, or CO₂R^{XIII-2} groups;
het^{XIII} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and ^{XIII-}het may be optionally substituted with one or more substituents selected from halo, OH, cyano, phenyl, CO₂R^{XIII-2}, CF₃, C₁₋₆alkoxy, oxo, oxime, and C₁₋₆ alkyl which may be further substituted by one to three SR^{XIII-2}, NR^{XIII-2}R^{XIII-2}, OR^{XIII-2}, or CO₂R^{XIII-2} groups;
halo or halogen is F, Cl, Br, I;
1 represents the point of attachment between W^{XIII} and G^{XIII};
2 represents the point of attachment between W and the carbonyl group of Formula (I);
and a pharmaceutically acceptable salt thereof;
wherein Formula XIV is 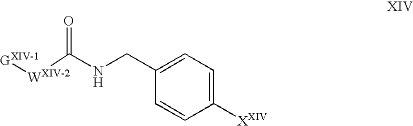
wherein,
X^{XIV} is Cl, Br, F, CN, or NO₂;
G^{XIV} is
a) C₁₋₄alkyl which is fully saturated or partially unsaturated and is substituted by hydroxy, or
b) C₁₋₄alkyl substituted by NR^{XIV-1}R^{XIV-2} or 4-tetrahydropyran;
R^{XIV-1} is C₂₋₇alkyl substituted by hydroxy, C₁₋₄alkoxy, heteroaryl, or aryl^{XIV};
R^{XIV-2} is hydrogen or C₁₋₇alkyl; or
R^{XIV-1} and R^{XIV-2} together with the nitrogen to which they are attached form morpholine which may be optionally substituted by aryl^{XIV} or C₁₋₇alkyl; or pyrrolidine substituted by hydroxy;
W^{XIV} is a heterocycle of formula W1^{XIV}, W2^{XIV}, W3^{XIV}, W4^{XIV}, W5^{XIV}, W6^{XIV}, W7^{XIV}, W8^{XIV}, W9^{XIV}, W10^{XIV}, W11^{XIV}, W12^{XIV}, W13^{XIV}, W14^{XIV}, W15^{XIV}, W16^{XIV}, W17^{XIV}, W18^{XIV}, W19^{XIV}, W20^{XIV}, W21^{XIV} or W22^{XIV} 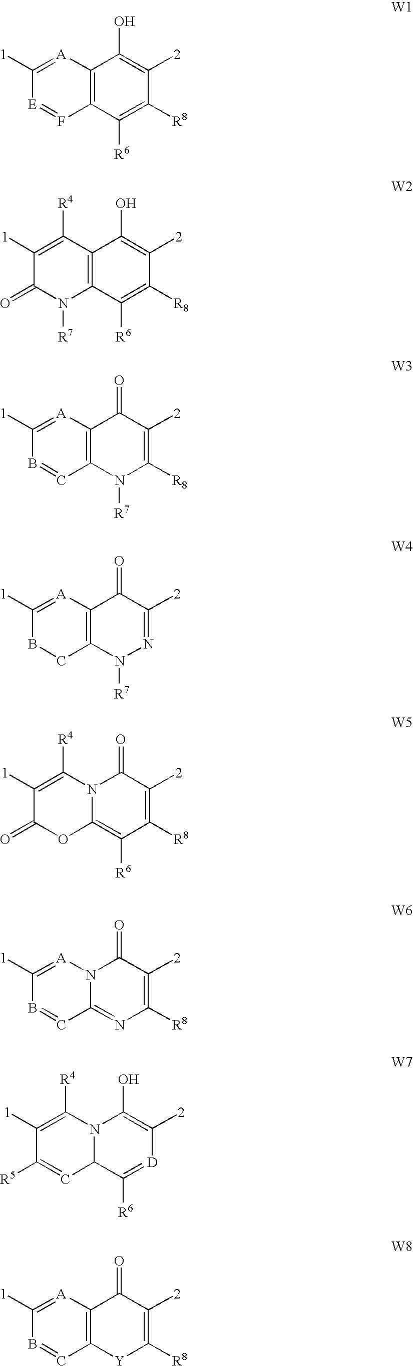 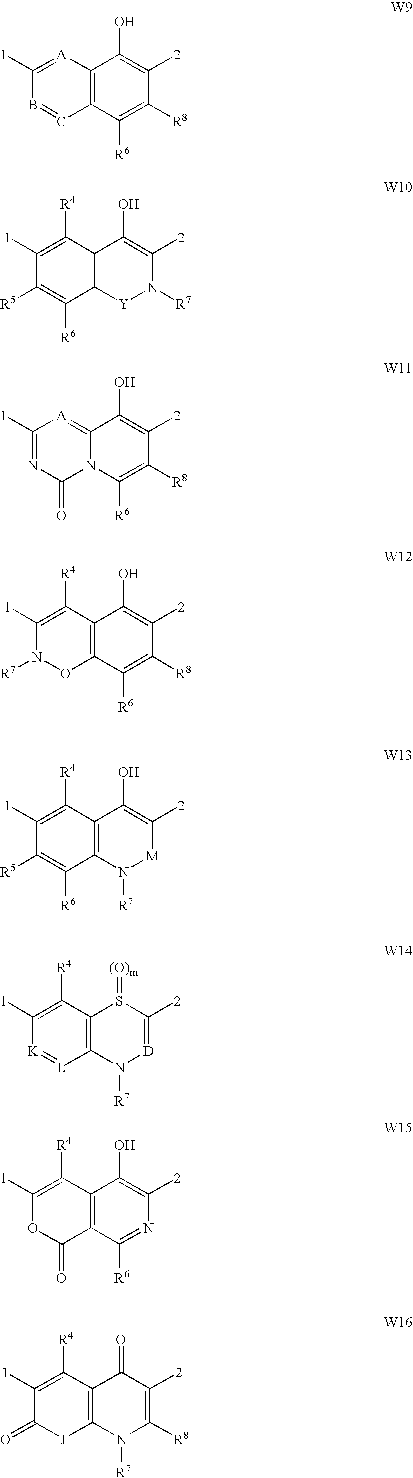 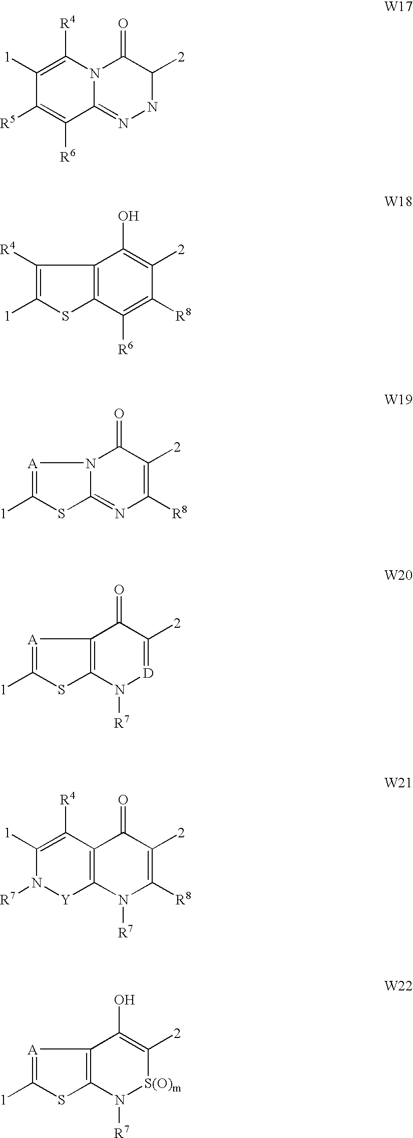
[It is intended that the R substituents and substituents A-F, J, K and Y on the W Formulae above, as well as the W number under the formulas, be succeeded by the Roman Numeral XIV.]
A^{XIV} is CRXIV⁴ or nitrogen;
B^{XIV} is CRXIV-5 or nitrogen;
C^{XIV} is CRXIV-6 or nitrogen;
D^{XIV} is CRXIV-8 or nitrogen;
E^{XIV} and F are such that one is oxygen and the other is C(═O);
J^{XIV} is NR^{XIV-7} or oxygen;
K^{XIV} and L are defined such that
a) K^{XIV} is CR^{XIV-5} and L^{XIV} is CR^{XIV-6}, or
b) K^{XIV} is absent and L^{XIV} is sulfur;
M^{XIV} is oxygen, sulfur, or S(O)ₘ;
Y^{XIV} is oxygen or sulfur;
R^{XIV-4} is H, halogen, or C₁₋₄alkyl optionally substituted by one to three halogens;
R^{XIV-5} is
a) H,
b) halo,
c) OR^{XIV-12},
d) SR^{XIV-12},
e) C₁₋₇alkyl which may be partially unsaturated and optionally substituted by one or more substituents selected from OR^{XIV-12}, SR^{XIV-12}, NR^{XIV-10}R^{XIV-11}, or halo,
f) C₃₋₈cycloalkyl which may be partially unsaturated and is optionally substituted by one or more substituents selected from halogen, OR^{XIV-12}, SR^{XIV-12}, or NR^{XIV-10}R^{XIV-11},
g) (C═O)R^{XIV-9},
h) S(O)ₘ^{XIV}R^{XIV-9},
i) (C═O)OR^{XIV-2},
j) NHSO₂R^{XIV-9},
k) nitro, or
l) cyano;
R^{XIV-6} is
a) H,
b) halo,
c) aryl^{XIV},
d) het^{XIV},
e) OR^{XIV-12},
f) SR^{XIV-12},
g) C₁₋₇alkyl which may be partially unsaturated and optionally substituted by one or more substituents selected from OR^{XIV-12}, SR^{XIV-12}, NR^{XIV-10}R^{XIV-11}, ^{XIV-}aryl, halo, C₃₋₈cycloalkyl optionally substituted by OR^{XIV-12}, or ^{XIV-}het attached through a carbon atom,
h) NR^{XIV-10}R^{XIV-11},
i) C₃₋₈cycloalkyl which may be partially unsaturated and is optionally substituted by one or more substituents selected from halogen, OR^{XIV-}12, SR^{XIV-}12, or NR^{XIV-10}R^{XIV-11},
j) (C═O)R^{XIV-9},
k) S(O)ₘ^{XIV}R^{XIV-9},
l) (C═O)OR^{XIV-2},
m) NHSO₂R^{XIV-9},
n) nitro, or
o) cyano;
R^{XIV-7} is
a) H,
b) C₁₋₇alkyl which may be partially unsaturated and optionally substituted by one or more substituents selected from OR^{XIV-12}, SR^{XIV-12}, NR^{XIV-10}R^{XIV-11}, or halo,
c) C₃₋₈cycloalkyl which may be partially unsaturated and is optionally substituted by one or more substituents selected from halogen, OR^{XIV-12}, SR^{XIV-12}, or NR^{XIV-10}R^{XIV-11},
d) aryl^{XIV}, or
e) het^{XIV};
R^{XIV-8} is
a) H,
b) C₁₋₇alkyl which may be partially unsaturated and optionally substituted by one or more substituents selected from OR^{XIV-12}, SR^{XIV-12}, NR^{XIV-10}R^{XIV-11}, or halo,
c) OR^{XIV-12}, or
d) SR^{XIV-12};
R^{XIV-9} is
a) C₁₋₇alkyl,
b) NR^{XIV-10}R^{XIV-11},
c) aryl^{XIV}, or
d) het^{XIV} wherein said ^{XIV-}het is bound through a carbon atom;
R^{XIV-10} and R^{XIV-11} are independently
a) H,
b) aryl^{XIV},
c) C₁₋₇alkyl which may be partially unsaturated and is optionally substituted by one or more substituents selected from CONR^{XIV-2}R^{XIV-2}, CO₂R^{XIV-2}, het^{XIV}, aryl^{XIV}, cyano, or halo,
d) C₂₋₇alkyl which may be partially unsaturated and is substituted by one or more substituents selected from NR^{XIV-2}R^{XIV-2}, OR^{XIV-2}, or SR^{XIV-2},
e) C₃₋₈cycloalkyl which may be partially unsaturated and is optionally substituted by one or more substituents selected from halogen, OR^{XIV-2}, SR^{XIV-2}, or NR^{XIV-2}R^{XIV-2}, or
f) R^{XIV-10} and R^{XIV-11} together with the nitrogen to which they are attached form a het^{XIV};
R^{XIV-12} is
a) H,
b) aryl^{XIV},
c) het^{XIV}
d) C₁₋₇alkyl optionally substituted by aryl^{XIV}, or halogen,
e) C₂₋₇alkyl substituted by OR^{XIv-2}, SR^{XIV-2}, or NR^{XIV-2}R^{XIV-2}, or
f) C₃₋₈cycloalkyl which may be partially unsaturated and is optionally substituted by one or more substituents selected from halogen, OR^{XIV-2}, SR^{XIV-2}, or NR^{XIV-2}R^{XIV-2};
each m^{XIV} is independently 1 or 2;
aryl^{XIV} is a phenyl radical or an ortho-fused bicyclic carbocyclic radical wherein at least one ring is aromatic, and aryl^{XIV} may be optionally substituted with one or more substituents selected from halo, OH, cyano, NR^{XIV-2}R^{XIV-2}, CO₂R^{XIV-2}, CF₃, C₁₋₆alkoxy, and C₁₋₆ alkyl which maybe further substituted by one to three SR², NR^{XIV-2}R^{XIV-2}, OR^{XIV-2}, or CO₂R^{XIV-2} groups;
het^{XIV} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XIV} may be optionally substituted with one or more substituents selected from halo, OH, cyano, phenyl, CO₂R^{XIV-2}, CF₃, C₁₋₆alkoxy, oxo, oxime, and C₁₋₆ alkyl which may be further substituted by one to three SR^{XIV-2}, NR^{XIV-2}R^{XIV-2}, OR^{XIV-2}, or CO₂R^{XIV-2} groups;
halo or halogen is F, Cl, Br, I;
1 represents the point of attachment between W and G;
2 represents the point of attachment between W and the carbonyl group of Formula (I);
and a pharmaceutically acceptable salt thereof;
wherein Formula XV is 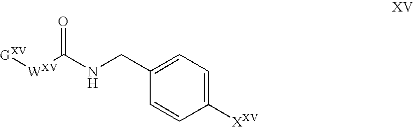
or a pharmaceutically acceptable salt thereof,
wherein,
X^{XV} is Cl, Br, F, CN or NO₂;
G^{XV} is
a) C₁₋₄alkyl which is fully saturated or partially unsaturated and is substituted by hydroxy, or
b) C₁₋₄alkyl substituted by NR^{XV-1}R^{XV-2} or 4-tetrahydropyran;
R^{XV-1} is C₂₋₇alkyl substituted by hydroxy, C₁₋₄alkoxy, or aryl;
R^{XV-2} is hydrogen or C₁₋₇alkyl; or
R^{XV-1} and R^{XV-2} together with the nitrogen to which they are attached form
a) a morpholine which may be optionally substituted by aryl or C₁₋₇alkyl; or
b) a pyrrolidine ring substituted by hydroxy;
W^{XV} is a heterocycle of formula W1^{XV}, W2^{XV}, W3^{XV}, W4^{XV}, W5^{XV}, W6^{XV}, W7^{XV} or W8^{XV} 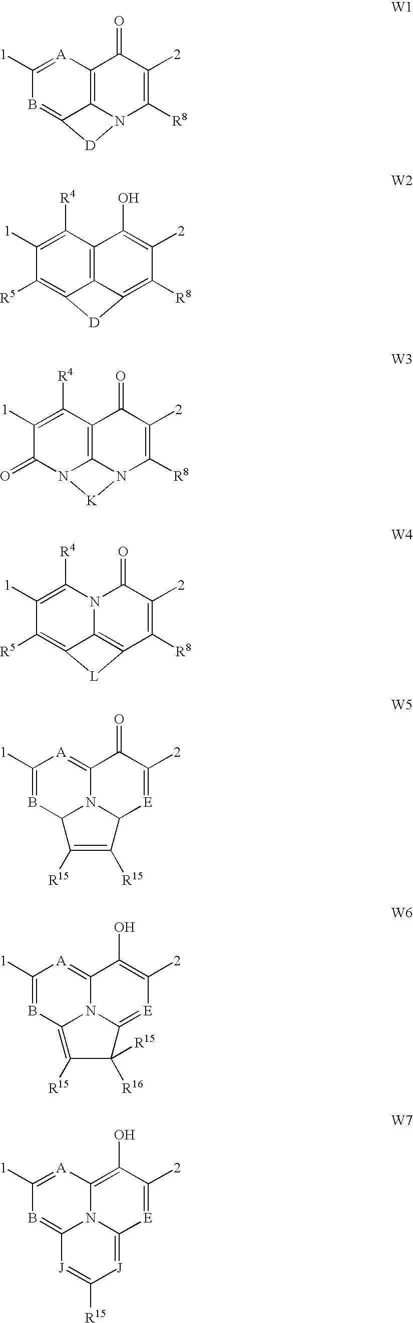 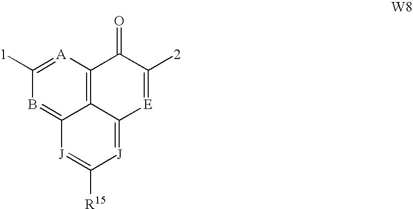
[It is intended that the R substituents and substituents A, B, D, E, J, K and L on the W^{XV-1} to W^{XV-8} Formulae above, as well as the W number under the formulas, be succeeded by the Roman Numeral XV.]
A^{XV} is CR^{XV-4} or nitrogen;
B^{XV} is CR^{XV-5} or nitrogen;
D^{XV} is
a) —(CR¹³R^{XV-14})ₐ^{XV}, where a^{XV} is 2 or 3
b) —(CR¹⁵R^{XV-16})₄—,
c) —Y^{XV}—CR¹³R^{XV-14}—CR¹³R^{XV-14}—,
d) —CR¹³R^{XV-14}—Y^{XV}—CR¹³R^{XV-14}—,
e) —Y^{XV}—CR¹³R^{XV-14}—Y^{XV}—,
f) —CR¹³R^{XV-14}—CR¹³R^{XV-14}—Y^{XV}—,
g) —Y^{XV}—(CR¹³R^{XV-16})ₙ^{XV}—,
h) —Y^{XV}—CR¹⁵═CR¹⁵—,
i) —CR¹⁵═CR¹⁵—Y^{XV}—,
k) —N═CR¹⁵—Y^{XV}—,
l) —(CR¹⁵R^{XV-16})_{b}^{XV}—N═CR¹⁵—, where b^{XV} is 0 or 1
m) —CR¹⁵═N—(CR¹⁵R^{XV-16})_{b}^{XV}—, where b^{XV} is 0 or 1
n) —N═N—,
o) —N═CR¹⁵—(CR¹⁵R¹⁶)_{b}^{XV}—, where b_{XV} is 0 or 1
p) —CR¹⁵═CR¹⁵—,
q) —N═N—Y^{XV},
r) —Y^{XV}N═N—,
s) —Y^{XV}—N═CR¹⁵, or
t) —CR¹⁵R^{XV-16}—Y^{XV}—CR¹⁵R^{XV-16}—CR¹⁵R^{XV-16}—;
E^{XV} is CR⁸ or nitrogen;
J^{XV} is CR¹⁵ or nitrogen;
K^{XV} is
a) —(CR¹⁵R^{XV-}¹⁶)ₐ^{XV}—, where a^{XV} is 2 or 3, or
b) —CR¹⁵═CR¹⁵—;
L^{XV} is
a) —(CR¹⁵R^{XV-16})ₐ^{XV}—, where a^{XV} is 2 or 3, or
b) —Y^{XV}—(CR¹⁵R^{XV-16})—(CR¹⁵R^{XV-16})—;
Y^{XV} is oxygen, S(O)ₘ^{XV}, or NR_{XV-7};
with the provisos that:
when W^{XV} is of formula W^{XV}1;
G^{XV is C}₁₋₄alkyl which is fully saturated and is substituted by hydroxy or morpholinyl, in which morpholinyl is attached through nitrogen;
A^{XV} is CR^{XV-4}; B^{XV} is CR^{XV-5}; and
R^{XV-8} is hydrogen then at least one of R^{XV-13}, R^{XV-14}, or R^{XV-7} is not hydrogen or C₁₋₇alkyl;
when W^{XV} is of formula W^{XV}1, A^{XV} is CR^{XV-4}, B^{XV} is CR^{XV-5}, D^{XV} is —Y^{XV}—CR¹³R^{XV-14}—CR¹³R^{XV-14}—, and R^{XV-8} is hydrogen then yXV is not oxygen;
when W^{XV} is of formula W^{XV}1, A^{XV} is CR^{XV-14}, and B^{XV} is CR^{XV-5} then D^{XV} is not —CR¹⁵═CR¹⁵—;
R^{XV-4} is H, halogen, or C₁₋₄alkyl optionally substituted by one to three halogens;
R^{XV-5} is
a) H,
b) halo,
c) OR^{XV-12},
d) SR^{XV-12},
e) C₁₋₇alkyl which may be partially unsaturated and optionally substituted by one or more substituents selected from OR^{XV-12}, SR^{XV-12}, NR^{XV-10}R^{XV-11}, or halo,
f) C₃₋₈cycloalkyl which may be partially unsaturated and is optionally substituted by one or more substituents selected from halogen, OR^{XV-12}, SR^{XV-12}, or NR^{XV-10}R^{XV-12},
g) (C═O)R^{XV-9},
h) S(O)ₘ^{XV}R^{XV-9},
i) (C═O)OR^{XV-2},
j) NHSO₂R^{XV-9},
k) nitro, or
l) cyano;
R^{XV-7} is
a) H,
b) C₁₋₇alkyl which may be partially unsaturated and optionally substituted by one or more substituents selected from OR^{XV-12}, SR^{XV-12}, NR^{XV-10}R^{XV-11}, or halo,
c) C₃₋₈cycloalkyl which may be partially unsaturated and is optionally substituted by one or more substituents selected from halogen, OR^{XV-12}, SR^{XV-12}, or NR^{XV-10}R^{XV-11},
d) aryl^{XV},
e) het^{XV},
f) (C═O)R^{XV-9}, or
g) S(O)ₘ^{XV}R^{XV-9};
R^{XV-8} is
a) H,
b) C₁₋₇alkyl which may be partially unsaturated and optionally substituted by one or more substituents selected from OR^{XV-12}, SR^{XV-12}, NR^{XV-10}R^{XV-11}, or halo,
c) OR^{XV-12}, or
d) SR^{XV-12};
R^{XV-9} is
a) C₁₋₇alkyl optionally substituted by OR^{XV-12} or NR^{XV-2}R^{XV-2},
b) C₃₋₈cycloalkyl optionally substituted by OR^{XV-12} or NR^{XV-2}R^{XV-2},
c) NR^{XV-10}R^{XV-11},
d) aryl^{XV}, or
e) het^{XV}, wherein said het^{XV} is bound through a carbon atom;
R^{XV-10} and R^{XV-11} are independently
a) H,
b) aryl^{XV},
c) C₁₋₇alkyl which may be partially unsaturated and is optionally substituted by one or more substituents selected from CONR²R^{XV-2}, CO₂R^{XV-2}, het^{XV}, aryl^{XV}, cyano, or halo,
d) C₂₋₇alkyl which may be partially unsaturated and is substituted by one or more substituents selected from NR^{XV-2}R^{XV-2}, OR^{XV-2}, or SR^{XV-2},
e) C₃₋₈cycloalkyl which may be partially unsaturated and is optionally substituted by one or more substituents selected from halogen, OR^{XV-2}, SR^{XV-2}, or NR^{XV-2}R^{XV-2}, or
f) R^{XV-10} and R^{XV-11} together with the nitrogen to which they are attached form a het^{XV};
R^{XV-12} is
a) H,
b) aryl^{XV},
c) hetXV,
d) C₁₋₇alkyl optionally substituted by arylXV, or halogen,
e) C₂₋₇alkyl substituted by OR^{XV-2}, SR^{XV-2}, or NR^{XV-2}R^{XV-2}, or
f) C₃₋₈cycloalkyl which may be partially unsaturated and is optionally substituted by one or more substituents selected from halogen, OR^{XV-2}, SR^{XV-2}, or NR^{XV-2}R^{XV-2};
R^{XV-13}, R^{XV-14}, R^{XV-15}, and R^{XV-16} are independently
a) H
b) C₁₋₇alkyl which may be partially unsaturated and optionally substituted by one or more OR^{XV-12}, SR^{XV-12}, NR^{XV-10}R^{XV-11}, or halo groups,
c) C₃₋₈cycloalkyl which may be partially unsaturated and is optionally substituted by one or more substituents selected from halogen, OR^{XV-12}, SR^{XV-12}, or NR^{XV-10}R^{XV-11},
d) aryl^{XV},
e) het^{XV}, wherein said het^{XV} is bound through a carbon atom,
f) OR^{XV-12},
g) SR^{XV-12},
h) NR^{XV-10}R^{XV-11};
i) (C═O)OR^{XV-2}, or
j) R¹³ and R¹⁴ or R¹⁵ and R¹⁶ together with the carbon to which they are attached form (C═O);
each m^{XV} is independently 0, 1 or 2;
each n^{XV} is independently 1 or 3;
aryl^{XV} is a phenyl radical or an ortho-fused bicyclic carbocyclic radical wherein at least one ring is aromatic, and aryl^{XV} may be optionally substituted with one or more substituents selected from halo, OH, cyano, NR^{XV-2}R^{XV-2}, CO₂R^{XV-2}, CF₃, C₁₋₆alkoxy, and C₁₋₆ alkyl which maybe further substituted by one to three SR^{XV-2}, NR^{XV-2}R^{XV-2}, OR^{XV-2}, or CO₂R^{XV-2} groups;
het^{XV} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and XVhet may be optionally substituted with one or more substituents selected from halo, OH, cyano, phenyl, CO₂R^{XV-2}, CF₃, C₁₋₆alkoxy, oxo, oxime, and C₁₋₆ alkyl which may be further substituted by one to three SR^{XV-2}, NR^{XV-2}R^{XV-2}, OR^{XV-2}, or CO₂R^{XV-2} groups;
halo or halogen is F, Cl, Br, I;
1 represents the point of attachment between W^{XV} and GXV;
2 represents the point of attachment between W^{XV} and the carbonyl group of Formula (I);
and pharmaceutically acceptable salts thereof.

2. A method of claim 1, wherein the compound administered has the Formula X 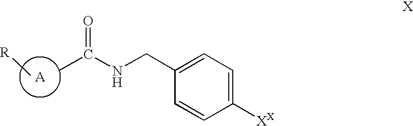
and pharmaceutically acceptable salts thereof; wherein ring A^{X} is a saturated or unsaturated fused double or triple heterocyclic ring having 1, 2, 3 or 4 heteroatoms selected from group consisting of oxygen, sulfur, or nitrogen; and wherein R^{X} and X^{X} are the appropriated substitutents, respectively.

3. The method of claim 2, wherein the compound administered is selected from the group consisting of compounds 1 to 16:

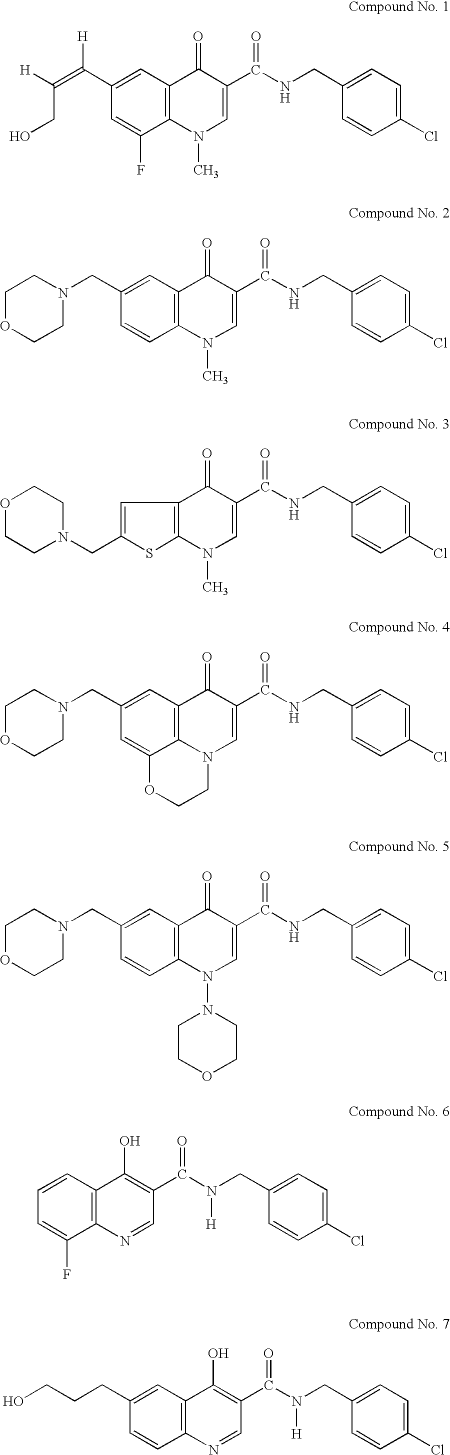

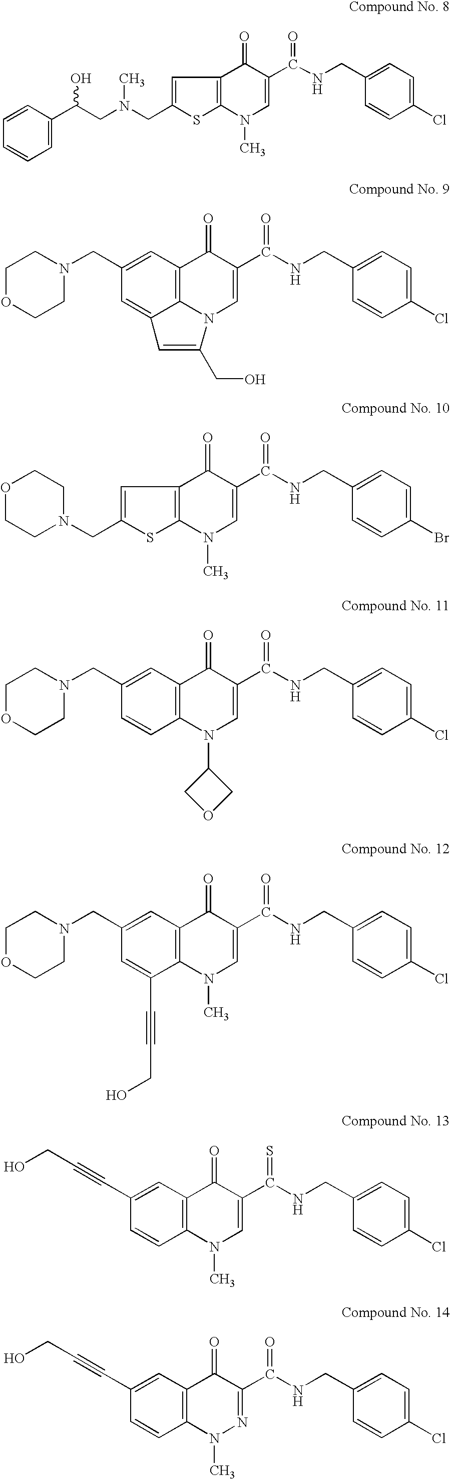

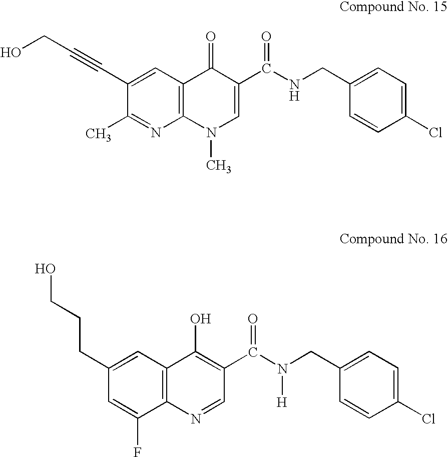

4. A method of claim 1, wherein the compound administered has the Formula XII 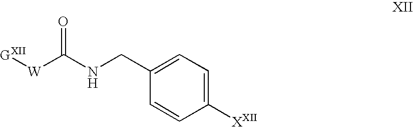
wherein,
X^{XII} is Cl, Br, F, CN or NO₂;
G^{XII} is
a) C₁₋₇alkyl which partially unsaturated and is substituted by hydroxy, or
b) C₁₋₄alkyl substituted by NR^{XII-1}R^{XII-2} or 4-tetrahydropyran;
R^{XII-1} is C₂₋₇alkyl substituted by hydroxy, C₁₋₄alkoxy, aryl^{XII}, or heteroaryl;
R^{XII-2} is hydrogen or C₁₋₇alkyl; or
R^{XII-1} and R^{XII-2} together with the nitrogen to which they are attached form morpholine which may be optionally substituted by ^{XII-}aryl or C₁₋₇alkyl;
W^{XII} is a heterocycle of formula W1^{XII}, W3^{XII}, or W4^{XII}; 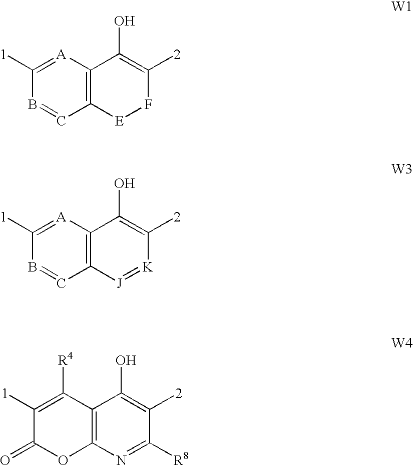
[It is intended that the R⁸ substituents and substituents A-F, J and K on the W1^{XII}, W3^{XII}, and W4^{XII} Formulae above, as well as the W number under the formulas, be succeeded by the Roman Numeral XII.]
A^{XII} is CR^{XII-4} or nitrogen;
B^{XII} is CR^{XII-5} or nitrogen;
C^{XII} is CR^{XII-6} or nitrogen;
E^{XII} and F^{XII} are such that
a) one is oxygen and the other is C(═O); or
b) E^{XII} is C(═O) and F^{XII} is NR^{XII-7};
J^{XII} and K^{XII} are such that
a) J^{XII} is nitrogen and K is CR^{XII-8}; or
b) J^{XII} is CR^{XII-6} and K is nitrogen;
with the provisos that when W^{XII} is of formula W3^{XII} and J^{XII} is nitrogen, then at least one of A^{XII} and B^{XII} is nitrogen;
R^{XII-4} is H, halogen, or C₁₋₄alkyl optionally substituted by one to three halogens;
R^{XII-5} is
a) H,
b) halo,
c) OR^{XII-12},
d) SR^{XII-12},
e) C₁₋₇alkyl which may be partially unsaturated and optionally substituted by one or more substituents selected from OR^{XII-12}, SR^{XII-12}, NR^{XII-10}R^{XII-11}, or halo,
f) C₃₋₈cycloalkyl which may be partially unsaturated and is optionally substituted by one or more substituents selected from halogen, OR^{XII-12}, SR^{XII-12}, or NR^{XIII-10}R^{XII-11},
g) (C═O)R^{XII-9},
h) S(O)ₘ^{XII}R^{XII-9},
i) (C═O)OR^{XII-2},
j) NHSO₂R^{XII-9},
k) nitro, or
l) cyano;
R^{XII-6} is
a) H,
b) halo,
c) aryl^{XII},
d) het^{XII},
e) OR^{XII-12},
f) SR^{XII-12},
g) C₁₋₇alkyl which may be partially unsaturated and optionally substituted by one or more substituents selected from OR^{XII-12}, SR^{XII-12}, NR^{XII-10}R^{XII-11}, aryl^{XII}, halo, C₃₋₈cycloalkyl optionally substituted by OR¹², or het^{XII} attached through a carbon atom,
h) NR^{XII-10}R^{XII-11},
i) C₃₋₈cycloalkyl which may be partially unsaturated and is optionally substituted by one or more substituents selected from halogen, OR^{XII-12}, SR^{XII-12}, or NR^{XII-10}R^{XII-11},
j) (C═O)R^{XII-9},
k) S(O)ₘ^{XII}R^{XII-9},
l) (C═O)OR^{XII-2},
m) NHSO₂R^{XII-9},
n) nitro, or
o) cyano;
R^{XII-7} is
a) H,
b) C₁₋₇alkyl which may be partially unsaturated and optionally substituted by one or more substituents selected from OR^{XII-12}, SR^{XII-12}, NR^{XII-10}R^{XII-11}, or halo,
c) C₃₋₈cycloalkyl which may be partially unsaturated and is optionally substituted by one or more substituents selected from halogen, OR^{XII-12}, SR^{XII-12}, or NR^{XII-10}R^{XII-11},
d) aryl^{XII}, or
e) het^{XII};
R⁸ is
a) H,
b) C₁₋₇alkyl which may be partially unsaturated and optionally substituted by one or more substituents selected from OR^{XII-12}, SR^{XII-12}, NR^{XII-10}R^{XII-11}, or halo,
c) OR^{XII-12}, or
d) SR^{XII-12};
R^{XII-9} is
a) C₁₋₇alkyl,
b) NR^{XII-10}R^{XII-11},
c) aryl^{XII}, or
d) het^{XII}, wherein said het^{XII} is bound through a carbon atom;
R^{XII-10} and R^{XII-11} are independently
a) H,
b) aryl^{XII},
c) C₁₋₇alkyl which may be partially unsaturated and is optionally substituted by one or more substituents selected from CONR²R², CO₂R², het^{XII}, aryl^{XII}, cyano, or halo,
d) C₂₋₇alkyl which may be partially unsaturated and is substituted by one or more substituents selected from NR^{XII-2}R^{XII-2}, OR^{XII-2}, or SR^{XII-2},
e) C₃₋₈cycloalkyl which may be partially unsaturated and is optionally substituted by one or more substituents selected from halogen, OR^{XII-2}, SR^{XII-2}, or NR^{XII-2}R^{XII-2}, or
f) R^{XII-10} and R^{XII-11} together with the nitrogen to which they are attached form a het^{XII};
R^{XII-12} is
a) H,
b) aryl^{XII},
c) het^{XII},
d) C₁₋₇alkyl optionally substituted by aryl^{XII}, het^{XII}, or halogen,
e) C₂₋₇alkyl substituted by OR^{XII-2}, SR^{XII-2}, or NR^{XII-2}R^{XII-2}, or
f) C₃₋₈cycloalkyl which may be partially unsaturated and is optionally substituted by one or more substituents selected from halogen, OR^{XII-2}, SR^{XII-2}, or NR^{XII-2}R^{XII-2};
each m^{XII} is independently 1 or 2;
aryl^{XII} is a phenyl radical or an ortho-fused bicyclic carbocyclic radical wherein at least one ring is aromatic, and aryl^{XII} maybe optionally substituted with one or more substituents selected from halo, OH, cyano, NR^{XII-2}R^{XII-2}, CO₂R^{XII-2}, CF₃, C₁₋₆alkoxy, and C₁₋₆ alkyl which maybe further substituted by one to three SR^{XII-2} NR^{XII-2}R^{XII-2}, OR^{XII-2}, or CO₂R^{XII-2} groups;
het^{XII} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XII} may be optionally substituted with one or more substituents selected from halo, OH, cyano, phenyl, CO₂R^{XII-2}, CF₃, C₁₋₆alkoxy, oxo, oxime, and C₁₋₆ alkyl which may be further substituted by one to three SR^{XII-2}, NR^{XII-2}R^{XII-2}, OR^{XII-2}, or CO₂R^{XII-2} groups;
halo or halogen is F, Cl, Br, I;
1 represents the point of attachment between W^{XII} and G^{XII};
2 represents the point of attachment between W^{XII} and the carbonyl group of Formula (I);
and a pharmaceutically acceptable salt thereof.

5. The method according to claim 4, wherein the compound administered is:
(1) N-(4-chlorobenzyl)-4-hydroxy-6-(4-morpholinylmethyl)-2-oxo-2H-pyrano[2,3-c]pyridine-3-carboxamide;
(2) N-(4-chlorobenzyl)-4-hydroxy-6-(3-hydroxy-1-propynyl)-1-oxo-1H-isochromene-3-carboxamide;
(3) N-(4-chlorobenzyl)-4-hydroxy-1-oxo-6-(tetrahydro-2H-pyran-4-ylmethyl)-1H-isochromene-3-carboxamide;
(4) N-(4-chlorobenzyl)-4-hydroxy-6-(4-morpholinylmethyl)-1-oxo-1H-isochromene-3-carboxamide;
(5) N-(4-chlorobenzyl)-5-hydroxy-3-(3-hydroxy-1-propynyl)-8-oxo-7,8-dihydro[1,7]naphthyridine-6-carboxamide;
(6) N-(4-chlorobenzyl)-4-hydroxy-6-(3-hydroxy-1-propynyl)-1-oxo-1,2-dihydro-3-isoquinolinecarboxamide;
(7) N-(4-chlorobenzyl)-4-hydroxy-6-(4-morpholinylmethyl)-1-oxo-1,2-dihydro-3-isoquinolinecarboxamide;
(8) N-(4-chlorobenzyl)-4-hydroxy-6-(3-hydroxy-1-propynyl)[1,7]naphthyridine-3-carboxamide;
(9) N-(4-chlorobenzyl)-8-ethoxy-4-hydroxy-6-(3-hydroxy-1-propynyl)[1,7]naphthyridine-3-carboxamide;
(10) N-(4-chlorobenzyl)-4-hydroxy-6-(4-morpholinylmethyl)[1,7]naphthyridine-3-carboxamide;
(11) N-(4-chlorobenzyl)-8-ethoxy-4-hydroxy-6-(4-morpholinylmethyl)[1,7]naphthyridine-3-carboxamide;
(12) N-(4-chlorobenzyl)-4-hydroxy-6-(3-hydroxy-1-propynyl)[1,5]naphthyridine-3-carboxamide;
(13) N-(4-chlorobenzyl)-4-hydroxy-6-(4-morpholinylmethyl)[1,5]naphthyridine-3-carboxamide;
(14) N-(4-chlorobenzyl)-4-hydroxy-6-(tetrahydro-2H-pyran-4-ylmethyl)[1,5]naphthyridine-3-carboxamide;
(15) N-(4-chlorobenzyl)-8-hydroxy-2-(3-hydroxy-1-propynyl)pyrido[3,2-d]pyrimidine-7-carboxamide;
(16) N-(4-chlorobenzyl)-8-hydroxy-2-(4-morpholinylmethyl)pyrido[3,2-d]pyrimidine-7-carboxamide;
(17) N-(4-chlorobenzyl)-5-hydroxy-3-(4-morpholinylmethyl)[1,7]naphthyridine-6-carboxamide;
(18) N-(4-chlorobenzyl)-5-hydroxy-3-(3-hydroxy-1-propynyl)[1,7]naphthyridine-6-carboxamide;
(19) N-(4-chlorobenzyl)-5-hydroxy-3-(tetrahydro-2H-pyran-4-ylmethyl)[1,7]naphthyridine-6-carboxamide;
(20) N-(4-chlorobenzyl)-4-hydroxy-6-(4-morpholinylmethyl)-3-isoquinolinecarboxamide;
(21) N-(4-chlorobenzyl)-4-hydroxy-6-(3-hydroxy-1-propynyl)-3-isoquinolinecarboxamide;
(22) N-(4-chlorobenzyl)-4-hydroxy-6-(tetrahydro-2H-pyran-4-ylmethyl)-3-isoquinolinecarboxamide; or
a pharmaceutically acceptable salt thereof.

6. The method of claim 1, wherein the compound administered has the Formula XIII 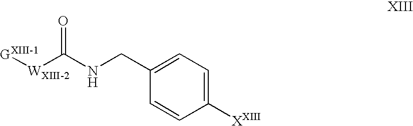
wherein,
X^{XIII} is Cl, Br, F, CN or NO₂;
G^{XIII} is
a) C₃₋₇alkyl which is partially unsaturated and is substituted by hydroxy, or
b) C₁₋₇alkyl substituted by NR^{XIII-1}R² or 4-tetrahydropyran;
R^{XIII-1} is C₂₋₇alkyl substituted by hydroxy, C₁₋₄alkoxy, aryl^{XIII}, or heteroaryl;
R^{XIII-2} is hydrogen or C₁₋₇alkyl; or
R^{XIII-1} and R^{XIII-2} together with the nitrogen to which they are attached form morpholine which may be optionally substituted by aryl or C₁₋₇alkyl;
W^{XIII} is 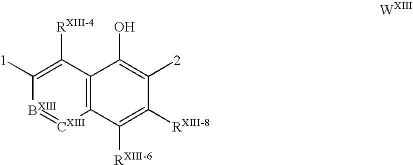
B^{XIII} is C^{RXIII-5} or nitrogen;
C^{XIII} is C^{RXIII-6} or nitrogen;
with the provisos that B^{XIII} and C^{XIII} cannot be both nitrogen;
R^{XIII-4} is H, halogen, or C₁₋₄alkyl optionally substituted by one to three halogens;
R^{XIII-5} is
a) H,
b) halo,
c)OR^{XIII-12},
d) SR^{XIII-12},
e) C₁₋₇alkyl which may be partially unsaturated and optionally substituted by one or more substituents selected from OR^{XIII-12}, SR^{XIII-12}, NR^{XIII-10}R^{XIII-11}, or halo,
f) C₃₋₈cycloalkyl which may be partially unsaturated and is optionally substituted by one or more substituents selected from halogen, OR^{XIII-12}, SR^{XIII-2}, or NR^{XIII-10}R^{XIII-11},
g) (C═O)R^{XIII-9},
h) S(O)ₘ^{XIII}R^{XIII-9},
i) (C═O)OR^{XIII-2},
j) NHSO₂R^{XIII-9},
k) nitro, or
l) cyano;
R^{XIII-6} is
a) H,
b) halo,
c) aryl^{XIII},
d) het^{XIII}, or
e) R^{XIII-7};
R^{XIII-7} is
a) OR^{XIII-12},
b) SR^{XIII-12},
c) C₁₋₇alkyl which may be partially unsaturated and optionally substituted by one or more substituents selected from OR^{XIII-12}, SR^{XIII-12}, NR^{XIII-10}R^{XIII-11}, aryl^{XIII}, halo, C₃₋₈cycloalkyl optionally substituted by OR^{XIII-12}, or het^{XIII} attached through a carbon atom,
d) NR^{XIII-10}R^{XIII-11},
e) C₃₋₈cycloalkyl which may be partially unsaturated and is optionally substituted by one or more substituents selected from halogen, OR^{XIII-12}, SR^{XIII-12}, or NR^{XIII-10}R^{XIII-11},
f) (C═O)R^{XIII-9},
g) S(O)ₘ^{XIII}R^{XIII-9},
h) (C═O)OR^{XIII-2},
i) NHSO₂R^{XIII-9},
j) nitro, or
k) cyano;
R^{XIII-8} is
a) H,
b) C₁₋₇alkyl which may be partially unsaturated and optionally substituted by one or more substituents selected from OR^{XIII-12}, SR^{XIII-12}, NR^{XIII-10}R^{XIII-11}, or halo,
c) OR^{XII-12}, or
d) SR^{XIII-12};
R^{XIII-9} is
a) C₁₋₇alkyl,
b) NR^{XIII-10}R^{XIII-11},
c) aryl^{XIII}, or
d) het^{XIII}, wherein said het^{XIII} is bound through a carbon atom;
R^{XIII-10} and R^{XIII-11} are independently
a) H,
b) aryl^{XIII},
c) C₁₋₇alkyl which may be partially unsaturated and is optionally substituted by one or more substituents selected from CONR^{XIII-2}R^{XIII-2}, CO₂R^{XIII-2}, het^{XIII}, aryl^{XIII}, cyano, or halo,
d) C₂₋₇alkyl which may be partially unsaturated and is substituted by one or more substituents selected from NR^{XIII-2}R^{XIII-2}, OR^{XIII-2}, or SR^{XIII-2},
e) C₃₋₈cycloalkyl which may be partially unsaturated and is optionally substituted by one or more substituents selected from halogen, OR^{XIII-2}, SR^{XIII-2}, or NR^{XIII-2}R^{XIII-2}, or
f) R^{XIII-10} and R^{XIII-11} together with the nitrogen to which they are attached form a het^{XIII};
R^{XIII-12} is
a) H,
b) aryl^{XIII},
c) het^{XIII},
d) C₁₋₇alkyl optionally substituted by aryl^{XIII}, het^{XIII}, or halogen,
e) C₂₋₇alkyl substituted by OR^{XIII-2}, SR^{XIII-2}, or NR^{XIII-2}R^{XIII-2}, or
f) C₃₋₈cycloalkyl which may be partially unsaturated and is optionally substituted by one or more substituents selected from halogen, OR^{XIII-2}, SR^{XIII-2}, or NR^{XIII-2}R^{XIII-2};
each m^{XIII} is independently 1 or 2;
aryl^{XIII} is a phenyl radical or an ortho-fused bicyclic carbocyclic radical wherein at least one ring is aromatic, and aryl^{XII} maybe optionally substituted with one or more substituents selected from halo, OH, cyano, NR^{XIII-2}R^{XIII-2}, CO₂R^{XIII-2}, CF₃, C₁₋₆alkoxy, and C₁₋₆ alkyl which maybe further substituted by one to three SR^{XIII-2}, NR^{XIII-2}R^{XIII-2}, OR^{XIII-2}, or CO₂R^{XIII-2} groups;
het^{XIII} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XIII} may be optionally substituted with one or more substituents selected from halo, OH, cyano, phenyl, CO₂R^{XIII-2}, CF₃, C₁₋₆alkoxy, oxo, oxime, and C₁₋₆ alkyl which may be further substituted by one to three SR^{XIII-2}, NR^{XIII-2}R^{XIII-2}, OR^{XIII-2}, or CO₂R^{XIII-2} groups;
halo or halogen is F, Cl, Br, I;
1 represents the point of attachment between W^{XIII} and G^{XIII};
2 represents the point of attachment between W^{XIII} and the carbonyl group of Formula (XIV);
and a pharmaceutically acceptable salt thereof.

7. The method of claim 6, wherein W^{XIII-2} is of the formula W2.1, as set forth on page 10 of the specification of application Ser. No. 09/887,226 filed Jun. 22, 2001.

8. The method of claim 6, wherein W^{XIII-2} is of the formula W2.2, as set forth on page 10 of the specification of application Ser. No. 09/887,226, filed Jun. 22, 2001.

9. The method of claim 6, wherein the compound administered is selected from the group consisting of
(1) N-(4-chlorobenzyl)-5-hydroxy-3-(4-morpholinylmethyl)-6-isoquinolinecarboxamide;
(2) N-(4-chlorobenzyl)-5-hydroxy-3-(tetrahydro-2H-pyran-4-ylmethyl)-6-isoquinolinecarboxamide;
(3) N-(4-chlorobenzyl)-5-hydroxy-3-(3-hydroxy-1-propynyl)-6-isoquinolinecarboxamide;
(4) N-(4-chlorobenzyl)-5-hydroxy-3-(4-morpholinylmethyl)-6-quinolinecarboxamide;
or a pharmaceutically acceptable salt thereof.

10. The method of claim 1, wherein the compound administered has the Formula XIV 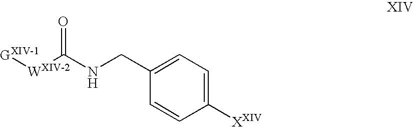
wherein,
X^{XIV} is Cl, Br, F, CN, or NO₂;
G^{XIV} is
a) C₁₋₄alkyl which is fully saturated or partially unsaturated and is substituted by hydroxy, or
b) C₁₋₄alkyl substituted by NR^{XIV-1}R^{XIV-2} or 4-tetrahydropyran;
R^{XIV-1} is C₂₋₇alkyl substituted by hydroxy, C₁₋₄alkoxy, heteroaryl, or aryl^{XIV};
R^{XIV-2} is hydrogen or C₁₋₇alkyl; or
R^{XIV-1} and R^{XIV-2} together with the nitrogen to which they are attached form morpholine which may be optionally substituted by aryl^{XIV} or C₁₋₇alkyl; or pyrrolidine substituted by hydroxy;
W^{XIV} is a heterocycle of formula W1^{XIV}, W2^{XIV}, W3^{XIV}, W4^{XIV}, W5^{XIV}, W6^{XIV}, W7^{XIV}, W8^{XIV}, W9^{XIV}, W10^{XIV}, W11^{XIV}, W12^{XIV}, W13^{XIV}, W14^{XIV}, W15^{XIV}, W16^{XIV}, W17^{XIV}, W18^{XIV}, W19^{XIV}, W20^{XIV}, W21^{XIV} or W22^{XIV} 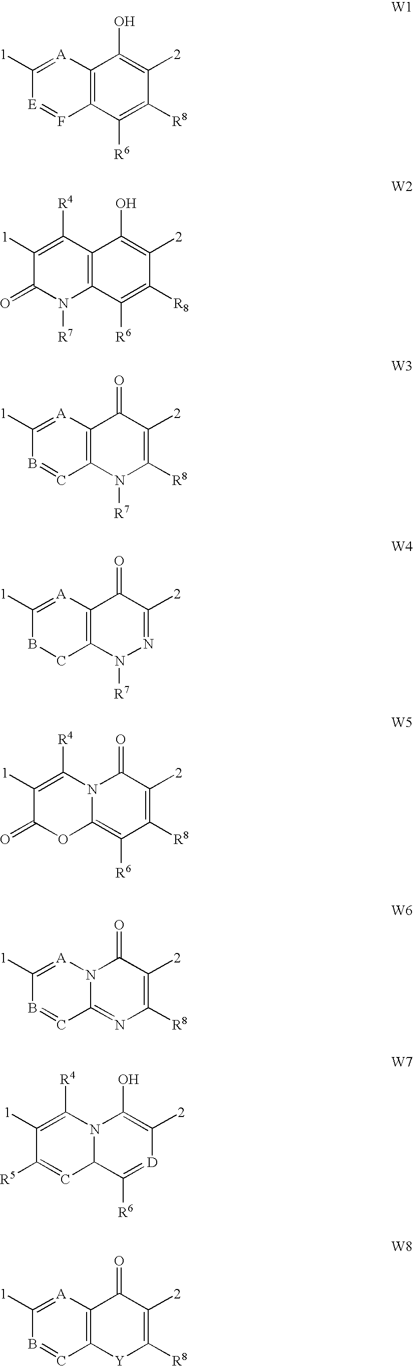 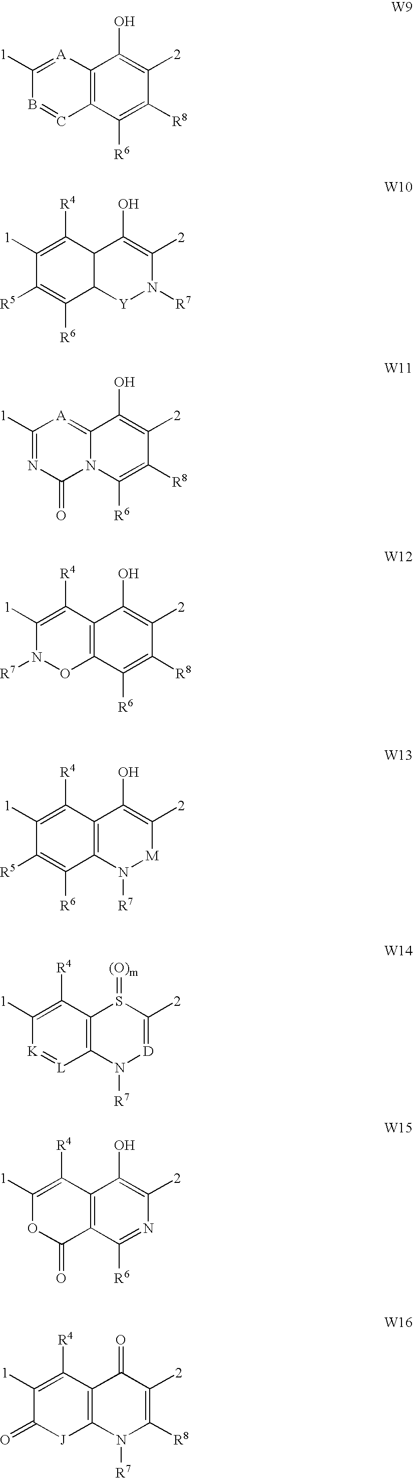 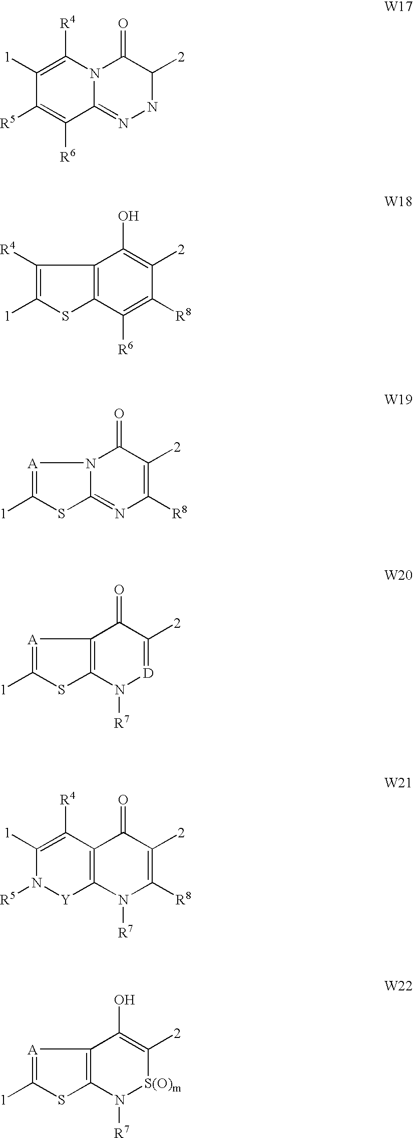
[It is intended that the R substituents and substituents A-F, J, K, L, M and Y on the W Formulae above, as well as the W number under the formulas, be succeeded by the Roman Numeral XIV.]
A^{XIV} is CR^{XV-4} or nitrogen;
B^{XIV} is CR^{XV-5} or nitrogen;
C^{XIV} is CR^{XV-6} or nitrogen;
D^{XIV} is CR^{XV-8} or nitrogen;
E^{XIV} and F are such that one is oxygen and the other is C(═O);
J^{XIV} is NR^{XV-7} or oxygen;
K^{XIV} and L^{XIV} are defined such that
a) K^{XIV} is CR^{XIV-5} and L^{XIV} is CR^{XV-6}, or
b) K^{XIV} is absent and L^{XIV} is sulfur;
M^{XIV} is oxygen, sulfur, or S(O)ₘ;
Y^{XIV} is oxygen or sulfur;
The suffixed numbered “R” groups in the definitions thereof correspond to the numbered groups of Formulas W1-W22.
R^{XIV-4} is H, halogen, or C₁₋₄alkyl optionally substituted by one to three halogens;
R^{XIV-5} is
a) H,
b) halo,
c) OR^{XIV-12},
d) SR^{XIV-12},
e) C₁₋₇alkyl which may be partially unsaturated and optionally substituted by one or more substituents selected from OR^{XIV-12}, SR^{XIV-12}, NR^{XIV-10}R^{XIV-11}, or halo,
f) C₃₋₈cycloalkyl which may be partially unsaturated and is optionally substituted by one or more substituents selected from halogen, OR^{1XIV-2}, SR^{XIV-12}, or NR^{XIV-10}R^{XIV-11},
g) (C═O)R^{XIV-9},
h) S(O)ₘ^{XIV}R^{XIV-9},
i) (C═O)OR^{XIV-2},
j) NHSO₂R^{XIV-9},
k) nitro, or
l) cyano;
R^{XIV-6} is
a) H,
b) halo,
c) aryl^{XIV},
d) het^{XIV},
e) OR^{XIV-12},
f) SR^{XIV-12},
g) C₁₋₇alkyl which may be partially unsaturated and optionally substituted by one or more substituents selected from OR^{XIV-12}, SR^{XIV-12}, NR^{XIV-10}R^{XIV-11}, aryl^{XIV}, halo, C₃₋₈cycloalkyl optionally substituted by OR^{XIV-12}, or het^{XIV} attached through a carbon atom,
h) NR^{XIV-10}R^{XIV-11},
i) C₃₋₈cycloalkyl which may be partially unsaturated and is optionally substituted by one or more substituents selected from halogen, OR^{XIV-12}, SR^{XIV-12}, or NR^{XIV-10}R^{XIV-11},
j) (C═O)R^{XIV-9},
k) S(O)ₘ^{XIV}R^{XIV-9},
l) (C═O)OR^{XIV-2},
m) NHSO₂R^{XIV-9},
n) nitro, or
o) cyano;
R^{XV-7} is
a) H,
b) C₁₋₇alkyl which may be partially unsaturated and optionally substituted by one or more substituents selected from OR^{XIV-12}, SR^{XIV-12}, NR^{XIV-10}R^{XIV-11}, or halo,
c) C₃₋₈cycloalkyl which may be partially unsaturated and is optionally substituted by one or more substituents selected from halogen, OR^{XIV-12}, SR^{XIV-12}, or NR^{XIV-10}R^{XIV-11},
d) aryl^{XIV}, or
e) het^{XIV};
R^{XIV-8} is
a) H,
b) C₁₋₇alkyl which may be partially unsaturated and optionally substituted by one or more substituents selected from OR^{XIV-12}, SR^{XIV-12}, NR^{XIV-10}R^{XIV-11}, or halo,
c) OR^{XIV-12}, or
d) SR^{XIV-12};
R^{XIV-9} is
a) C₁₋₇alkyl,
b) NR^{XIV-10}R^{XIV-11},
c) aryl^{XIV}, or
d) het^{XIV}, wherein said hetXV is bound through a carbon atom;
R^{XIV-10} and R^{XIV-11} are independently
a) H,
b) aryl^{XIV},
c) C₁₋₇alkyl which may be partially unsaturated and is optionally substituted by one or more substituents selected from CONR^{XIV-2}R^{XIV-2}, CO₂R^{XIV-2}, het^{XIV}, aryl^{XIV}, cyano, or halo,
d) C₂₋₇alkyl which may be partially unsaturated and is substituted by one or more substituents selected from NR^{XIV-2}R^{XIV-2}, OR^{XIV-2}, or SR^{XIV-2},
e) C₃₋₈cycloalkyl which may be partially unsaturated and is optionally substituted by one or more substituents selected from halogen, OR^{XIV-2}, SR^{XIV-2}, or NR^{XIV-2}R^{XIV-2}, or
f) R^{XIV-10} and R^{XIV-11} together with the nitrogen to which they are attached form a het^{XIV};
R^{XIV-12} is
a) H,
b) aryl^{XIV},
c) het^{XIV},
d) C₁₋₇alkyl optionally substituted by aryl^{XIV}, or halogen,
e) C₂₋₇alkyl substituted by OR^{XIV-2}, SR^{XIV-2}, or NR^{XIV-2}R^{XIV-2}, or
f) C₃₋₈cycloalkyl which may be partially unsaturated and is optionally substituted by one or more substituents selected from halogen, OR^{XIV-2}, SR^{XIV-2}, or NR^{XIV-2}R^{XIV-2};
each m^{XIV} is independently 1 or 2;
aryl^{XIV} is a phenyl radical or an ortho-fused bicyclic carbocyclic radical wherein at least one ring is aromatic, and aryl^{XIV} may be optionally substituted with one or more substituents selected from halo, OH, cyano, NR^{XIV-2}R^{XIV-2}, CO₂R^{XIV-2}, CF₃, C₁₋₆alkoxy, and C₁₋₆ alkyl which maybe further substituted by one to three SR^{XIV-2}, NR^{XIV-2}R^{XIV-2}, OR^{XIV-2}, or CO₂R^{XIV-2}, groups;
het^{XIV} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XIV} may be optionally substituted with one or more substituents selected from halo, OH, cyano, phenyl, CO₂R^{XIV-2}, CF₃, C₁₋₆alkoxy, oxo, oxime, and C₁₋₆ alkyl which may be further substituted by one to three SR^{XIV-2}, NR^{XIV-2}R^{XIV-2}, OR^{XIV-2}, or CO₂R^{XIV-2} groups;
halo or halogen is F, Cl, Br, I;
1 represents the point of attachment between W^{XIV} and G^{XIV};
2 represents the point of attachment between W^{XIV} and the carbonyl group of Formula (I);
or a pharmaceutically acceptable salt thereof.

11. The method according to claim 10, wherein the compound administered is:
(1) N-(4-chlorobenzyl)-5-hydroxy-3-(3-hydroxy-1-propynyl)-2-oxo-2H-chromene-6-carboxamide;
(2) N-(4-chlorobenzyl)-5-hydroxy-3-(3-hydroxypropyl)-2-oxo-2H-chromene-6-carboxamide;
(3) N-(4-chlorobenzyl)-5-hydroxy-3-(4-morpholinylmethyl)-2-oxo-2H-chromene-6-carboxamide;
(4) N-(4-chlorobenzyl)-5-hydroxy-4-methyl-3,8-bis(4-morpholinylmethyl)-2-oxo-2H-chromene-6-carboxamide;
(5) N-(4-chlorobenzyl)-5-hydroxy-3-(3-hydroxy-1-propynyl)-1-methyl-2-oxo-1,2-dihydro-6-quinolinecarboxamide;
(6) N-(4-chlorobenzyl)-5-hydroxy-3-(3-hydroxypropyl)-1-methyl-2-oxo-1,2-dihydro-6-quinolinecarboxamide;
(7) N-(4-chlorobenzyl)-5-hydroxy-1-methyl-3-(4-morpholinylmethyl)-2-oxo-1,2-dihydro-6-quinolinecarboxamide;
(8) N-(4-chlorobenzyl)-6-(3-hydroxy-1-propynyl)-1-methyl-4-oxo-1,4-dihydro[1,7]naphthyridine-3-carboxamide;
(9) N-(4-chlorobenzyl)-6-(3-hydroxypropyl)-1-methyl-4-oxo-1,4-dihydro[1,7]naphthyridine-3-carboxamide;
(10) N-(4-chlorobenzyl)-1-methyl-6,8-bis(morpholin-4-ylmethyl)-4-oxo-1,4-dihydro-1,7-naphthyridine-3-carboxamide;
(11) N-(4-chlorobenzyl)-8-ethoxy-6-(3-hydroxy-1-propynyl)-1-methyl-4-oxo-1,4-dihydro[1,7]naphthyridine-3-carboxamide;
(12) N-(4-chlorobenzyl)-8-ethoxy-6-(3-hydroxypropyl)-1-methyl-4-oxo-1,4-dihydro[1,7]naphthyridine-3-carboxamide;
(13) N-(4-chlorobenzyl)-1-methyl-6-(4-morpholinylmethyl)-4-oxo-1,4-dihydro[1,7]naphthyridine-3-carboxamide;
(14) 8-chloro-N-(4-chlorobenzyl)-1-methyl-6-(4-morpholinylmethyl)-4-oxo-1,4-dihydro1,7]naphthyridine-3-carboxamide;
(15) N-(4-chlorobenzyl)-8-ethoxy-1-methyl-6-(4-morpholinylmethyl)-4-oxo-1,4-dihydro[1,7]naphthyridine-3-carboxamide;
(16) N-(4-chlorobenzyl)-6-(3-hydroxypropyl)-1-methyl-4-oxo-1,4-dihydro[1,5]naphthyridine-3-carboxamide;
(17) N-(4-chlorobenzyl)-6-(3-hydroxy-1-propynyl)-1-methyl-4-oxo-1,4-dihydro[1,5]naphthyridine-3-carboxamide;
(18) N-(4-chlorobenzyl)-1-methyl-6-(4-morpholinylmethyl)-4-oxo-1,4-dihydro[1,5]naphthyridine-3-carboxamide;
(19) N-(4-chlorobenzyl)-1-methyl-4-oxo-6-(tetrahydro-2H-pyran-4-ylmethyl)-1,4-dihydro[1,5]naphthyridine-3-carboxamide;
(20) N-(4-chlorobenzyl)-8-ethyl-3-(4-morpholinylmethyl)-5-oxo-5,8-dihydropyrido[2,3-c]pyridazine-6-carboxamide;
(21) N-(4-chlorobenzyl)-2-(3-hydroxypropyl)-5-methyl-8-oxo-5,8-dihydropyrido[3,2-d]pyrimidine-7-carboxamide;
(22) N-(4-chlorobenzyl)-2-(3-hydroxy-1-propynyl)-5-methyl-8-oxo-5,8-dihydropyrido[3,2-d]pyrimidine-7-carboxamide;
(23) N-(4-chlorobenzyl)-5-methyl-2-(4-morpholinylmethyl)-8-oxo-5,8-dihydropyrido[3,2-d]pyrimidine-7-carboxamide;
(24) N-(4-chlorobenzyl)-6-(3-hydroxy-1-propynyl)-1-methyl-4-oxo-1,4-dihydropyrido[2,3-c]pyridazine-3-carboxamide;
(25) N-(4-chlorobenzyl)-6-(3-hydroxypropyl)-1-methyl-4-oxo-1,4-dihydropyrido[2,3-c]pyridazine-3-carboxamide;
(26) N-(4-chlorobenzyl)-1-methyl-6-(4-morpholinylmethyl)-4-oxo-1,4-dihydropyrido[2,3-c]pyridazine-3-carboxamide;
(27) N-(4-chlorobenzyl)-1-methyl-4-oxo-6-(tetrahydro-2H-pyran-4-ylmethyl)-1,4-dihydropyrido[2,3-c]pyridazine-3-carboxamide;
(28) N-(4-chlorobenzyl)-6-(3-hydroxypropyl)-4-oxo-1,4-dihydropyrido[3,4-c]pyridazine-3-carboxamide;
(29) N-(4-chlorobenzyl)-6-(3-hydroxy-1-propynyl)-4-oxo-1,4-dihydropyrido[3,4-c]pyridazine-3-carboxamide;
(30) N-(4-chlorobenzyl)-6-(3-hydroxypropyl)-1-methyl-4-oxo-1,4-dihydropyrido[3,4-c]pyridazine-3-carboxamide;
(31) N-(4-chlorobenzyl)-6-(3-hydroxy-1-propynyl)-1-methyl-4-oxo-1,4-dihydropyrido[3,4-c]pyridazine-3-carboxamide;
(32) N-(4-chlorobenzyl)-1-methyl-6-(4-morpholinylmethyl)-4-oxo-1,4-dihydropyrido[3,4-c]pyridazine-3-carboxamide;
(33) N-(4-chlorobenzyl)-6-(4-morpholinylmethyl)-4-oxo-1,4-dihydropyrido[3,4-c]pyridazine-3-carboxamide;
(34) N-(4-chlorobenzyl)-7-(3-hydroxy-1-propynyl)-4-oxo-4H-pyrido[1,2-a]pyrimidine-3-carboxamide;
(35) N-(4-chlorobenzyl)-7-(3-hydroxypropyl)-4-oxo-4H-pyrido[1,2-a]pyrimidine-3-carboxamide;
(36) N-(4-chlorobenzyl)-7-(4-morpholinylmethyl)-4-oxo-4H-pyrido[1,2-a]pyrimidine-3-carboxamide;
(37) N-(4-chlorobenzyl)-4-hydroxy-7-(3-hydroxy-1-propynyl)-2-oxo-2H-pyrido[1,2-a]pyrimidine-3-carboxamide;
(38) N-(4-chlorobenzyl)-2-hydroxy-7-(3-hydroxypropyl)-4-oxo-4H-pyrido[1,2-a]pyrimidine-3-carboxamide;
(39) N-(4-chlorobenzyl)-7-(3-hydroxypropyl)-4-oxo-4H-pyrazino[1,2-a]pyrimidine-3-carboxamide;
(40) N-(4-chlorobenzyl)-7-(3-hydroxy-1-propynyl)-4-oxo-4H-pyrazino[1,2-a]pyrimidine-3-carboxamide;
(41) N-(4-chlorobenzyl)-7-(4-morpholinylmethyl)-4-oxo-4H-pyrazino[1,2-a]pyrimidine-3-carboxamide;
(42) N-(4-chlorobenzyl)-7-(3-hydroxy-1-propynyl)-4-oxo-4H-pyrimido[1,2-b]pyridazine-3-carboxamide;
(43) N-(4-chlorobenzyl)-7-(3-hydroxypropyl)-4-oxo-4H-pyrimido[1,2-b]pyridazine-3-carboxamide;
(44) N-(4-chlorobenzyl)-4-oxo-7-(tetrahydro-2H-pyran-4-ylmethyl)-4H-pyrimido[1,2-b]pyridazine-3-carboxamide;
(45) N-(4-chlorobenzyl)-7-(4-morpholinylmethyl)-4-oxo-4H-pyrimido[1,2-b]pyridazine-3-carboxamide;
(46) N-(4-chlorobenzyl)-7-(3-hydroxypropyl)-4-oxo-4H-pyrimido[1,2-a]pyrimidine-3-carboxamide;
(47) N-(4-chlorobenzyl)-7-(3-hydroxy-1-propynyl)-4-oxo-4H-pyrimido[1,2-a]pyrimidine-3-carboxamide;
(48) N-(4-chlorobenzyl)-4-oxo-7-(tetrahydro-2H-pyran-4-ylmethyl)-4H-pyrimido[1,2-a]pyrimidine-3-carboxamide;
(49) N-(4-chlorobenzyl)-7-(4-morpholinylmethyl)-4-oxo-4H-pyrimido[1,2-a]pyrimidine-3-carboxamide;
(50) N-(4-chlorobenzyl)-2-(3-hydroxypropyl)-8-oxo-8H-pyrimido[1,2-b][1,2,4]triazine-7-carboxamide;
(51) NA-(4-chlorobenzyl)-2-(3-hydroxy-1-propynyl)-8-oxo-8H-pyrimido[1,2-b][1,2,4]triazine-7-carboxamide;
(52) N-(4-chlorobenzyl)-7-(4-morpholinylmethyl)-4-oxo-4H-quinolizine-3-carboxamide;
(53) N-(4-chlorobenzyl)-7-(3-hydroxypropyl)-4-oxo-4H-quinolizine-3-carboxamide;
(54) N-(4-chlorobenzyl)-7-(3-hydroxy-1-propynyl)-4-oxo-4H-quinolizine-3-carboxamide;
(55) N-(4-chlorobenzyl)-7-(3-hydroxy-1-propynyl)-4-oxo-4H-pyrido[1,2-a]pyrazine-3-carboxamide;
(56) N-(4-chlorobenzyl)-7-(3-hydroxypropyl)-4-oxo-4H-pyrido[1,2-a]pyrazine-3-carboxamide;
(57) N-(4-chlorobenzyl)-7-(4-morpholinylmethyl)-4-oxo-4H-pyrido[1,2-a]pyrazine-3-carboxamide;
(58) N-(4-chlorobenzyl)-3-(4-morpholinylmethyl)-6-oxo-6H-pyrido[1,2-a]pyrimidine-7-carboxamide;
(59) N-(4-chlorobenzyl)-6-(4-morpholinylmethyl)-4-oxo-4H-chromene-3-carboxamide;
(60) N-(4-chlorobenzyl)-6-(3-hydroxy-1-propynyl)-4-oxo-4H-chromene-3-carboxamide;
(61) N-(4-chlorobenzyl)-6-(((3R)-3-hydroxypyrrolidinyl)methyl)-4-oxo-4H-chromene-3-carboxamide;
(62) N-(4-chlorobenzyl)-6,8-bis(4-morpholinylmethyl)-4-oxo-4H-chromene-3-carboxamide;
(63) N-(4-chlorobenzyl)-6-(3-hydroxypropyl)-4-oxo-4H-chromene-3-carboxamide;
(64) N-(4-chlorobenzyl)-6-(3-hydroxypropyl)-4-oxo-4H-pyrano[2,3-b]pyridine-3-carboxamide;
(65) N-(4-chlorobenzyl)-6-(3-hydroxy-1-propynyl)-4-oxo-4H-pyrano[2,3-b]pyridine-3-carboxamide;
(66) N-(4-chlorobenzyl)-4-oxo-6-(tetrahydro-2H-pyran-4-ylmethyl)-4H-pyrano[2,3-b]pyridine-3-carboxamide;
(67) N-(4-chlorobenzyl)-6-(4-morpholinylmethyl)-4-oxo-4H-pyrano[2,3-b]pyridine-3-carboxamide;
(68) N-(4-chlorobenzyl)-6-(4-morpholinylmethyl)-4-oxo-4H-thiochromene-3-carboxamide;
(69) N-(4-chlorobenzyl)-6-(3-hydroxypropyl)-4-oxo-4H-thiopyrano[2,3-b]pyridine-3-carboxamide;
(70) N-(4-chlorobenzyl)-6-(3-hydroxy-1-propynyl)-4-oxo-4H-thiopyrano[2,3-b]pyridine-3-carboxamide;
(71) N-(4-chlorobenzyl)-4-oxo-6-(tetrahydro-2H-pyran-4-ylmethyl)-4H-thiopyrano[2,3-b]pyridine-3-carboxamide;
(72) N-(4-chlorobenzyl)-6-(4-morpholinylmethyl)-4-oxo-4H-thiopyrano[2,3-b]pyridine-3-carboxamide;
(73) N-(4-chlorobenzyl)-6-(3-hydroxy-1-propynyl)-4-oxo-4H-1,2-benzoxazine-3-carboxamide;
(74) N-(4-chlorobenzyl)-6-(3-hydroxypropyl)-4-oxo-4H-1,2-benzoxazine-3-carboxamide;
(75) N-(4-chlorobenzyl)-6-(4-morpholinylmethyl)-4-oxo-4H-1,2-benzoxazine-3-carboxamide;
(76) N-(4-chlorobenzyl)-4-oxo-6-(tetrahydro-2H-pyran-4-ylmethyl)-4H-1,2-benzoxazine-3-carboxamide;
(77) N-(4-chlorobenzyl)-6-(3-hydroxy-1-propynyl)-4-oxo-4H-1,2-benzthiazine-3-carboxamide;
(78) N-(4-chlorobenzyl)-6-(3-hydroxypropyl)-4-oxo-4H-1,2-benzthiazine-3-carboxamide;
(79) N-(4-chlorobenzyl)-6-(4-morpholinylmethyl)-4-oxo-4H-1,2-benzthiazine-3-carboxamide;
(80) N-(4-chlorobenzyl)-4-oxo-6-(tetrahydro-2H-pyran-4-ylmethyl)-4H-1,2-benzthiazine-3-carboxamide;
(81) N-(4-chlorobenzyl)-4-hydroxy-6-(3-hydroxy-1-propynyl)-1-methyl-1H-2,1-benzothiazine-3-carboxamide 2,2-dioxide;
(82) N-(4-chlorobenzyl)-4-hydroxy-1-methyl-6-(4-morpholinylmethyl)-1H-2,1-benzothiazine-3-carboxamide 2,2-dioxide;
(83) N-(4-chlorobenzyl)-4-methyl-7-(4-morpholinylmethyl)-4H-1,4-benzothiazine-2-carboxamide 1-oxide;
(84) N-(4-chlorobenzyl)-1-methyl-6-(4-morpholinylmethyl)-1H-4,1,2-benzothiadiazine-3-carboxamide 4,4-Dioxide;
(85) N-(4-chlorobenzyl)-1-methyl-6-(4-morpholinylmethyl)-1H-thieno[2,3-e][1,3,4]thiadiazine-3-carboxamide 4,4-dioxide;
(86) N-(4-chlorobenzyl)-6-(4-morpholinylmethyl)-1H-thieno[2,3-e][1,3,4]thiadiazine-3-carboxamide 4,4-dioxide;
(87) N-(4-chlorobenzyl)-6-(3-hydroxypropyl)-1H-thieno[2,3-e][1,3,4]thiadiazine-3-carboxamide 4,4-dioxide;
(88) N-(4-chlorobenzyl)-6-(3-hydroxy-1-propynyl)-1H-thieno[2,3-e][1,3,4]thiadiazine-3-carboxamide 4,4-dioxide;
(89) N-(4-chlorobenzyl)-6-(3-hydroxypropyl)-1-methyl-1H-thieno[2,3-e][1,3,4]thiadiazine-3-carboxamide 4,4-dioxide;
(90) N-(4-chlorobenzyl)-6-(3-hydroxy-1-propynyl)-1-methyl-1H-thieno[2,3-e][1,3,4]thiadiazine-3-carboxamide 4,4-dioxide;
(91) N-(4-chlorobenzyl)-6-(3-hydroxypropyl)-1,8-dimethyl-4,7-dioxo-1,4,7,8-tetrahydro [1,8]naphthyridine-3-carboxamide;
(92) N-(4-chlorobenzyl)-6-(3-hydroxy-1-propynyl)-1,8-dimethyl-4,7-dioxo-1,4,7,8-tetrahydro[1,8]naphthyridine-3-carboxamide;
(93) N-(4-chlorobenzyl)-1,8-dimethyl-6-(4-morpholinylmethyl)-4,7-dioxo-1,4,7,8-tetrahydro[1,8]naphthyridine-3-carboxamide;
(94) N-(4-chlorobenzyl)-7-(3-hydroxy-1-propynyl)-4-oxo-4H-pyrido[2,1-c][1,2,4]triazine-3-carboxamide;
(95) N-(4-chlorobenzyl)-7-(3-hydroxypropyl)-4-oxo-4H-pyrido[2,1-c][1,2,4]triazine-3-carboxamide;
(96) N-(4-chlorobenzyl)-7-(4-morpholinylmethyl)-4-oxo-4H-pyrido[2,1-c][1,2,4]triazine-3-carboxamide;
(97) N-(4-chlorobenzyl)-4-hydroxy-2-(4-morpholinylmethyl)-1-benzothiophene-5-carboxamide;
(98) N-(4-chlorobenzyl)-4-hydroxy-2-(3-hydroxypropyl)-1-benzothiophene-5-carboxamide;
(99) N-(4-chlorobenzyl)-2-(4-morpholinylmethyl)-5-oxo-5H-[1,3]thiazolo[3,2-a]pyrimidine-6-carboxamide;
(100) N-(4-chlorobenzyl)-5-hydroxy-2-(4-morpholinylmethyl)-7-oxo-7H-[1,3,4]thiadiazolo[3,2-a]pyrimidine-6-carboxamide;
(101) N-(4-chlorobenzyl)-4-methyl-2-(4-morpholinylmethyl)-7-oxo-4,7-dihydro[1,3]thiazolo[5,4-b]pyridine-6-carboxamide;
(102) N-(4-chlorobenzyl)-6-(3-hydroxy-1-propynyl)-1-methyl-4-oxo-1,4-dihydrothieno[2,3-c]pyridazine-3-carboxamide;
(103) N-(4-chlorobenzyl)-6-(3-hydroxypropyl)-1-methyl-4-oxo-1,4-dihydrothieno[2,3-c]pyridazine-3-carboxamide;
(104) N-(4-chlorobenzyl)-1-methyl-6-(4-morpholinylmethyl)-4-oxo-1,4-dihydrothieno[2,3-c]pyridazine-3-carboxamide;
(105) N-(4-chlorobenzyl)-6-(4-morpholinylmethyl)-4-oxo-1-phenyl-1,4-dihydrothieno[2,3-c]pyridazine-3-carboxamide;
(106) N-(4-chlorobenzyl)-1-methyl-4-oxo-6-(tetrahydro-2H-pyran-4-ylmethyl)-1,4-dihydrothieno[2,3-c]pyridazine-3-carboxamide;
(107) N-(4-chlorobenzyl)-4-hydroxy-6-(3-hydroxyprop-1-ynyl)-1-methyl-1H-thieno[2,3-c][1,2]thiazine-3-carboxamide 2,2-dioxide; or
a pharmaceutically acceptable salt thereof.

12. The method according to claim 10, wherein the compound administered is:
(1) N-(4-chlorobenzyl)-7-(3-hydroxy-1-propynyl)-4-oxo-4H-pyrido[1,2-a]pyrimidine-3-carboxamide;
(2) N-(4-chlorobenzyl)-5-hydroxy-2-(4-morpholinylmethyl)-7-oxo-7H-[1,3,4]thiadiazolo[3,2-a]pyrimidine-6-carboxamide;
(3) N-(4-chlorobenzyl)-4-hydroxy-7-(3-hydroxy-1-propynyl)-2-oxo-2H-pyrido[1,2-a]pyrimidine-3-carboxamide;
(4) N-(4-chlorobenzyl)-2-(4-morpholinylmethyl)-5-oxo-5H-[1,3]thiazolo[3,2-a]pyrimidine-6-carboxamide;
(5) N-(4-chlorobenzyl)-4-hydroxy-2-(4-morpholinylmethyl)-1-benzothiophene-5-carboxamide;
(6) N-(4-chlorobenzyl)-6-(3-hydroxy-1-propynyl)-4-oxo-4H-chromene-3-carboxamide;
(7) N-(4-chlorobenzyl)-6-(3-hydroxy-1-propynyl)-4-oxo-4H-1,2-benzoxazine-3-carboxamide;
(8) N-(4-chlorobenzyl)-6-(3-hydroxy-1-propynyl)-1-methyl-4-oxo-1,4-dihydropyrido[2,3-c]pyridazine-3-carboxamide;
(9) N-(4-chlorobenzyl)-4-methyl-2-(4-morpholinylmethyl)-7-oxo-4,7-dihydro[1,3]thiazolo[5,4-b]pyridine-6-carboxamide;
(10) N-(4-chlorobenzyl)-4-hydroxy-6-(3-hydroxyprop-1-ynyl)-1-methyl-1H-thieno[2,3-c][1,2]thiazine-3-carboxamide 2,2-dioxide;
(11) N-(4-chlorobenzyl)-1-methyl-6-(4-morpholinylmethyl)-1H-4,1,2-benzothiadiazine-3-carboxamide 4,4-Dioxide;
(12) 8-chloro-N-(4-chlorobenzyl)-1-methyl-6-(4-morpholinylmethyl)-4-oxo-1,4-dihydro1,7]naphthyridine-3-carboxamide;
(13) N-(4-chlorobenzyl)-1-methyl-6-(4-morpholinylmethyl)-4-oxo-1,4-dihydro[1,7]naphthyridine-3-carboxamide;
(14) N-(4-chlorobenzyl)-1-methyl-6,8-bis(morpholin-4-ylmethyl)-4-oxo-1,4-dihydro-1,7-naphthyridine-3-carboxamide;
(15) N-(4-chlorobenzyl)-4-hydroxy-6-(3-hydroxy-1-propynyl)-1-methyl-1H-2,1-benzothiazine-3-carboxamide 2,2-dioxide;
(16) N-(4-chlorobenzyl)-6-(3-hydroxy-1-propynyl)-1-methyl-1H-thieno[2,3-e][1,3,4]thiadiazine-3-carboxamide 4,4-dioxide;
(17) N-(4-chlorobenzyl)-6-(3-hydroxy-1-propynyl)-1-methyl-4-oxo-1,4-dihydro[1,5]naphthyridine-3-carboxamide;
(18) N-(4-chlorobenzyl)-6-(4-morpholinylmethyl)-4-oxo-1-phenyl-1,4-dihydrothieno[2,3-c]pyridazine-3-carboxamide;
(19) N-(4-chlorobenzyl)-6-(4-morpholinylmethyl)-4-oxo-4H-chromene-3-carboxamide;
(20) N-(4-chlorobenzyl)-6,8-bis(4-morpholinylmethyl)-4-oxo-4H-chromene-3-carboxamide;
(21) N-(4-chlorobenzyl)-6-(((3R)-3-hydroxypyrrolidinyl)methyl)-4-oxo-4H-chromene-3-carboxamide; or a pharmaceutically acceptable salt thereof.

13. The method according to claim 10, wherein the compound administered is:
(1) N-(4-chlorobenzyl)-1-methyl-6-(4-morpholinylmethyl)-1H-4,1,2-benzothiadiazine-3-carboxamide 4,4-Dioxide;
(2) N-(4-chlorobenzyl)-4-hydroxy-6-(3-hydroxy-1-propynyl)-1-methyl-1H-2,1-benzothiazine-3-carboxamide 2,2-dioxide;
(3) N-(4-chlorobenzyl)-6-(3-hydroxy-1-propynyl)-1-methyl-1H-thieno[2,3-e][1,3,4]thiadiazine-3-carboxamide 4,4-dioxide;
(4) N-(4-chlorobenzyl)-6-(3-hydroxy-1-propynyl)-1-methyl-4-oxo-1,4-dihydro[1,5]naphthyridine-3-carboxamide; or a pharmaceutically acceptable salt thereof.

14. The method according to claim 10, wherein the compound administered is:
(1) N-(4-chlorobenzyl)-4-hydroxy-7-(3-hydroxy-1-propynyl)-2-oxo-2H-pyrido[1,2-a]pyrimidine-3-carboxamide;
(2) N-(4-chlorobenzyl)-6-(3-hydroxy-1-propynyl)-4-oxo-4H-chromene-3-carboxamide;
(3) 8-chloro-N-(4-chlorobenzyl)-1-methyl-6-(4-morpholinylmethyl)-4-oxo-1,4-dihydro1,7]naphthyridine-3-carboxamide;
(4) N-(4-chlorobenzyl)-1-methyl-6,8-bis(morpholin-4-ylmethyl)-4-oxo-1,4-dihydro-1,7-naphthyridine-3-carboxamide;
(5) N-(4-chlorobenzyl)-6-(4-morpholinylmethyl)-4-oxo-4H-chromene-3-carboxamide;
(6) N-(4-chlorobenzyl)-6,8-bis(4-morpholinylmethyl)-4-oxo-4H-chromene-3-carboxamide;
(7) N-(4-chlorobenzyl)-6-(((3R)-3-hydroxypyrrolidinyl)methyl)-4-oxo-4H-chromene-3-carboxamide; or a pharmaceutically acceptable salt thereof.

15. The method according to claim 1, wherein the compound administered has the Formula XV 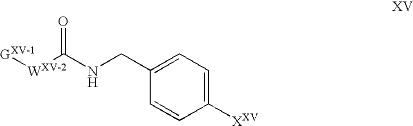
or a pharmaceutically acceptable salt thereof,
wherein,
X^{XV} is Cl, Br, F, CN or NO₂;
G^{XV} is
a) C₁₋₄alkyl which is fully saturated or partially unsaturated and is substituted by hydroxy, or
b) C₁₋₄alkyl substituted by NR^{XV-1}R^{XV-2} or 4-tetrahydropyran;
R^{XV-1} is C₂₋₇alkyl substituted by hydroxy, C₁₋₄alkoxy, or aryl;
R^{XV-2} is hydrogen or C₁₋₇alkyl; or
R^{XV-1} and R^{XV-2} together with the nitrogen to which they are attached form
a) a morpholine which may be optionally substituted by aryl^{XV} or C₁₋₇alkyl; or
b) a pyrrolidine ring substituted by hydroxy;
W^{XV} is a heterocycle of formula W1^{XV}, W2^{XV}, W3^{XV}, W4^{XV}, W5^{XV}, W6^{XV}, W7^{XV} or W8^{XV} 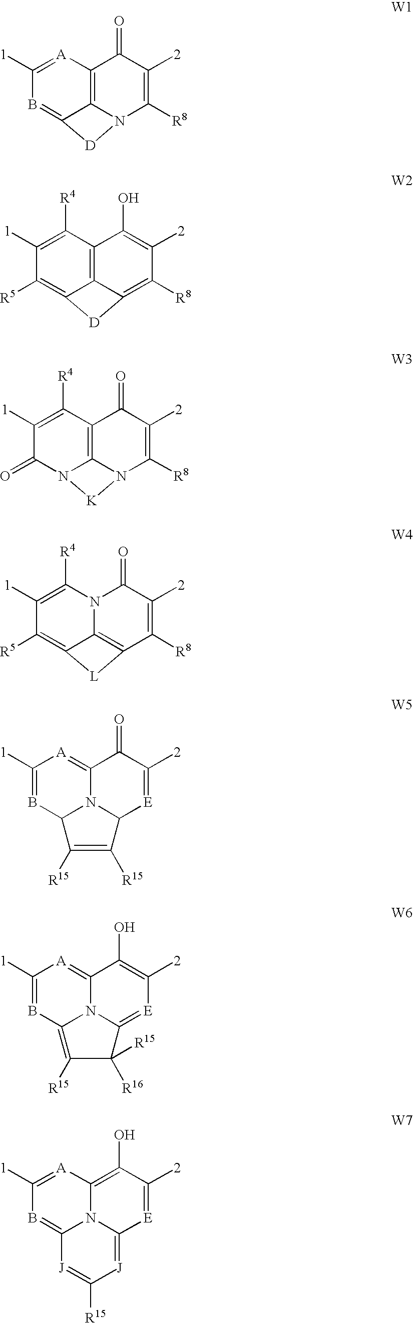 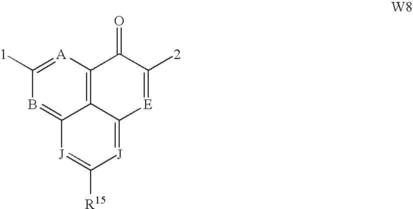
[It is intended that the R substituents and substitutents A, B, D, E, J, K and L on the W^{XV-1} to W^{XV-8} Formulae, as well as the W number under the formulas, be succeeded by the Roman Numeral XV.]
A^{XV} is CR^{XV-4} or nitrogen;
B^{XV} is CR^{XV-5} or nitrogen;
D^{XV} is
a) —(CR^{XV-13}R^{XV-14})ₐ^{XV}—, where a^{XV} is 2 or 3
b) —(CR^{XV-15}R^{XV-16})₄—,
c) —Y^{XV}—CR^{XV-13}R^{XV-14}—CR^{XV-13}R^{XV-14}—,
d) —CR^{XV-13}R^{XV-14}—Y^{XV}—CR^{XV-13}R^{XV-14}—,
e) —Y^{XV}—CR^{XV-13}R^{XV-14}—Y^{XV}—,
f) —CR^{XV-13}R^{XV-14}—CR^{XV-13}R^{XV-14}—Y^{XV}—,
g) —Y^{XV}—(CR^{XV-15}R^{XV-16})ₙ^{XV}—,
h) —Y^{XV}—CR^{XV-15}═CR^{XV-15}—,
i) —Y^{XV}—CR^{XV-15}═N—,
j) —CR^{XV-15}═CR^{XV-15}—Y^{XV}—,
k) —N═CR^{XV-15}—Y^{XV}—,
l) —(CR^{XV-15}R^{XV-16})_{b}^{XV}—N═CR^{XV-15}—, where b^{XV} is 0 or 1
m) —CR^{XV-15}═N—(CR^{XV-15}R^{XV-16})_{b}^{XV}—, where b^{XV} is 0 or 1
n) —N═N—,
o) —N═CR^{XV-15}—(CR^{XV-15}R^{XV-16})_{b}^{XV}—, where b^{XV} is 0 or 1
p) —CR^{XV-15}═CR^{XV-15}—,
q) —N═N—Y^{XV}—,
r) —Y^{XV}—N═N—,
s) —Y^{XV}—N═CR^{XV-15}—, or
t) —CR^{XV-15}R^{XV-16}—Y^{XV}—CR^{XV-15}R^{XV-16}—CR^{XV-15}R^{XV-16}—;
E^{XV} is CR^{XV-8} or nitrogen;
J^{XV} is CR^{XV-15} or nitrogen;
K^{XV} is
a) —(CR^{XV-15}R^{XV-16})ₐ^{XV}—, where a^{XV} is 2 or 3, or
b) —CR^{XV-15}═CR^{XV-15}—;
L^{XV} is
a) —(CR^{XV-15}R^{XV-16})ₐ^{XV}—, where a^{XV} is 2 or 3, or
b) —Y^{XV}—(CR^{XV-15}R^{XV-16})—(CR^{XV-15}R^{XV-16})—;
Y^{XV} is oxygen, S(O)ₘ^{XV}, or NR^{XV-7};
with the provisos that:
when W^{XV} is of formula W1^{XV}; G^{XV} is C₁₋₄alkyl which is fully saturated and is substituted by hydroxy or morpholinyl, in which morpholinyl is attached through nitrogen; A^{XV} is CR^{XV-4}; B^{XV} is CR^{XV-5}; and R^{XV-8} is hydrogen then at least one of R^{XV-13}, R^{XV-14}, or R^{XV-7} is not hydrogen or C₁₋₇alkyl;
when W^{XV} is of formula W1^{XV}, A^{XV} is CR^{XV-4}, B^{XV} is CR^{XV-5}, D^{XV} is —Y^{XV}—CR^{XV-13}R^{XV-14}—CR^{XV-13}R^{XV-14}—, and R^{XV-8} is hydrogen then Y^{XV} is not oxygen;
when W^{XV} is of formula W1^{XV}, A^{XV} is CR^{XV-4}, and B^{XV} is CR^{XV-5} then D^{XV} is not —CR^{XV-15}═CR^{XV-15}—;
R^{XV-4} is H, halogen, or C₁₋₄alkyl optionally substituted by one to three halogens;
R^{XV-5} is
a) H,
b) halo,
c) OR^{XV-12},
d) SR^{XV-12},
e) C₁₋₇alkyl which may be partially unsaturated and optionally substituted by one or more substituents selected from OR^{XV-12}, SR^{XV-12}, NR^{XV-10}R^{XV-11}, or halo,
f) C₃₋₈cycloalkyl which may be partially unsaturated and is optionally substituted by one or more substituents selected from halogen,
g) (C═O)R^{XV-9},
h) S(O)ₘ^{XV}R^{XV-9},
i) (C═O)OR^{XV-2},
j) NHSO₂R^{XV-9},
k) nitro, or
l) cyano;
R^{XV-7} is
a) H,
b) C₁₋₇alkyl which may be partially unsaturated and optionally substituted by one or more substituents selected from OR^{XV-12}, SR^{XV-12}, NR^{XV-10}R^{XV-11}, or halo,
c) C₃₋₈cycloalkyl which may be partially unsaturated and is optionally substituted by one or more substituents selected from halogen, OR^{XV-12}, SR^{XV-12}, or NR^{XV-10}R^{XV-11}, OR^{XV-12}, SR^{XV-12}, or NR^{XV-10}R^{XV-11},
d) aryl^{XV},
e) het^{XV},
f) (C═O)R^{XV-9}, or
g) S(O)ₘ^{XV}R^{XV-9};
R^{XV-8} is
a) H,
b) C₁₋₇alkyl which may be partially unsaturated and optionally substituted by one or more substituents selected from OR^{XV-12}, SR^{XV-12}, NR^{XV-10}R^{XV-11}, or halo,
c) OR^{XV-12}, or
d) SR^{XV-12};
R^{XV-9} is
a) C₁₋₇alkyl optionally substituted by OR^{XV-12} or NR^{XV-2}R^{XV-2},
b) C₃₋₈cycloalkyl optionally substituted by OR^{XV-12} or NR^{XV-2}R^{XV-2},
c) NR^{XV-10}R^{XV-11},
d) aryl^{XV}, or
e) het^{XV}, wherein said hetXV is bound through a carbon atom;
R^{XV-10} and R^{XV-11} are independently
a) H,
b) aryl^{XV},
c) C₁₋₇alkyl which may be partially unsaturated and is optionally substituted by one or more substituents selected from CONR^{XV-2}R^{XV-2}, CO₂R^{XV-2}, het^{XV}, aryl^{XV}, cyano, or halo,
d) C₂₋₇alkyl which may be partially unsaturated and is substituted by one or more substituents selected from NR^{XV-2}R^{XV-2}, OR^{XV-2}, or SR^{XV-2},
e) C₃₋₈cycloalkyl which may be partially unsaturated and is optionally substituted by one or more substituents selected from halogen, OR^{XV-10}, SR^{XV-2}, or NR^{XV-2}R^{XV-2}, or
f) R^{XV-10} and R^{XV-11} together with the nitrogen to which they are attached form a het^{XV};
R^{XV-12} is
a) H,
b) aryl^{XV},
c) het^{XV},
d) C₁₋₇alkyl optionally substituted by arylXV, or halogen,
e) C₂₋₇alkyl substituted by OR^{XV-2}, SR^{XV-2}, or NR^{XV-2}R^{XV-2}, or
f) C₃₋₈cycloalkyl which may be partially unsaturated and is optionally substituted by one or more substituents selected from halogen, OR^{XV-2}, SR^{XV-2}, or NR^{XV-2}R^{XV-2};
R^{XV-13}, R^{XV-14}, R^{XV-15}, and R^{XV-16} are independently
a) H
b) C₁₋₇alkyl which may be partially unsaturated and optionally substituted by one or more OR^{XV-12}, SR^{XV-12}, NR^{XV-10}R^{XV-11}, or halo groups,
c) C₃₋₈cycloalkyl which may be partially unsaturated and is optionally substituted by one or more substituents selected from halogen, OR^{XV-12}, SR^{XV-12}, or NR^{XV-10}R^{XV-11},
d) aryl^{XV},
e) het^{XV}, wherein said het^{XV} is bound through a carbon atom,
f) OR^{XV-12},
g) SR^{XV-12},
h) NR^{XV-10}R^{XV-11};
i) (C═O)OR^{XV-2}, or
j) R^{XV-13} and R^{XV-14} or R^{XV-15} and R^{XV-16} together with the carbon to which they are attached form (C═O);
each m^{XV} is independently 0, 1 or 2;
each n^{XV} is independently 1 or 3;
aryl^{XV} is a phenyl radical or an ortho-fused bicyclic carbocyclic radical wherein at least one ring is aromatic, and aryl^{XV} may be optionally substituted with one or more substituents selected from halo, OH, cyano, NR^{XV-2}R^{XV-2}, CO₂R^{XV-2}, CF₃, C₁₋₆alkoxy, and C₁₋₆ alkyl which maybe further substituted by one to three SR^{XV-2}, NR^{XV-2}R^{XV-2}, OR^{XV-2}, or CO₂R^{XV-2} groups;
het^{XV} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocycle group, and het^{XV} may be optionally substituted with one or more substituents selected from halo, OH, cyano, phenyl, CO₂R^{XV-2}, CF₃, C₁₋₆alkoxy, oxo, oxime, and C₁₋₆alkyl which may be further substituted by one to three SR^{XV-2}, NR^{XV-2}R^{XV-2}, OR^{XV-2}, or CO₂R^{XV-2} groups;
halo or halogen is F, Cl, Br, I;
1 represents the point of attachment between W^{XV} and GXV;
2 represents the point of attachment between W^{XV} and the carbonyl group of Formula (XV),

16. The method of claim 15, wherein the compound administered is selected from the group consisting of
(1) N-(4-chlorobenzyl)-8-(4-morpholinylmethyl)-6-oxo-6H-imidazo[4,5,1-ij]quinoline-5-carboxamide;
(2) N-(4-chlorobenzyl)-8-(3-hydroxy-1-propynyl)-6-oxo-6H-imidazo[4,5,1-ij]quinoline-5-carboxamide;
(3) N-(4-chlorobenzyl)-8-(3-hydroxypropyl)-6-oxo-6H-imidazo[4,5,1-ij]quinoline-5-carboxamide;
(4) 1-amino-N-(4-chlorobenzyl)-8-(3-hydroxy-1-propynyl)-6-oxo-1,2-dihydro-6H-pyrrolo[3,2,1-ij]quinoline-5-carboxamide;
(5) 1-amino-N-(4-chlorobenzyl)-8-(3-hydroxypropyl)-6-oxo-1,2-dihydro-6H-pyrrolo[3,2,1-ij]quinoline-5-carboxamide;
(6) 1-amino-N-(4-chlorobenzyl)-8-(4-morpholinylmethyl)-6-oxo-1,2-dihydro-6H-pyrrolo[3,2,1-ij]quinoline-5-carboxamide;
(7) N-(4-chlorobenzyl)-2-(hydroxymethyl)-8-(3-hydroxy-1-propynyl)-6-oxo-1,2-dihydro-6H-pyrrolo[3,2,1-ij]quinoline-5-carboxamide;
(8) N-(4-chlorobenzyl)-8-(3-hydroxyprop-1-ynyl)-2,2-dimethyl-6-oxo-1,2-dihydro-6H-pyrrolo[3,2,1-ij]quinoline-5-carboxamide;
(9) 3-benzyl-N-(4-chlorobenzyl)-9-(3-hydroxy-1-propynyl)-7-oxo-2,3-dihydro-7H-[1,3,4]oxadiazino[6,5,4-ij]quinoline-6-carboxamide;
(10) 3-benzyl-N-(4-chlorobenzyl)-9-(3-hydroxypropyl)-7-oxo-2,3-dihydro-7H-[1,3,4]oxadiazino[6,5,4-ij]quinoline-6-carboxamide;
(11) 3-benzyl-N-(4-chlorobenzyl)-9-(4-morpholinylmethyl)-7-oxo-2,3-dihydro-7H-[1,3,4]oxadiazino[6,5,4-ij]quinoline-6-carboxamide;
(12) 3-benzyl-N-(4-chlorobenzyl)-9-(tetrahydro-2H-pyran-4-ylmethyl)-7-oxo-2,3-dihydro-7H-[1,3,4]oxadiazino[6,5,4-ij]quinoline-6-carboxamide;
(13) N-(4-chlorobenzyl)-9-(3-hydroxyprop-1-ynyl)-3-methyl-7-oxo-2,3-dihydro-7H-[1,3,4]oxadiazino[6,5,4-ij]quinoline-6-carboxamide;
(14) 3-benzyl-N-(4-chlorobenzyl)-9-(3-hydroxy-1-propynyl)-7-oxo-2,3-dihydro-7H-[1,3,4]thiadiazino[6,5,4-ij]quinoline-6-carboxamide;
(15) 3-benzyl-N-(4-chlorobenzyl)-9-(3-hydroxypropyl)-7-oxo-2,3-dihydro-7H-[1,3,4]thiadiazino[6,5,4-ij]quinoline-6-carboxamide;
(16) 3-benzyl-N-(4-chlorobenzyl)-9-(4-morpholinylmethyl)-7-oxo-2,3-dihydro-7H-[1,3,4]thiadiazino[6,5,4-ij]quinoline-6-carboxamide;
(17) 3-benzyl-N-(4-chlorobenzyl)-9-(tetrahydro-2H-pyran-4-ylmethyl)-7-oxo-2,3-dihydro-7H-[1,3,4]thiadiazino[6,5,4-ij]quinoline-6-carboxamide;
(18) N-(4-chlorobenzyl)-3-methyl-9-(morpholin-4-ylmethyl)-7-oxo-7H-[1,4]thiazino[2,3,4-ij]quinoline-6-carboxamide;
(19) N-(4-chlorobenzyl)-3-methyl-7-oxo-9-(tetrahydro-2H-pyran-4-ylmethyl)-7H-[1,4]thiazino[2,3,4-ij]quinoline-6-carboxamide;
(20) N-(4-chlorobenzyl)-9-(3-hydroxypropyl)-3-methyl-7-oxo-7H-[1,4]thiazino[2,3,4-ij]quinoline-6-carboxamide;
(21) N-(4-chlorobenzyl)-9-(3-hydroxyprop-1-ynyl)-3-methyl-7-oxo-7H-[1,4]thiazino [2,3,4-ij]quinoline-6-carboxamide;
(22) N-(4-chlorobenzyl)-9-(morpholin-4-ylmethyl)-7-oxo-2-pyridin-3-yl-7H-[1,4]thiazino[2,3,4-ij]quinoline-6-carboxamide;
(23) N-(4-chlorobenzyl)-9-(3-hydroxy-1-propynyl)-7-oxo-3-phenyl-1H,7H-[1,3]oxazino[5,4,3-ij]quinoline-6-carboxamide;
(24) N-(4-chlorobenzyl)-9-(3-hydroxypropyl)-7-oxo-3-phenyl-1H,7H-[1,3]oxazino[5,4,3-ij]quinoline-6-carboxamide;
(25) N-(4-chlorobenzyl)-7-oxo-3-phenyl-9-(tetrahydro-2H-pyran-4-ylmethyl)-1H,7H-[1,3]oxazino [5,4,3-ij]quinoline-6-carboxamide;
(26) N-(4-chlorobenzyl)-9-(4-morpholinylmethyl)-7-oxo-3-phenyl-1H,7H-[1,3]oxazino[5,4,3-ij]quinoline-6-carboxamide;
(27) N-(4-chlorobenzyl)-9-(3-hydroxy-1-propynyl)-1-methyl-7-oxo-3-phenyl-2,3-dihydro-1H,7H-pyrido[1,2,3-de]quinoxaline-6-carboxamide;
(28) N-(4-chlorobenzyl)-9-(3-hydroxypropyl)-1-methyl-7-oxo-3-phenyl-2,3-dihydro-1H,7H-pyrido[1,2,3-de]quinoxaline-6-carboxamide;
(29) N-(4-chlorobenzyl)-9-(tetrahydro-2H-pyran-4-ylmethyl)-1-methyl-7-oxo-3-phenyl-2,3-dihydro-1H,7H-pyrido[1,2,3-de]quinoxaline-6-carboxamide;
(30) N-(4-chlorobenzyl)-9-(4-morpholinylmethyl)-1-methyl-7-oxo-3-phenyl-2,3-dihydro-1H,7H-pyrido[1,2,3-de]quinoxaline-6-carboxamide;
(31) N-(4-chlorobenzyl)-9-(3-hydroxy-1-propynyl)-2,3,7-trioxo-2,3-dihydro-1H,7H-pyrido[1,2,3-de]quinoxaline-6-carboxamide;
(32) N-(4-chlorobenzyl)-9-(3-hydroxypropyl)-2,3,7-trioxo-2,3-dihydro-1H,7H-pyrido[1,2,3-de]quinoxaline-6-carboxamide;
(33) N-(4-chlorobenzyl)-9-(4-morpholinylmethyl)-2,3,7-trioxo-2,3-dihydro-1H,7H-pyrido[1,2,3-de]quinoxaline-6-carboxamide;
(34) N-(4-chlorobenzyl)-9-(3-hydroxy-1-propynyl)-2,7-dioxo-2,3-dihydro-1H,7H-pyrido[1,2,3-de]quinoxaline-6-carboxamide;
(35) N-(4-chlorobenzyl)-9-(3-hydroxypropyl)-2,7-dioxo-2,3-dihydro-1H,7H-pyrido[1,2,3-de]quinoxaline-6-carboxamide;
(36) N-(4-chlorobenzyl)-9-(3-hydroxy-1-propynyl)-3,7-dioxo-2,3-dihydro-1H,7H-pyrido[1,2,3-de]quinoxaline-6-carboxamide;
(37) N-(4-chlorobenzyl)-9-(3-hydroxypropyl)-3,7-dioxo-2,3-dihydro-1H,7H-pyrido[1,2,3-de]quinoxaline-6-carboxamide;
(38) N-(4-chlorobenzyl)-9-(4-morpholinylmethyl)-3,7-dioxo-2,3-dihydro-1H,7H-pyrido[1,2,3-de]quinoxaline-6-carboxamide;
(39) N-(4-chlorobenzyl)-9-(4-morpholinylmethyl)-2,7-dioxo-2,3-dihydro-1H,7H-pyrido[1,2,3-de]quinoxaline-6-carboxamide;
(40) N-(4-chlorobenzyl)-9-(3-hydroxy-1-propynyl)-1-methyl-2,7-dioxo-2,3-dihydro-1H,7H-pyrido[1,2,3-de]quinoxaline-6-carboxamide;
(41) 3-benzyl-N-(4-chlorobenzyl)-9-(3-hydroxy-1-propynyl)-1-methyl-7-oxo-2,3-dihydro-1H,7H-[1,2,4]triazino[5,6,1-ij]quinoline-6-carboxamide;
(42) 3-benzyl-N-(4-chlorobenzyl)-9-(3-hydroxypropyl)-1-methyl-7-oxo-2,3-dihydro-1H,7H-[1,2,4]triazino[5,6,1-ij]quinoline-6-carboxamide;
(43) 3-benzyl-N-(4-chlorobenzyl)-9-(4-morpholinylmethyl)-1-methyl-7-oxo-2,3-dihydro-1H,7H-[1,2,4]triazino[5,6,1-ij]quinoline-6-carboxamide;
(44) 3-benzyl-N-(4-chlorobenzyl)-9-(tetrahydro-2H-pyran-4-ylmethyl)-1-methyl-7-oxo-2,3-dihydro-1H,7H-[1,2,4]triazino[5,6,1-ij]quinoline-6-carboxamide;
(45) 1-benzyl-N-(4-chlorobenzyl)-5-(3-hydroxy-1-propynyl)-2,7-dioxo-2,3-dihydro-1H,7H-pyrido[3,2,1-ij]cinnoline-8-carboxamide;
(46) 1-benzyl-N-(4-chlorobenzyl)-5-(3-hydroxypropyl)-2,7-dioxo-2,3-dihydro-1H,7H-pyrido[3,2,1-ij]cinnoline-8-carboxamide;
(47) 1-benzyl-N-(4-chlorobenzyl)-5-(4-morpholinylmethyl)-2,7-dioxo-2,3-dihydro-1H,7H-pyrido[3,2,1-ij]cinnoline-8-carboxamide;
(48) 1-benzyl-N-(4-chlorobenzyl)-5-(tetrahydro-2H-pyran-4-ylmethyl)-2,7-dioxo-2,3-dihydro-1H,7H-pyrido[3,2,1-ij]cinnoline-8-carboxamide;
(49) N-(4-Chlorobenzyl)-5-(3-hydroxyprop-1-ynyl)-1-methyl-7-oxo-2,3-dihydro-1H,7H-pyrido[3,2,1-ij]cinnoline-8-carboxamide;
(50) N-(4-chlorobenzyl)-9-(3-hydroxy-1-propynyl)-7-oxo-3-phenyl-1H,7H-[1,3]thiazino[5,4,3-ij]quinoline-6-carboxamide;
(51) N-(4-chlorobenzyl)-9-(3-hydroxypropyl)-7-oxo-3-phenyl-1H,7H-[1,3]thiazino[5,4,3-ij]quinoline-6-carboxamide;
(52) N-(4-chlorobenzyl)-9-(tetrahydro-2H-pyran-4-ylmethyl)-7-oxo-3-phenyl-1H,7H-[1,3]thiazino[5,4,3-ij]quinoline-6-carboxamide;
(53) N-(4-chlorobenzyl)-9-(4-morpholinylmethyl)-7-oxo-3-phenyl-1H,7H-[1,3]thiazino[5,4,3-ij]quinoline-6-carboxamide;
(54) N-(4-chlorobenzyl)-9-(3-hydroxypropyl)-2-methyl-7-oxo-3-phenyl-2,3-dihydro-1H,7H-pyrido[3,2,1-ij]quinazoline-6-carboxamide;
(55) N-(4-chlorobenzyl)-9-(3-hydroxy-1-propynyl)-2-methyl-7-oxo-3-phenyl-2,3-dihydro-1H,7H-pyrido[3,2,1-ij]quinazoline-6-carboxamide;
(56) N-(4-chlorobenzyl)-9-(4-morpholinylmethyl)-2-methyl-7-oxo-3-phenyl-2,3-dihydro-1H,7H-pyrido[3,2,1-ij]quinazoline-6-carboxamide;
(57) N-(4-chlorobenzyl)-9-(tetrahydro-2H-pyran-4-ylmethyl)-2-methyl-7-oxo-3-phenyl-2,3-dihydro-1H,7H-pyrido[3,2,1-ij]quinazoline-6-carboxamide;
(58) 2-benzyl-N-(4-chlorobenzyl)-9-(4-morpholinylmethyl)-7-oxo-2,3-dihydro-1H,7H-pyrido[3,2,1-ij]quinazoline-6-carboxamide;
(59) 2-benzyl-N-(4-chlorobenzyl)-9-(4-morpholinylmethyl)-3,7-dioxo-2,3-dihydro-1H,7H-pyrido[3,2,1-ij]quinazoline-6-carboxamide;
(60) N-(4-chlorobenzyl)-9-(3-hydroxy-1-propynyl)-2-(4-morpholinyl)-7-oxo-7H-[1,3,4]thiadiazino[6,5,4-ij]quinoline-6-carboxamide;
(61) N-(4-chlorobenzyl)-9-(3-hydroxypropyl)-2-(4-morpholinyl)-7-oxo-7H-[1,3,4]thiadiazino[6,5,4-ij]quinoline-6-carboxamide;
(62) N-(4-chlorobenzyl)-2-(4-morpholinyl)-9-(4-morpholinylmethyl)-7-oxo-7H-[1,3,4]thiadiazino[6,5,4-ij]quinoline-6-carboxamide;
(63) N-(4-chlorobenzyl)-2-(4-morpholinyl)-7-oxo-9-(tetrahydro-2H-pyran-4-ylmethyl)-7H-[1,3,4]thiadiazino[6,5,4-ij]quinoline-6-carboxamide;
(64) N-(4-chlorobenzyl)-9-(3-hydroxypropyl)-7-oxo-2,3-dihydro-1H,7H-pyrazino[3,2,1-ij][1,7]naphthyridine-6-carboxamide;
(65) N-(4-chlorobenzyl)-9-(4-morpholinylmethyl)-7-oxo-2,3-dihydro-1H,7H-pyrazino[3,2,1-ij][1,7]naphthyridine-6-carboxamide;
(66) N-(4-chlorobenzyl)-9-(3-hydroxy-1-propynyl)-7-oxo-2,3-dihydro-1H,7H-pyrazino[3,2,1-ij][1,7]naphthyridine-6-carboxamide;
(67) N-(4-chlorobenzyl)-9-(3-hydroxypropyl)-7-oxo-2,3-dihydro-7H-[1,4]thiazino[2,3,4-ij][1,7]naphthyridine-6-carboxamide;
(68) N-(4-chlorobenzyl)-9-(4-morpholinylmethyl)-7-oxo-2,3-dihydro-7H-[1,4]thiazino[2,3,4-ij][1,7]naphthyridine-6-carboxamide;
(69) N-(4-chlorobenzyl)-9-(3-hydroxy-1-propynyl)-7-oxo-2,3-dihydro-7H-[1,4]thiazino[2,3,4-ij][1,7]naphthyridine-6-carboxamide;
(70) N-(4-chlorobenzyl)-9-(3-hydroxy-1-propynyl)-7-oxo-2,3-dihydro-7H-[1,4]oxazino[2,3,4-ij][1,7]naphthyridine-6-carboxamide;
(71) N-(4-chlorobenzyl)-9-(4-morpholinylmethyl)-7-oxo-2,3-dihydro-7H-[1,4]oxazino[2,3,4-ij][1,7]naphthyridine-6-carboxamide;
(72) N-(4-chlorobenzyl)-9-(3-hydroxypropyl)-7-oxo-2,3-dihydro-7H-[1,4]oxazino[2,3,4-ij][1,7]naphthyridine-6-carboxamide;
(73) N-(4-chlorobenzyl)-9-(3-hydroxypropyl)-3,7-dioxo-3H,7H-pyrido[1,2,3-de]quinoxaline-6-carboxamide;
(74) N-(4-chlorobenzyl)-9-(3-hydroxy-1-propynyl)-3,7-dioxo-3H,7H-pyrido[1,2,3-de]quinoxaline-6-carboxamide;
(75) N-(4-chlorobenzyl)-9-(4-morpholinylmethyl)-3,7-dioxo-3H,7H-pyrido[1,2,3-de]quinoxaline-6-carboxamide;
(76) N-(4-chlorobenzyl)-2-[(4-chlorobenzyl)amino]-9-(4-morpholinylmethyl)-7-oxo-3H,7H-pyrido[1,2,3-de]quinoxaline-6-carboxamide;
(77) 2-(benzylamino)-N-(4-chlorobenzyl)-9-(4-morpholinylmethyl)-7-oxo-3H,7H-pyrido[1,2,3-de]quinoxaline-6-carboxamide;
(78) N-(4-chlorobenzyl)-10-(3-hydroxypropyl)-2,4,8-trioxo-1,2,3,4-tetrahydro-8H-[1,4]diazepino[3,2,1-ij]quinoline-7-carboxamide;
(79) N-(4-chlorobenzyl)-10-(3-hydroxy-1-propynyl)-2,4,8-trioxo-1,2,3,4-tetrahydro-8H-[1,4]diazepino[3,2,1-ij]quinoline-7-carboxamide;
(80) N-(4-chlorobenzyl)-10-(4-morpholinylmethyl)-2,4,8-trioxo-1,2,3,4-tetrahydro-8H-[1,4]diazepino[3,2,1-ij]quinoline-7-carboxamide;
(81) N-(4-chlorobenzyl)-10-(4-morpholinylmethyl)-2,8-dioxo-1,2,3,4-tetrahydro-8H-[1,4]diazepino[3,2,1-ij]quinoline-7-carboxamide;
(82) N-(4-chlorobenzyl)-8-(4-morpholinylmethyl)-2,6-dioxo-1,2-dihydro-6H-imidazo[4,5,1-ij]quinoline-5-carboxamide;
(83) N-(4-chlorobenzyl)-8-(3-hydroxy-1-propynyl)-2,6-dioxo-1,2-dihydro-6H-imidazo[4,5,1-ij]quinoline-5-carboxamide;
(84) N-(4-chlorobenzyl)-8-(3-hydroxypropyl)-2,6-dioxo-1,2-dihydro-6H-imidazo[4,5,1-ij]quinoline-5-carboxamide;
(85) N-(4-chlorobenzyl)-8-(4-morpholinylmethyl)-2,6-dioxo-1-[2-(1-piperidinyl)ethyl]-1,2-dihydro-6H-imidazo[4,5,1-ij]quinoline-5-carboxamide;
(86) N-(4-chlorobenzyl)-1-[2-(4-methyl-1-piperazinyl)ethyl]-8-(4-morpholinylmethyl)-2,6-dioxo-1,2-dihydro-6H-imidazo[4,5,1-ij]quinoline-5-carboxamide;
(87) N-(4-chlorobenzyl)-10-(4-morpholinylmethyl)-8-oxo-3,4-dihydro-2H,8H-[1,4]oxazepino[2,3,4-ij]quinoline-7-carboxamide;
(88) N-(4-chlorobenzyl)-3-methyl-9-(morpholin-4-ylmethyl)-7-oxo-7H-[1,4]oxazino[2,3,4-ij]quinoline-6-carboxamide;
(89) N-(4-chlorobenzyl)-3-methyl-7-oxo-9-(tetrahydro-2H-pyran-4-ylmethyl)-7H-[1,4]oxazino[2,3,4-ij]quinoline-6-carboxamide;
(90) N-(4-chlorobenzyl)-9-(3-hydroxypropyl)-3-methyl-7-oxo-7H-[1,4]oxazino[2,3,4-ij]quinoline-6-carboxamide;
(91) N-(4-chlorobenzyl)-9-(3-hydroxyprop-1-ynyl)-3-methyl-7-oxo-7H-[1,4]oxazino [2,3,4-ij]quinoline-6-carboxamide;
(92) N-(4-chlorobenzyl)-9-(morpholin-4-ylmethyl)-7-oxo-2-pyridin-3-yl-7H-[1,4]oxazino[2,3,4-ij]quinoline-6-carboxamide;
(93) 2-benzyl-N-(4-chlorobenzyl)-10-(4-morpholinylmethyl)-3,8-dioxo-1,2,3,4-tetrahydro-8H-[1,4]diazepino[6,7,1-ij]quinoline-7-carboxamide;
(94) N-(4-chlorobenzyl)-5-(3-hydroxypropyl)-4,7-dioxo-1,2-dihydro-4H,7H-imidazo[1,2,3-ij][1,8]naphthyridine-8-carboxamide;
(95) N-(4-chlorobenzyl)-5-(4-morpholinylmethyl)-4,7-dioxo-1,2-dihydro-4H,7H-imidazo[1,2,3-ij][1,8]naphthyridine-8-carboxamide;
(96) N-(4-chlorobenzyl)-5-(3-hydroxy-1-propynyl)-4,7-dioxo-1,2-dihydro-4H,7H-imidazo[1,2,3-iji][1,8]naphthyridine-8-carboxamide;
(97) N-(4-chlorobenzyl)-6-(4-morpholinylmethyl)-3-oxo-9,10-dihydro-3H,8H-pyrido [3,2,1-ij]quinoline-2-carboxamide;
(98) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide;
and pharmaceutically acceptable salts thereof.

17. The method according to claim 15, wherein the compound administered is selected from the group consisting of
(1) N-(4-chlorobenzyl)-10-(4-morpholinylmethyl)-8-oxo-3,4-dihydro-2H,8H-[1,4]oxazepino [2,3,4-ij]quinoline-7-carboxamide;
(2) N-(4-chlorobenzyl)-8-(4-morpholinylmethyl)-2,6-dioxo-1,2-dihydro-6H-imidazo[4,5,1-ij]quinoline-5-carboxamide;
(3) N-(4-chlorobenzyl)-9-(4-morpholinylmethyl)-2,7-dioxo-2,3-dihydro-1H,7H-pyrido[1,2,3-de]quinoxaline-6-carboxamide;
(4) N-(4-chlorobenzyl)-2-[(4-chlorobenzyl)amino]-9-(4-morpholinylmethyl)-7-oxo-3H,7H-pyrido[1,2,3-de]quinoxaline-6-carboxamide;
(5) N-(4-chlorobenzyl)-2-(hydroxymethyl)-8-(3-hydroxy-1-propynyl)-6-oxo-1,2-dihydro-6H-pyrrolo[3,2,1-ij]quinoline-5-carboxamide;
(6) N-(4-chlorobenzyl)-8-(3-hydroxyprop-1-ynyl)-2,2-dimethyl-6-oxo-1,2-dihydro-6H-pyrrolo[3,2,1-ij]quinoline-5-carboxamide;
(7) N-(4-chlorobenzyl)-6-(morpholin-4-ylmethyl)-3-oxo-9,10-dihydro-3H,8H-pyrido[3,2,1-ij]quinoline-2-carboxamide;
(8) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide;
(9) N-(4-chlorobenzyl)-10-(4-morpholinylmethyl)-2,8-dioxo-1,2,3,4-tetrahydro-8H-[1,4]diazepino[3,2,1-ij]quinoline-7-carboxamide;
(10) 2-(benzylamino)-N-(4-chlorobenzyl)-9-(4-morpholinylmethyl)-7-oxo-3H,7H-pyrido[1,2,3-de]quinoxaline-6-carboxamide;
(11) N-(4-chlorobenzyl)-9-(3-hydroxy-1-propynyl)-1-methyl-2,7-dioxo-2,3-dihydro-1H,7H-pyrido[1,2,3-de]quinoxaline-6-carboxamide;
(12) 2-benzyl-N-(4-chlorobenzyl)-9-(4-morpholinylmethyl)-7-oxo-2,3-dihydro-1H,7H-pyrido[3,2,1-ij]quinazoline-6-carboxamide;
(13) 2-benzyl-N-(4-chlorobenzyl)-9-(4-morpholinylmethyl)-3,7-dioxo-2,3-dihydro-1H,7H-pyrido[3,2,1-ij]quinazoline-6-carboxamide;
(14) 2-benzyl-N-(4-chlorobenzyl)-10-(4-morpholinylmethyl)-3,8-dioxo-1,2,3,4-tetrahydro-8H-[1,4]diazepino[6,7,1-ij]quinoline-7-carboxamide;
(15) N-(4-chlorobenzyl)-9-(3-hydroxyprop-1-ynyl)-3-methyl-7-oxo-2,3-dihydro-7H-[1,3,4]oxadiazino[6,5,4-ij]quinoline-6-carboxamide;
(16) N-(4-Chlorobenzyl)-5-(3-hydroxyprop-1-ynyl)-1-methyl-7-oxo-2,3-dihydro-1H,7H-pyrido[3,2,1-ij]cinnoline-8-carboxamide;
and pharmaceutically acceptable salts thereof.

18. The method according to claim 15, wherein the compound administered is selected from the group consisting of
(1) N-(4-chlorobenzyl)-10-(4-morpholinylmethyl)-8-oxo-3,4-dihydro-2H,8H-[1,4]oxazepino [2,3,4-ij]quinoline-7-carboxamide;
(2) N-(4-chlorobenzyl)-9-(4-morpholinylmethyl)-2,7-dioxo-2,3-dihydro-1H,7H-pyrido[1,2,3-de]quinoxaline-6-carboxamide;
(3) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide;
(4) N-(4-chlorobenzyl)-10-(4-morpholinylmethyl)-2,8-dioxo-1,2,3,4-tetrahydro-8H-[1,4]diazepino[3,2,1-ij]quinoline-7-carboxamide;
(5) 2-(benzylamino)-N-(4-chlorobenzyl)-9-(4-morpholinylmethyl)-7-oxo-3H,7H-pyrido[1,2,3-de]quinoxaline-6-carboxamide;
(6) N-(4-chlorobenzyl)-9-(3-hydroxy-1-propynyl)-1-methyl-2,7-dioxo-2,3-dihydro-1H,7H-pyrido[1,2,3-de]quinoxaline-6-carboxamide;
and pharmaceutically acceptable salts thereof.

19. The method according to claim 15, wherein the compound administered is selected from the group consisting of
(1) N-(4-chlorobenzyl)-9-(4-morpholinylmethyl)-2,7-dioxo-2,3-dihydro-1H,7H-pyrido[1,2,3-de]quinoxaline-6-carboxamide;
(2) N-(4-chlorobenzyl)-3-methyl-9-(4-morpholinylmethyl)-2,7-dioxo-2,3-dihydro-1H,7H-pyrido[1,2,3-de]quinoxaline-6-carboxamide hydrobromide;
and pharmaceutically acceptable salts thereof.

20. The method according to claim 1, wherein said mammal is a human.

21. The method according to claim 1, wherein said mammal is a livestock or companion animal.

22. The method according to claim 1, wherein the amount administered is from about 0.1 to about 300 mg/kg of body weight.

23. The method according to claim 1, wherein the amount administered is from about 1.0 to about 30 mg/kg of body weight.

24. The method according to claim 1, wherein the compound is administered parenterally, topically, intravaginally, orally, or rectally.
